(19) United States
(12) Patent Application Publication
(10) Pub. No.: US 2010/0209436 A1
(43) Pub. Date: Aug. 19, 2010

(54) METHOD FOR TREATING DISEASES RELATED TO MITOCHONDRIAL DYSFUNCTION

(76) Inventors: Andreas Reichert, Frankfurt am Main (DE); Stéphane Duvezin-Caubet, Pessac (FR); Johannes Wagener, Munchen (DE); Michael Zick, Germering (DE); Thomas Langer, Koln (DE); Mirko Koppen, Koln (DE); Walter Neupert, Germering (DE)

Correspondence Address: FOLEY AND LARDNER LLP;SUITE 500, 3000 K STREET NW, WASHINGTON, DC, 20007, US

(21) Appl. No.: 12/667,329

(22) PCT Filed: Jul. 2, 2008
(86) PCT No.: PCT/EP2008/005400

§ 371 Date: Dec. 30, 2009

Related U.S. Application Data
(60) Provisional application No. 60/947,933, filed on Jul. 3, 2007.

(51) Int. Cl. A61K 39/395; G01N 33/53; A61K 31/7088; A61K 38/16; C07H 21/04; C07K 16/00; C07K 14/00; A61P 43/00

(52) U.S. Cl. 424/172.1; 435/7.1; 514/44 R; 514/12; 536/23.1; 530/389.1; 530/350

(57) ABSTRACT

The present invention relates to means and methods for therapeutic intervention of mitochondrial disorders or diseases, in particular to a method for the treatment, prevention and/or amelioration of a disorder or disease correlated with mitochondrial dysfunction, a mitochondrial disorder or disease or a disorder or disease characterized by an altered OPA1 processing. Thereby, a pharmaceutically active amount of a compound capable of modulating the activity of an oligomeric complex comprising Afg3I1 and/or Afg3I2 or (a) variant(s) thereof is administered to a patient in need of medical intervention. The present invention also relates to the use of an oligomeric complex comprising Afg3I1 and/or Afg3I2 or (a) variant(s) thereof for the preparation of a pharmaceutical composition for the mentioned therapeutic intervention. The present invention further relates to a method of screening for a compound capable of modulating the activity of an oligomeric complex comprising Afg3I1 and/or Afg3I2 or (a) variant(s) thereof comprising the use of OPA1.

The present invention relates to means and methods for therapeutic intervention of mitochondrial disorders or diseases, in particular to a method for the treatment, prevention and/or amelioration of a disorder or disease correlated with mitochondrial dysfunction, a mitochondrial disorder or disease or a disorder or disease characterized by an altered OPA1 processing. Thereby, a pharmaceutically active amount of a compound capable of modulating the activity of an oligomeric complex comprising Afg3l1 and/or Afg3l2 or (a) variant(s) thereof is administered to a patient in need of medical intervention. The present invention also relates to the use of an oligomeric complex comprising Afg3l1 and/or Afg3l2 or (a) variant(s) thereof for the preparation of a pharmaceutical composition for the mentioned therapeutic intervention. The present invention further relates to a method of screening for a compound capable of modulating the activity of an oligomeric complex comprising Afg3l1 and/or Afg3l2 or (a) variant(s) thereof comprising the use of OPA1.

Mitochondria form large networks of interconnected tubules that are maintained by balanced fission and fusion events (Nunnari 1997 Mol Biol Cell 8, 1233-1242; Okamoto 2005 Annu Rev Genet. 39, 503-536). The morphology and ultrastructure of mitochondria depend on the tissue, on the physiological condition of the cell, and in particular on the functional status of mitochondria. Dynamic processes associated with mitochondria are apparently crucial for the cell, e.g. in apoptosis (Frank 2001 Dev Cell 1, 515-525; Karbowski 2002 J Cell Biol 159, 931-938; Lee 2004 Mol Biol Cell 15, 5001-5011; Jagasia 2005 Nature 433, 754-760). Likewise, formation of dendritic spines and synapses (Li 2004 Cell 119, 873-887; Verstreken 2005 Neuron 47, 365-378) and functional complementation of mitochondrial DNA (mtDNA) mutations by content mixing (Nakada 2001 Nat Med 7, 934-940; Ono 2001 Nat Genet. 28, 272-275) depend on dynamics of mitochondria. On the other hand, vast morphological alterations of mitochondria have been reported to occur in human disorders. Impairment of mitochondrial fusion or fission is causative of various neurodegenerative diseases such as Charcot-Marie-Tooth disease type 2A and 4A, and optic atrophy type 1 (Alexander 2000 Nat Genet. 26, 211-215; Delettre 2000 Nat Genet. 26, 207-210; Zuchner 2004 Nat Genet. 36, 449-451; Niemann 2005 J Cell Biol 170, 1067-1078). One key player in regulating dynamic changes of mitochondrial morphology is the protein OPA1 which is required for mitochondrial fusion (Olichon 2003J. Biol. Chem. 278, 7743-7746; Cipolat 2004 Proc Natl Acad Sci USA 101, 15927-15932). Mutations in the OPA1 gene cause autosomal dominant optic atrophy type I, a prevalent hereditary neuropathy of the optic nerve (Alexander loc cit; Delettre 2000 loc cit). Downregulation of OPA1 leads to fragmentation of mitochondria, mitochondrial dysfunction, altered maintenance of mtDNA, altered mitochondrial inner membrane morphology and increased propensity for apoptosis (Olichon 2003 loc cit; Griparic 2004 J Biol Chem 279, 18792-18798; Lee loc cit; Arnoult 2005 J Biol Chem 280, 35742-35750; Chen 2005 J Biol Chem 280, 26185-26192). Eight alternatively spliced mRNAs transcribed from the OPA1 gene were reported (Delettre 2001 Hum Genet. 109, 584-591; Olichon 2002 FEBS Lett 523, 171-176; Satoh 2003 Biochem Biophys Res Commun 300, 482-493) resulting in five apparent isoforms of the OPA1 protein (Delettre 2001 loc cit; Olichon 2003 loc cit; Duvezin-Caubet 2006 J Biol. Chem. 281(49):37972-9; Ishihara 2006 Embo J 25, 2966-2977; Olichon 2007 Cell Death Differ 14(4):682-92). There is evidence suggesting that OPA1 undergoes limited proteolysis. Dissipation of the mitochondrial membrane potential induces a fast and specific proteolytic conversion of larger OPA1 isoforms into smaller ones accompanied by a simultaneous fragmentation of mitochondria (Duvezin-Caubet loc cit; Ishihara loc cit). Proteolysis of OPA1 is observed in patients and in various model systems of human disorders associated with mitochondrial dysfunction (Duvezin-Caubet loc cit). It was further shown that mitochondrial dysfunction leads to OPA1 processing, inhibition of mitochondrial fusion, and therefore to segregation of damaged mitochondria from the network of intact mitochondria (Duvezin-Caubet loc cit). Thus, even of the fact that OPA1 processing apparently has a key role in regulating mitochondrial morphology, it is eventually not clear which proteases are indeed involved in this process. Apparently contradicting results have been reported in this respect, as PARL, a mitochondrial rhomboid protease, as well as paraplegin, a subunit of the m-AAA protease, were proposed to be involved in cleaving OPA1 (Cipolat 2006 Cell 126 163-175; Ishihara loc cit). PARL appears to be an obvious candidate for OPA1 processing as its ortholog, Pcp1, was shown to process the ortholog of OPA1, Mgm1, in baker's yeast (Herlan 2003 J Biol Chem 278, 27781-27788; McQuibban 2003 Nature 423, 537-541; Sesaki 2003 Biochem Biophys Res Commun 308, 276-283; Herlan 2004 J Cell Biol 165, 167-173). Deletion of PARL in Drosophila led to fragmentation of mitochondria (McQuibban 2006 Curr Biol 16, 982-989). Moreover, PARL is a critical regulator of OPA1-dependent cristae remodeling during apoptosis, a process that is accompanied by the accumulation of small amounts of a soluble form of OPA1 in the intermembrane space (Cipolat loc cit; Frezza 2006 Cell 126 177-189). Other observations, however, challenged the requirement of PARL for OPA1 processing. Deletion of PARL in mouse did not have an obvious effect on mitochondrial morphology (Cipolat loc cit). Further, cleavage of OPA1 has recently been linked to the hetero-oligomeric m-AAA protease (Ishihara loc cit), an ATP-dependent metalloprotease in the inner membrane of mitochondria (Atorino 2003 J Cell Biol 163, 777-787). Ishihara and colleagues (loc cit) observed impaired OPA1 processing in human cells upon downregulation of paraplegin, a subunit of the m-AAA protease, but not when PARL was downregulated: Notably, deletion of paraplegin is causative for axonal degeneration in hereditary spastic paraplegia (Casari 1998 Cell 93, 973-983) and leads to the accumulation of aberrant mitochondria in a paraplegin-deficient mouse model (Ferreirinha 2004 J Clin Invest 113, 231-242).

The technical problem underlying the present invention is the provision of suitable means and methods for therapeutic intervention against mitochondrial dysfunction and diseases or disorders related thereto. In particular, means and methods for the treatment, prevention and/or amelioration of mitochondrial dysfunction and diseases or disorders related thereto are of need.

The solution to the above technical problem is achieved by providing the embodiments characterized in the claims.

The present invention solves the above identified technical problem since, as documented herein below and in the appended examples, it was surprisingly found that OPA1 upon expression in yeast is cleaved by oligomeric m-AAA protease complexes comprising murine or human Afg3l1 and/or Afg3l2 subunits at high efficiencies.

Thus, in a first main aspect, the present invention relates to a method for the treatment, prevention and/or amelioration of
(i) a disorder or disease correlated with mitochondrial dysfunction or a mitochondrial disorder or disease; or
(ii) a disorder or disease characterized by an altered OPA1 processing, wherein said method comprises the administration to a patient in need of medical intervention a pharmaceutically active amount of a compound capable of (specifically) modulating the activity of an oligomeric complex comprising Afg3l1 and/or Afg3l2 or (a) variant(s) thereof.

From the prior art, it was known that PARL is able to cleave Mgm1, the yeast ortholog of OPA1 (McQuibban loc cit). Moreover, it has been suggested that a soluble OPA1 isoform generated by PARL cleavage in low amounts might be responsible for the anti-apoptotic effects of OPA1 (Cipolat loc cit). It was further demonstrated in the prior art that that OPA1 processing was weakly affected upon downregulation of the m-AAA protease subunit paraplegin in HeLa cells (Ishihara loc cit). In the same context, it was inter alia emphasized that the function of the ATPase family gene 3-like 2 (Afg3l2), a gene related to the paraplegin gene, remains unknown, since hints have been found that Afg3l2 is not involved in OPA1 processing (Ishihara loc cit).

In distinct differences to and against obvious conclusions to be drawn from the prior art, it was shown herein that the rhomboid-like protease PARL does not cleave OPA1 upon expression in yeast. Particularly, it was demonstrated that none of the OPA1 splice variants investigated was converted to smaller isoforms in the presence of proteolytically active PARL.

The main inventive merit of the present invention was the identification of a protease capable of efficiently cleaving, and therefore processing, OPA1, namely an oligomeric protease complex comprising Afg3l2 and/or Afg3l1.

As described herein and documented in the appended examples, OPA1 processing in yeast was reconstituted and in parallel the OPA1 processing process was analyzed in PARL and paraplegin-deficient mammalian cell lines for this purpose. The corresponding results demonstrate that PARL can functionally substitute for the yeast rhomboid Pcp1, consistent with an earlier report (McQuibban loc cit), but does not affect OPA1 processing. The corresponding results further demonstrate that mouse and mammalian m-AAA proteases mediate OPA1 cleavage when expressed in yeast. Thereby, it was surprisingly found that OPA1 processing can be effected by (homo-)oligomeric m-AAA proteases comprising Afg3l1 and/or Afg3l2 at high efficiency and to a lower extent by hetero-oligomeric, paraplegin-containing, m-AAA proteases

Without being bound by theory, this may be due to different substrate specificities of these different proteolytic complexes. Possibly, OPA1 cleavage by homooligomeric m-AAA proteases may rationalize efficient OPA1 processing particularly in paraplegin-deficient cell lines.

In addition to this, the present invention is, inter alia, based on the following particular findings:

Mass spectrometric characterization of OPA1 isoforms revealed their formation by alternative splicing and proteolytic processing in HeLa cells. Moreover, yeast was also established as a valid model system for the analysis of OPA1 processing. Using this system, it was particularly demonstrated that OPA1 is recognized and cleaved in the inner membrane by m-AAA proteases, particularly by (homo-)oligo-meric m-AAA protease complexes comprising Agf3l1/2.

As one particular aspect, it was found that certain OPA1 processing products are preferentially formed depending on the splice variant analyzed and on the subunit composition of the m-AAA protease. It was known that the expression of m-AAA protease subunits varies in different murine tissues (Koppen 2007 Mol Cell Biol. 27(2):758-67). Without being bound by theory, it is therefore conceivable on the basis of the teaching provided herein that hetero-oligomeric forms of m-AAA proteases are crucial for OPA1 processing in some tissues and not in others. Tissue-specific differences in the subunit composition of m-AAA protease isozymes as well as in the expression of OPA1 isoforms could explain why deficiencies in paraplegin in mouse and human do result in cell type specific mitochondrial defects.

In view of the different efficiencies of OPA1 processing by m-AAA proteases composed of different subunits, it is, without being bound by theory, conceivable that variation in the assembly of m-AAA proteases allows adjusting OPA1 processing and thereby mitochondrial dynamics to different needs in different tissues. Moreover, the m-AAA protease may play additional roles during OPA1 cleavage which cannot be carried out by, for example, PARL. The m-AAA protease is known to mediate the ATP-dependent dislocation of proteins from the membrane to allow their complete proteolysis in a hydrophilic environment (Leonhard 2000 Mol Cell 5, 629-638). Interestingly, the ATP-dependent membrane dislocation of cytochrome c peroxidase by the m-AAA protease in yeast was recently found to facilitate maturation by the rhomboid protease Pcp1 (Tatsuta 2007 Embo J 26, 325-335). However, as OPA1 is not recognized and cleaved by PARL in yeast, the results provided herein do not favor such a functional interplay between both rhomboid and AAA proteases during OPA1 processing in mammalian mitochondria. In contrast to the situation in yeast, where short forms of the OPA1 ortholog, Mgm1, are formed during biogenesis (Herlan loc cit), in mammalian cells preexisting large forms of OPA1 can be rapidly converted to small forms, e.g. upon dissipation of the membrane potential across the inner membrane (Duvezin-Caubet loc cit). The latter appears to be the consequence of a signaling pathway induced by mitochondrial dysfunction. Possibly, this mechanism has evolved only in higher organisms and depends on the m-AAA rather than the rhomboid protease as it allows for fast adaptation of mitochondrial morphology.

In view of the findings made in context of this invention, an oligomeric complex comprising Afg3l1 and/or Afg3l2 or a variant(s) thereof, as well as compound capable of (specifically) modulating the activity thereof, are particularly promising candidates for therapeutic intervention with respect to disorders or diseases correlated with mitochondrial dysfunction or mitochondrial disease and disorders characterized by an altered OPA1 processing, respectively.

Furthermore, based on the findings provided herein, it is also envisaged to take advantage of a compound capable to modulate fusion or fission of mitochondria within the mitochondrial network. For example, such a compound may be one that alters a given status quo of OPA1 processing, like, e.g. an agonist or antagonist as definded herein, or OPA1 itself or a variant or derivative thereof or (an) OPA1 isoform(s) or (a) variant(s) or (a) derivative(s) thereof.

In a second main aspect, the present invention relates to the use of a compound capable of modulating the activity of an oligomeric complex comprising Afg3l1 and/or Afg 3l2 or (a) variant(s) thereof for the treatment, prevention and/or amelioration of
(i) a disorder or disease correlated with mitochondrial dysfunction or a mitochondrial disorder or disease; or
(ii) a disorder or disease characterized by an altered OPA1 processing.

Particular disorders or diseases to be therapeutically intervened, e.g. to be treated, ameliorated and/or prevented in context of the present invention may, as non-limiting examples, be neurological disorders (e.g. Alzheimer's disease, bipolar disorders, stroke, Charcot-Marie-Tooth disease, ALS or Parkinson's disease), myopathies (e.g. general myopathy, Ataxia, infantile myopathy, atrophies, ocular myopathy, motor neuron disorders, general encephalomyopathy, Leigh-Syndrom, MELAS (myopathy encephalopathy lactic acidosis and stroke-like episodes), MERRF (myoclonic epilepsy with ragged-red fiber disease) or optic atrophy type 1), metabolic disorders (e.g. diabetes or obesity), infection disorders (e.g. bacterial, fungal or viral infections), neoplastic disorders or cancers, ischemias, oxidative damages, and the like.

Further disorders or diseases correlated with mitochiondrial dysfunction or mitochondrial disorders or diseases to be treated (in vivo or in vitro) by the means and methods provided herein are given herein below.

Out of these diseases, premature ageing, cardiomyopathy, a respiratory chain disorder, mtDNA depletion syndrome, myoclonus epilepsy, ragged-red fibers syndrome (MERRF), myopathy encephalopathy lactic acidosis, stroke-like episodes (MELAS) and optic atrophy are particularly intended to be treated ameliorated and/or prevented in context of the present invention.

In addition or in iteration to the above, further clinical and/or pathological situations correlated with mitochondrial dysfunction or characterized by an altered OPA1 processing and hence, intended to be therapeutically intervened in context of this invention, are given in the table below.

Ageing; in particular pathological and/or pre-mature aging
Alzheimer's disease
Amyotrophic lateral sclerosis (ALS)
Apoptosis
Ataxia
Autism
Barth syndrome, (familial)
Bipolar disorder
Cancer (e.g. renal cell and colorectal carcinoma, early liver, protasta,
breast, bladder, primary lung, head and neck tumours, astrocytomas,
adenocarcinomas in Barrett's esophagus)
Cardiomyopathy
Charcot-Marie-Tooth disease type 2a
Charcot-Marie-Tooth disease type 4a
Congenital lactic acidosis
Crohn disease
Deafness
Diabetes
Diabetic sensory neuropathy
Encephalomyopathy
Endotoxemia
External ophthalmoplegia (e.g. PEO)
Friedreich's ataxia
Hepatopathy (e.g. defects in SCO1)
Hepato-cerebral form of mtDNA depletion syndrome
Hereditary sensory neuropathy
Hereditary spastic paraplegia
Infantile encephalopathy
Infantile myopathy
Infectious diseases
Inflammatory diseases
Ischemia-reperfusion injury/Hypoxic damage/Oxidative damage
Kearns-Sayre syndrome
Lactic acidosis
Leber's hereditary opticus neuropathy (LHON)
Leigh's syndrome
Leukodystrophy
Mohr-Tranebjaerg-syndrome
Metabolic disorders (e.g. defective glucose and fatty acid metabolism)
Mitochondrial neurogastrointestinal-encephalomyopathy
Motor neuron disorders
mtDNA depletion syndrome
Myoclonus epilepsy and ragged-red fibers syndrome (MERRF)
Myopathy
Myopathy encephalopathy lactic acidosis and stroke-like episodes
(MELAS)
Myositis
Neurodegenerative disorders (e.g. autosomal dominant optic atrophy,
Charcot-Marie-tooth disease, Wolf-Hirschhorn syndrome, ALS)
Non-alcoholic fatty liver disease
Obesity
Ocular myopathy
Optic atrophy type 1
Paraganglioma (e.g. defects in complex II/SDH)
Parkinson's disease
Pearson's syndrome
respiratory chain disorder
Rhabdomyolysis
Schizophrenia
Sideroblastic anemia
Stroke
Tubulopathy (e.g. defects in BCS1L)
Viral and bacterial infections
Wolfram syndrome

However, the disorders or diseases to be medically intervened in context of this invention are not strictly construed to the disorders described above.

It is of note that the present invention is particularly useful in the treatment, prevention and/or amelioration of a disease or disorder described herein before any clinical and/or pathological symptoms are diagonosed or determined or can be diagnosed or determined by the attending physician. Thereby, prior to the herein disclosed medical interventions, particular advantage can also be taken of the means and methods disclosed in PCT/EP2007/004466 (EP-attorney docketing no.: M1590 PCT S3; claiming priority to U.S. 60/801,484) for determining the susceptibility for, predisposition for or the presence of a corresponding disorder or disease. Therefore, the present invention is particularly useful in early treatment and/or amelioration and hence, prevention of the diseases or disorders described herein.

In a third main aspect, the present invention relates to a method of screening for a compound capable of modulating the activity of an oligomeric complex comprising Afg3l1 and/or Afg3l2 or (a) variant(s) thereof comprising the steps of
(a) contacting OPA1 with said oligomeric complex comprising Afg3l1 and/or Afg 3l2 or (a) variant(s) thereof in the presence of said compound to be screened for under conditions allowing OPA1 processing to occur (herein referred to as “test sample”); and
(b) evaluation whether OPA1 processing is altered compared to a control, wherein OPA1 and said oligomeric complex comprising Afg 3l1 and/or Afg3l2 or (a) variant(s) thereof are contacted in the absence of said compound to be screened for under conditions allowing OPA1 processing to occur (herein referred to as “control sample”).

The herein disclosed method of screening may further comprise the step of determining the extent of OPA1 processing in the test sample and in the control sample and/or the step of comparing the corresponding results from the test sample with those of the control sample. Thereby, if the extent of OPA1 processing in the test sample differs from that of the control sample, the compound to be screened for is considered to be a modulator of an “oligomeric complex comprising Afg3l1 and/or Afg3l2 or (a) variant(s) thereof”, i.e. a “compound capable of modulating the activity of an oligomeric complex comprising Afg3l1 and/or Afg3l2 or (a) variant(s) thereof” in accordance with the present invention.

If the extend of OPA1 processing in the test sample exceeds that of the control sample, the compound screened is considered to be an “agonist” of said oligomeric complex in accordance with the present invention. If the extend of OPA1 processing in the test sample falls short of that of the control sample, the compound screened is considered to be an “antagonist” of said oligomeric complex in accordance with the present invention.

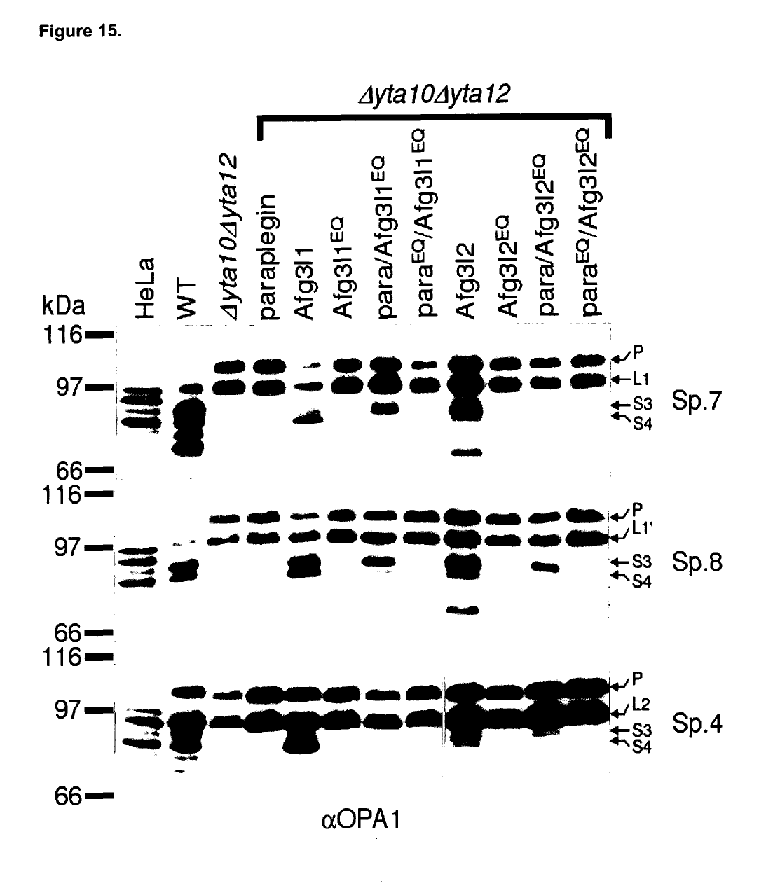

For example, the control sample takes advantage of cells where OPA1 and (a) subunit(s) of the oligomeric complex is present (e.g. expressed), like those referred to in FIG. 15 and the corresponding Examples.

The term “conditions allowing OPA1 processing to occur” means that OPA1, i.e. one or more of its spliceforms, can be proteolytically cleaved to form one or more of the OPA1 isoforms, whenever an agent/compound capable to cleave OPA1, i.e. capable to trigger OPA1 processing, is present. In other words, said “conditions” are such that said agent/compound capable to cleave OPA1 is active.

Examples of systems exhibiting the above-mentioned “conditions” are provided herein and in the appended examples, e.g. in form of the cells defined and described herein. Particularly, these cells are the HeLa cells and the yeast cells provided herein and referred to in the appended examples, particularly the HeLa and yeast cells as referred to in FIG. 15 and the corresponding example.

In view of the above, it is evident for the skilled person that in context of the method of screening provided herein OPA1 and/or the herein defined oligomeric complex or (a) subunit(s) thereof is, for example, intended to be expressed in cells like the ones provided and described herein, i.e. in cells providing the above-mentioned “conditions”.

Subsequently, these cells may then be contacted with the compound to be screened in a manner that the oligomeric complex (contacted with OPA1) can get into contact with the compound to be screened. The skilled person is aware how this can be achieved. For example, the compound to be screened can be taken up into the cells expressing OPA1 and/or the oligomeric complex or (a) subunit(s) thereof (e.g. by corresponding carriers or shuttle systems or by endocytosis; or due to the membrane permeability of the compound to be screened).

Moreover, the compound to be screened can be driven into the cells by corresponding known methods (e.g. intracytoplasmic injection or electroporation). Moreover, cell free (expression) systems or in vitro (expression/translation) systems can be employed in context of the method of screening provided herein (e.g. cell free (expression) systems generated from the cells provided herein and referred to in the appended examples) and the compound to be screened can be added to these cell free (expression) systems.

In general, the term “oligomeric complex comprising Afg3l1 and/or Afg3l2 or (a) variant(s) thereof” as described and defined in context of this invention (also referred to herein as “oligomeric complex”) refers to a certain kind of protease, particularly a certain kind of m-AAA protease (matrix-ATPase family associated with various cellular activities; Marchler-Bauer 2007 Nucleic Acids Res. 35 D237-40; http://www.ncbi.nlm.nih.gov/Structure/cdd/cddsrv.cgi?uid=63893; pfam00004). The meaning of the term “m-AAA protease(s)” is well known in the art (see above). It is further known in the art that m-AAA proteases are an assembly of several subunits, i.e. proteinaceous subunits. There is particular evidence that m-AAA proteases are an assembly of 6 subunits building a hexamer. Two of these hexamers are discussed to be further aggregated to a superior complex.

In accordance with this, the term “oligomeric” as used herein means comprising or assembled by more than one subunits. The number of the subunits comprised in an “oligomeric complex” as defined herein may be at least 2, at least 3, at least 4, at least 5, at least 10, at least 12, at least 18 or at least 24. However, a preferred “oligomeric complex” in accordance with the present invention comprises 6 subunits (including for example Afg3l1 one subunit and Afg3l1, Afg3l2, or paraplegin as another subunit). However, as mentioned above, also an “oligomeric complex” comprising another number of subunits is generally envisaged to be employed in context of the present invention. In a non-limiting example, an “oligomeric complex” may comprise at least 3 subunits, for example, paraplegin and at least two other subunits being Afg3l1 and/or Afg3l2.

It is of note that at least one subunit comprised in the herein defined oligomeric complex must be proteolytically active regardless whether the remaining subunits are. Otherwise said oligomeric complex would not be proteolytically active. Irrespective whether active or not, all subunits, however, must be assembly competent with respect to said oligomeric complex.

According to the present invention, it is the main feature of the “oligomeric complex” to comprise at least one subunit being Afg3l1 or a variant thereof or Afg3l2 or a variant thereof. Further subunits making said “complex” being an “oligomeric complex” in accordance with this invention may be any kind of subunit of an m-AAA protease. As mentioned, non-limiting examples of subunits of an m-AAA protease are Afg3l1 or variants thereof, Afg3l2 or variants thereof and paraplegin or variants thereof.

From the above, it is evident that the herein described “oligomeric complex” can be a homo-oligomeric complex or a hetero-oligomeric complex. It is preferred in context of the present invention that the herein described “oligomeric complex” is a homo-oligomeric complex. Such a homo-oligomeric comprises either Afg3l2 or Afg3l1 subunits. However, also a hetero-oligomeric complex is envisaged to be employed in context of the present invention. Among the possible hetero-oligomeric complexes envisaged to be employed in context of the present invention those are preferred, which comprise Afg3l2 and Afg3l1 subunits. Less preferred are complexes comprising Afg3l2 or Afg3l1 and a further subunit, like, for example paraplegin.

The meaning of the terms “Afg3l1” and “Afg3l2” and “paraplegin” is well known in the art and is, if not explicitly prescribed differentially, used accordingly in context of the present invention. “Afg3l1” and “Afg3l2” are known to be abbreviations of ATPase family gene 3-like 1 and ATPase family gene 3-like 2 and are used accordingly. In context of this invention, these terms are likewise used to refer to the corresponding nucleotide sequences (e.g. the genes) as well as to the corresponding polypeptides (e.g. the polypeptides encoded by said genes).

It is known in the art that Afg3l1 originates from mouse (examples of database entries: NM_{—}054070 for the nucleotide sequence and NP_{—}473-411 for the amino acid sequence), that paraplegin originates form human (database entry: NM_{—}003119 (isoform 1) and NM_{—}199367 (isoform 2) for the nucleotide sequences and NP_{—}003110 (isoform 1) and NP_{—}955399 (isoform 2) for the amino acid sequences) and that Afg3l2 denotes two homologues from human (examples of database entries: NM_{—}006796 for the nucleotide sequence and NP_{—}006787 for the amino acid sequence) and mouse (examples of database entries: NM_{—}027130 for the nucleotide sequence and NP_{—}081406 for the amino acid sequence).

In addition to the above proteins it is generally intended that the herein described “oligomeric complex” may comprise other m-AAA subunits, for example m-AAA subunits originating from organisms different from mouse or human. Such different organisms are, for example, other mammals, like, for example, rat, rabbit, goat, sheep, pig, monkey etc.

However, the herein described “oligomeric complex” comprises m-AAA subunits originating preferably form mouse, more preferably from human. The terms “m-AAA subunit” as well as “Afg3l1”, “Afg3l2” and “paraplegin” are used correspondingly in context of this invention.

It is generally intended herein that the meaning of the terms “Afg3l1”, “Afg 3l2” and “paraplegin” as used herein not only encompasses Afg3l1, Afg3l2 and paraplegin itself, but also variants thereof, for example variants thereof as described and defined herein. Thereby, it is of note, that these “variants” may differ from Afg3l1, Afg3l2 and paraplegin itself only to such an extent that the herein defined “oligomeric complex” is still active when comprising at least one of said variants (for example beneath paraplegin, Afg3l1 or Afg3l2 itself as the other subunit).

In a specific embodiment of this invention, the oligomeric complex as defined and described herein comprises a polypeptide selected from the group consisting of:
(a) a polypeptide comprising an amino acid sequence as depicted in SEQ ID NO 38, 40 or 42;
(b) a polypeptide comprising an amino acid sequence encoded by a nucleic acid molecule as depicted in SEQ ID NO 37, 39 or 41;
(c) a polypeptide comprising an amino acid sequence encoded by a nucleic acid molecule encoding an amino acid sequence as depicted in SEQ ID NO 38, 40 or 42;
(d) a polypeptide comprising an amino acid sequence being homologous to the polypeptide of any one of (a) to (c);
(e) a polypeptide comprising an amino acid sequence encoded by a nucleic acid molecule being homologous to the nucleic acid molecule as defined in any one of (b) to (c);
(f) a polypeptide comprising an amino acid sequence encoded by a nucleic acid molecule hybridizing (under stringent conditions) to the complement stand of a nucleic acid molecule as defined in any one of (b) to (c); and
(g) fragment of a polypeptide of any one of (b) to (f).

The polypeptides as defined in (d) to (g) and the nucleic acid molecule as defined in (c) to (g) are, for example, “variants” in accordance with the present invention.

“Homologous” or “homology” as used in context of this invention, for example, means at least 50%, at least 60%, at least 70%, at least 80%, at least 85%, at least 90%, at least 95%, at least 98% or at least 99% identical on the level of the amino acid or nucleic acid sequence. Thereby, the higher values of percentage are preferred.

It is of note that the meaning of the terms “nucleic acid molecule”, “nucleic acid sequence” or “nucleotide sequence”, and the like, as used herein are well known in the art and, for example, comprise DNA (e.g. cDNA or gDNA) and RNA (e.g. mRNA or siRNA).

The term “variant(s)” of the subunits comprised in the “oligomeric complex” is also intended to encompass “(a) fragment(s)” of said subunits (or of the mentioned variants thereof). Thereby, the term “fragment(s)” means amino acid stretches of at least 50, at least 100, at least 150, at least 200, at least 300, at least 500 or at least 700 amino acids of the “subunits” defined herein, or nucleotide stretches of at least 150, at least 300, at least 450, at least 600, at least 900, at least 1500 or at least 2100 nucleotides of the corresponding nucleic acid sequences defined herein.

In context of the present invention the meaning of the mentioned term “variant(s)” also encompasses conservative amino acid exchanges and further known modifications.

In the context of the present invention the term “hybridizing” means that hybridization can occur between one nucleotide sequence and another (complementary) nucleotide sequence. Thereby, the term “hybridization” means hybridization under conventional hybridization conditions, preferably under stringent hybridization conditions. Such conditions are, for instance, described in Sambrook and Russell (2001), Molecular Cloning: A Laboratory Manual, CSH Press, Cold Spring Harbor, N.Y., USA. In an especially preferred embodiment, the term “hybridization” means that hybridization occurs under the following conditions:

| | |
|---|---|
| Hybridization buffer: | 2 × SSC; 10 × Denhardt solution |
| | (Fikoll 400 + PEG + BSA; |
| | ratio 1:1:1); 0.1% SDS; 5 mM EDTA; |
| | 50 mM Na₂HPO₄; |
| | 250 μg/ml of herring sperm DNA; |
| | 50 μg/ml of tRNA; |
| | or |
| | 0.25 M of sodium phosphate buffer, |
| | pH 7.2; |
| | 1 mM EDTA |
| | 7% SDS |
| Hybridization temperature T | =60° C. |
| Washing buffer: | 2 × SSC; 0.1% SDS |
| Washing temperature T | =60° C. |

As mentioned, it is particularly intended that a “variant” as defined herein still maintains the function(s) of the herein defined oligomeric complex comprising said “variant” or still has the function of the polypeptide (or the corresponding encoding nucleic acid molecule) from which it derives.

An example of a function of said oligomeric complex is the capability to cleave OPA1 proteolytically. Further examples of such functions are given herein-below.

An example of a function of said polypeptide is the capability to assemble to an m-AAA protease complex, e.g. to an oligomeric complex as defined herein.

The oligomeric complex as described and defined herein is intended to have protease activity, particularly m-AAA protease activity. In accordance with the findings provided herein and in the appended examples, the preferred activity/function of the herein defined oligomeric complex is the proteolytic cleavage of OPA1. This proteolytic cleavage particularly leads to OPA1 processing.

The meanings of terms like “OPA1”, “OPA1 processing”, “proteolytic cleavage of OPA1”, “large/small OPA1 isoforms”, and the like, are known in the art (Ishihara loc cit; Duvezin-Caubet loc cit) and can also be deduced from PCT/EP2007/004466 (EP-attorney docketing no.: M1590 PCT S3; claiming priority to U.S. 60/801,484). Moreover, the corresponding definitions given herein-below, apply here mutatis mutandis.

As mentioned above, a “compound” to be employed, i.e. to be administered, in context of this invention can be any compound “capable of (specifically) modulating the activity of an oligomeric complex comprising Afg3l1 and/or Afg3l2 or (a) variant(s) thereof”.

In one specific embodiment, such a “compound” is intended to be a compound screened for by the corresponding method of screening of this invention.

Generally, it is intended herein that a “compound capable of modulating the activity of an oligomeric complex comprising Afg3l1 and/or Afg3l2 or (a) variant(s) thereof” as employed herein is or comprises an agonist or antagonist of the activity of an oligomeric complex comprising Afg3l1 and/or Afg3l2 or (a) variant(s) thereof.

The definitions of the term “activity” given herein-above apply here, mutatis mutandis.

In a specific embodiment of this invention an “antagonist” is a molecule compound selected from the group consisting of:
(a) a binding molecule that (specifically) binds to/interacts with the oligomeric complex comprising Afg3l1 and/or Afg3l2 or (a) variant(s) thereof as defined herein or (specifically) binds to/interacts with a nucleic acid molecule encoding ((a) subunit(s) of) the oligomeric complex comprising Afg3l1 and/or Afg3l2 or (a) variant(s) thereof as defined herein;
(b) a nucleic acid molecule capable of specifically introducing an insertion of a heterologous sequence or a mutation into a nucleic acid molecule encoding ((a) subunit(s) of) the oligomeric complex comprising Afg3l1 and/or Afg3l2 or (a) variant(s) thereof as defined herein via in vivo mutagenesis;
(c) a nucleic acid molecule capable of specifically reducing the expression of mRNA encoding ((a) subunit(s) of) the oligomeric complex comprising Afg3l1 and/or Afg3l2 or (a) variant(s) thereof as defined herein by cosuppression; and
(d) a low molecular weight compound or a small molecule, for example being capable of inhibiting the activity of the oligomeric complex comprising Afg3l1 and/or Afg3l2 or (a) variant(s) thereof as defined herein.

Non-limiting examples of a binding molecule as employed in context of this invention are is selected form the group consisting of antibodies, affybodies, trinectins, anticalins, aptamers, PNA, DNA or RNA, and the like.

Based on prior art literature, the person skilled in the art is familiar with obtaining specific binding molecules that may be useful in the context of the present invention. These molecules are directed and bind/interact specifically to or specifically label the oligomeric complex as defined herein or nucleotide sequences encoding (a) subunit(s) thereof. Non-limiting examples of suitable binding molecules may be selected from aptamers (Gold, Ann. Rev. Biochem. 64 (1995), 763-797)), aptazymes, RNAi, shRNA, RNAzymes, ribozymes (see e.g., EP-B1 0 291 533, EP-A10 321 201, EP-B1 0 360 257), antisense DNA, antisense oligonucleotides, antisense RNA, siRNA, antibodies (Harlow and Lane “Antibodies, A Laboratory Manual”, CSH Press, Cold Spring Harbor, 1988), affibodies (Hansson, Immunotechnology 4 (1999), 237-252; Henning, Hum Gene Ther. 13 (2000), 1427-1439), lectins, trinectins (Phylos Inc., Lexington, Mass., USA; Xu, Chem. Biol. 9 (2002), 933), anticalins (EPB1 1 017 814), and the like.

For example, such binding molecules may, inter alia, be selected from the group consisting of:
(a) an antibody that specifically binds to the polypeptide or the nucleic acid molecule as defined herein-above or to ((a) subunit(s) of) the oligomeric complex comprising Afg3l1 and/or Afg3l2 or (a) variant(s) thereof as defined herein;
(b) an antisense nucleotide sequence that specifically hybridizes to the nucleic acid molecule as defined herein-above;
(c) a siRNA that specifically interacts with the nucleic acid molecule as defined herein-above;
(d) an aptamer that specifically binds to the polypeptide or the nucleic acid molecule as defined herein-above or to ((a) subunit(s) of) the oligomeric complex comprising Afg3l1 and/or Afg3l2 or (a) variant(s) thereof as defined herein; and
(e) a ribozyme that specifically interacts with the nucleic acid molecule as defined herein-above.

A binding molecule (for example an antibody) to be employed in context of this invention may, for example, (specifically) bind to a particular epitope of the herein defined (subunit(s) of) the oligomeric complex comprising Afg3l1 and/or Afg3l2 or (a) variant(s) thereof. Preferably, this particular epitope is essential for the activity of said complex, like, for example, an epitope comprising the active center of said complex. Moreover, such an epitope may, for example, comprise the consensus amino acid sequence of the metal binding site. This consensus amino acid sequence may, for example, be HEXXH, wherein X is any amino acid.

Further examples of epitopes to which the binding molecule to be employed in context of this invention is intended to particularly bind to are epitopes comprising the following amino acid stretches or variants thereof:

1. An amino acid stretch as disclosed in Atorino (loc cit) of the AFG3L2 gene product (amino acids 413-828 or nucleotides 413-828 of the corresponding gene).
2. The carboxyterminal-peptide of the subunit of the oligomeric complex as defined herein (for example the 15 ⁺/₋1-3 C-terminal amino acids).
3. Amino acids 121 to 139 of murine paraplegin (C-PEDDEEEKRRKEREDQMYR), amino acids 90 to 103 of Afg3l2 (C-KEAVGEKKEPQPSG) and amino acids 104 to 118 (C-NAGPGGDGGNRGGKG) or amino acids 771 to 785 (C-WNKGREEGGTERGLQ) of Afg3l1.

In this context, it is to be understood that the person skilled in the art is, based on the teaching provided herein, readily in a position to deduce (further) amino acid stretches/peptides being specific for (a particular subunit of) the oligomeric complex defined herein and therefore, representing an “epitope” as employed herein.

The antibody useful as a binding molecule in context of the present invention (commonly known as therapeutic antibody) can be, for example, polyclonal or monoclonal. The term “antibody” also comprises derivatives or fragments thereof which still retain the binding specificity. Techniques for the production of antibodies are well known in the art and described, e.g. in Harlow and Lane “Antibodies, A Laboratory Manual”, CSH Press, Cold Spring Harbor, 1988. These antibodies can be used as particular binding molecules defined herein. Surface plasmon resonance as employed in the BIAcore system can be used to increase the efficiency of phage antibodies which bind to an epitope of the polypeptide/complex employed in this invention (Schier, Human Antibodies Hybridomas 7 (1996), 97-105; Malmborg, J. Immunol. Methods 183 (1995), 7-13). Accordingly, also phage antibodies can be used in context of this invention.

The present invention furthermore includes the use of chimeric, single chain and humanized antibodies, as well as antibody fragments, like, inter alia, Fab fragments. Antibody fragments or derivatives further comprise F(ab′)2, Fv or scFv fragments; see, for example, Harlow and Lane, loc. cit. Various procedures are known in the art and may be used for the production of such antibodies and/or fragments. Thus, the (antibody) derivatives can be produced by peptidomimetics. Further, techniques described for the production of single chain antibodies (see, inter alia, U.S. Pat. No. 4,946,778) can be adapted to produce single chain antibodies to polypeptide(s) as defined in context of this invention. Also, transgenic animals may be used to express humanized antibodies against the polypeptides/subunits/complexes as described herein. Most preferably, the antibody to be employed in context of this invention is a monoclonal antibody. For the preparation of monoclonal antibodies, any technique which provides antibodies produced by continuous cell line cultures can be used. Examples for such techniques include the hybridoma technique (Köhler and Milstein Nature 256 (1975), 495-497), the trioma technique, the human B-cell hybridoma technique (Kozbor, Immunology Today 4 (1983), 72) and the EBV-hybridoma technique to produce human monoclonal antibodies (Cole, Monoclonal Antibodies and Cancer Therapy, Alan R. Liss, Inc. (1985), 77-96). Techniques describing the production of single chain antibodies (e.g., U.S. Pat. No. 4,946,778) can be adapted to produce single chain antibodies to OPA1 or OPA1 isoforms. Accordingly, in context of the present invention, the term “antibody molecule” relates to full immunoglobulin molecules as well as to parts of such immunoglobulin molecules. Furthermore, the term relates, as discussed above, to modified and/or altered antibody molecules, like chimeric and humanized antibodies. The term also relates to monoclonal or polyclonal antibodies as well as to recombinantly or synthetically generated/synthesized antibodies. The term also relates to intact antibodies as well as to antibody fragments thereof, like, separated light and heavy chains, Fab, Fab/c, Fv, Fab′, F(ab′)2. The term “antibody molecule” also comprises bifunctional antibodies, trifunctional antibodies and antibody constructs, like single chain Fvs (scFv) or antibody-fusion proteins.

Non-limiting examples of a low molecular weight compound or a small molecule to be employed as “antagonists” herein are any protease inhibitors or metal chelators (like, for example, EDTA) capable of inhibiting, preferably specifically inhibiting, the activity of the oligomeric complex described herein.

Particularly, metalloprotease inhibitors (like ortho-phenantrolin, DCI, and the like) are intended to be employed as low molecular weight compound or a small molecule in context of the present invention.

A further low molecular weight compound or a small molecule as employed in context of this invention may, for example, be a nucleotide analogon, like, for example, ATPγS, and the like.

As mentioned, in one particular embodiment, the “antagonist” to be employed is a nucleic acid molecule that leads to a reduction or depletion of the activity of the oligomeric complex defined herein via in vivo mutagenesis. Thereby, without being bound by theory, an insertion of a heterologous sequence or a mutation into a nucleotide sequence encoding a subunit of said complex, leads to a reduction of the amount of said subunit and hence, to a reduced expression of the intact complex. Generally, methods of “in vivo mutagenesis” (also known as “chimeroplasty”) are known in the art. In such methods, a hybrid RNA/DNA oligonucleotide (chimeroplast) is introduced into cells (WO 95/15972; Kren, Hepatology 25 (21997), 1462-1468; Cole-Stauss, Science 273 (1996), 1386-1389). Without being bound by theory, a part of the DNA component of the RNA/DNA oligonucleotide is thereby homologous to a nucleotide sequence occurring endogenously in the cell and encoding a corresponding protein, but displays a mutation or comprises a heterologous part which lies within the homologous region. Due to base pairing of the regions of the RNA/DNA oligonucleotide which are homologous to the endogenous sequence with these sequences, followed by homologous recombination, the mutation or the heterologous part contained in the DNA component of the oligonucleotide can be introduced into the cell genome. This leads to a reduction of the activity, i.e. expression, of the gene, into which the heterologous part or the mutation has been introduced.

In view of the above, it is clear that the nucleic acid molecule causing in vivo mutagenesis may comprise a heterologous sequence or a sequence carrying a mutation flanked by parts of a nucleotide sequence encoding a subunit of the oligomeric complex defined herein.

In a further particular embodiment of the invention, the “antagonist” to be employed is a nucleic acid molecule that leads to a reduction or depletion of the activity of the oligomeric complex defined herein by a cosuppression effect. “Cosuppression effect” means that the synthesis of a nucleotide sequence, particularly of an RNA, in a living cell reduces the expression of a gene being homologous to said nucleotide sequence. The general principle of cosuppression and corresponding methods are well known to the person skilled in the art and are described, for example, in Pal-Bhadra (Cell 90, 1997), 479-490) and Birchler (Nature Genetics 21 (1999), 148-149). In a particular embodiment, the nucleic acid molecule causing a cosuppression effect comprises a nucleotide sequence encoding a subunit of the oligomeric complex defined herein or a fragment of said nucleotide sequence.

In another specific embodiment of this invention an “agonist” is a molecule selected from the group consisting of:
(a) a polypeptide as defined herein above, for example a subunit of the herein defined oligomeric complex or said oligomeric complex itself, or a nucleotide sequence comprising a nucleic acid molecule as defined herein above, for example a nucleic acid molecule encoding a subunit of the herein defined oligomeric complex;
(b) a low molecular weight compound or a small molecule, for example being capable of enhancing the activity of the oligomeric complex comprising Afg3l1 and/or Afg3l2 or (a) variant(s) thereof as defined herein; and
(c) a binding molecule as defined herein, wherein said binding molecule is agonistic with respect to the activity of the oligomeric complex as defined and described herein (for example an agonistic antibody or agonistic aptamer).

Particularly, a low molecular weight compound or a small molecule as employed in context of this invention may be a compound/molecule having a molecular weight of less than about 2500 g/mol, preferably less than about 1500 g/mol, more preferably less than about 1000 g/mol and most preferably less than about 500 g/mol.

The skilled person is readily in the position to find out whether a certain binding molecule as defined herein is agonistic (for example an agonistic antibody or agonistic aptamer) or antagonistic (for example an antisense nucleotide sequence, siRNA or ribozyme)

Based on the findings provided herein, it is envisaged in one embodiment of this invention that particular such oligomeric complexes are administered, the subunit composition of which varies dependent on the tissue affected by the disease or disorder to be addressed, i.e. dependent on cell type specific mitochondrial defects. In other words, based on the teaching provided herein, the subunit composition of the oligomeric complex may be adjusted to (a) particular tissue(s) affected by a disorder or disease described herein.

It is envisaged herein that the compound to be administered in accordance with this invention may, optionally, comprise a pharmaceutically acceptable carrier and/or diluent.

Examples of suitable pharmaceutically acceptable carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. The resulting pharmaceutical compositions can be administered to the subject at a suitable dose, i.e. a dose leading to a pharmaceutically active amount of the compound to be employed/used herein at its desired site of effect.

Administration of the compound to be administered in accordance with the present invention may be effected by different ways, e.g., by intravenous, intraperitoneal, subcutaneous, intramuscular, topical, intradermal, intranasal or intrabronchial administration (for example as effected by inhalation) or by direct administration (for example injection) into a particular tissue or organ.

The dosage regimen of the compound to be administered in accordance with this invention will be determined by the attending physician and clinical factors. As it is well known in the medical arts, dosages for any one patient depends upon many factors, including the patient's size, body surface area, age, the particular compound to be administered, sex, time and route of administration, general health, and other drugs being administered concurrently. A person skilled in the art is aware of and is able to test the relevant doses, the compounds to be used in terms of the present invention are to be administered in.

In the context of the invention, it is of note that a preferred subject/patient in the context of the present invention is a mammalian subject/patient, more preferably a primate subject/patient, most preferably a human being, preferably in need of medical intervention, either in form of treatment, prevention and/or amelioration.

In a particular embodiment, the method for medical intervention provided, and hence the corresponding compound to be administered, are envisaged to be employed in context of gene therapy. This is particularly envisaged, when the “compound” as employed herein is or comprises (a) nucleic acid molecule(s) or is encoded by (a) nucleic acid molecule(s). For example, such corresponding nucleic acid molecule(s) may then be employed in form of an insert comprised in a vector, particularly in an expression vector. Such (expression) vector may particularly be a vector suitable for gene therapy approaches (for example a viral (expression) vector).

Gene therapy, which is based on introducing therapeutic genes into cells by ex-vivo or in-vivo techniques is one of the most important applications of gene transfer. Suitable vectors, methods or gene-delivering systems for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813, Isner, Lancet 348 (1996), 370-374; Muhlhauser, Circ. Res. 77 (1995), 1077-1086; Onodua, Blood 91 (1998), 30-36; Verzeletti, Hum. Gene Ther. 9 (1998), 2243-2251; Verma, Nature 389 (1997), 239-242; Anderson, Nature 392 (Supp. 1998), 25-30; Wang, Gene Therapy 4 (1997), 393-400; Wang, Nature Medicine 2 (1996), 714-716; WO 94/29469; WO 97/00957; U.S. Pat. No. 5,580,859; U.S. Pat. No. 5,589,466; U.S. Pat. No. 4,394,448 or Schaper, Current Opinion in Biotechnology 7 (1996), 635-640, and references cited therein.

The nucleic acid molecules and vectors may be designed for direct introduction or for introduction via liposomes, viral vectors (e.g. adenoviral, retroviral), electroporation, ballistic (e.g. gene gun) or other delivery systems into the cell. Additionally, a baculoviral system can be used as eukaryotic expression system for the above-defined nucleic acid molecules. The introduction and gene therapeutic approach should, preferably, lead to the expression of a functional “compound” in accordance with this invention (for example an antisense or siRNA construct), whereby said expressed “compound” is particularly useful in the treatment, amelioration and/or prevention of the diseases or disorders defined herein.

The term “vector” as used herein particularly refers to plasmids, cosmids, viruses, bacteriophages and other vectors commonly used in genetic engineering. In a preferred embodiment, the vectors of the invention are suitable for the transformation of cells, like fungal cells, cells of microorganisms such as yeast or bacterial cells or animal cells. As mentioned, in a particularly preferred embodiment such vectors are suitable for use in gene therapy.

In one aspect of the invention, the vector to be employed is suitable for stable transformation of an organism, and hence is an expression vector. Generally, expression vectors have been widely described in the literature. As a rule, they may not only contain a selection marker gene and a replication-origin ensuring replication in the host selected, but also a promoter, for example a promoter as defined herein, and in most cases a termination signal for transcription. Between the promoter and the termination signal there is in general at least one restriction site or a polylinker which enables the insertion of a nucleotide sequence desired to be expressed.

The DNA sequence naturally controlling the transcription of the corresponding gene/nucleic acid molecule, e.g. the promoter sequence of the LHR gene, can be used as the promoter sequence, if it is active in the selected host organism. However, this sequence can also be exchanged for other promoter sequences. It is possible to use promoters ensuring constitutive expression of the gene/nucleic acid molecule and inducible promoters which permit a deliberate control of the expression of the gene/nucleic acid molecule. Bacterial and viral promoter sequences possessing these properties are described in detail in the literature. Regulatory sequences for the expression in microorganisms (for instance E. coli, S. cerevisiae) are sufficiently described in the literature. Promoters permitting a particularly high expression of a downstream sequence are for instance the T7 promoter (Studier et al., Methods in Enzymology 185 (1990), 60-89), lacUV5, trp, trp-lacUV5 (DeBoer et al., in Rodriguez and Chamberlin (Eds), Promoters, Structure and Function; Praeger, N.Y., (1982), 462-481; DeBoer et al., Proc. Natl. Acad. Sci. USA (1983), 21-25), Ip1, rac (Boros et al., Gene 42 (1986), 97-100). Inducible promoters are preferably used for the synthesis of polypeptides. These promoters often lead to higher polypeptide yields than do constitutive promoters. In order to obtain an optimum amount of polypeptide, a two-stage process is often used. First, the host cells are cultured under optimum conditions up to a relatively high cell density. In the second step, transcription is induced depending on the type of promoter used. In this regard, a tac promoter is particularly suitable which can be induced by lactose or IPTG (=isopropyl-β-D-thiogalactopyranoside) (deBoer et al., Proc. Natl. Acad. Sci. USA 80 (1983), 21-25). Termination signals for transcription are also described in the literature.

Examples of vectors suitable to comprise the nucleic acid molecule(s) as employed in context of the present invention are known in the art.

For example, such vectors may be suitable for gene therapy, i.e. the vector of the present invention may also be a gene transfer and/or gene targeting vector. For gene therapy, various viral vectors which can be utilized are, for example, adenovirus, herpes virus, vaccinia, or, preferably, an RNA virus such as a retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can also incorporate multiple genes. All of these vectors can transfer or incorporate a gene for a selectable marker so that transduced cells can be identified and generated.

Retroviral vectors can be made target specific by inserting, for example, a polynucleotide encoding a sugar, a glycolipid, or a protein. Those of skill in the art will know of, or can readily ascertain without undue experimentation, specific polynucleotide sequences which can be inserted into the retroviral genome to allow target specific delivery of the retroviral vector containing the inserted polynucleotide sequence.

As mentioned, the meanings of terms like “OPA1”, “OPA1 processing” and “proteolytic cleavage of OPA1” are known in the art (Ishihara loc cit; Duvezin-Caubet loc cit) and can also be deduced from PCT/EP2007/004466 (EP-attorney docketing no.: M1590 PCT S3; claiming priority to U.S. 60/801,484). These known definitions apply in context of this invention, if not explicitly defined otherwise.

In view of this, “OPA1 processing” as defined herein is intended to be characterized by a certain amount of at least one large isoform of OPA1, a certain amount of at least one small isoform of OPA1 and/or a certain ratio of at least one large versus at least one small isoform of OPA1. Thereby, the OPA1 isoforms are formed by proteolytic cleavage of OPA1, i.e. of one or more of the OPA1 spliceforms. Usually, in mammalian cells, “OPA1 processing” usually occurs to a relatively moderate extent, referred to herein as “normal OPA1 processing” or simply “OPA1 processing”. In difference to this, “altered OPA1 processing” as defined herein is intended to be characterized by an altered amount of at least one large and/or at least one small isoform of OPA1 and/or an altered ratio of at least one large versus at least one small isoform of OPA1 (due to an altered proteolytic cleavage of OPA1) as compared to a control/standard. “Control/standard” in this context means a physiological condition, where “normal OPA1 processing” or simply “OPA1 processing” occurs (For example in healthy living cells, like the Hela cells or yeast WT cells employed herein).

Large isoform(s) of OPA1 as defined herein have an apparent molecular weight of more than about 91 kD and small isoform(s) as defined herein have an apparent molecular weight of less than about 91 kD, when said molecular weights being determined by SDS-PAGE analysis, in particular an 10% gel as disclosed herein and described in the appended examples.

It is evident for the person skilled in the art that also other SDS gels and means (in particular Western-Blot analysis and the like) are useful and envisaged in context of the present invention. It is of note that the herein given value of 91 kD is, accordingly, an illustrative example and the person skilled in the art can also use other means to deduce the identity, amount and/or ratio of the herein described OPA isoforms (e.g. the presence or absence of said OPA1-isoforms) in a given sample to be analysed. For example, said large OPA1 isoforms have an apparent molecular weight of more than about 95 kD or, preferably, of more than about 99 kD and the small OPA1 isoforms have an apparent molecular weight of less than about 95 kD or, preferably, of more than about 99 kD, when said molecular weights being determined by peptide analysis, e.g. mass spectrometry.

In context of the present invention, “OPA” or “OPA1” means the optic atrophy 1 protein/gene, in particular OPA1 of human origin. Yet, in certain embodiments it is also envisaged that OPA1 of other organisms, e.g. of mouse, rat, pig, dog, bovine species or fruit fly, be assessed in context of this invention. The nucleotide and amino acid sequences of human OPA1, particularly of the eight spliceforms of OPA1, are given in the appended sequence listing and examples.

It is of note that the nucleotide and amino acid sequences of OPA1 given herein below are not limiting. Accordingly, the term “OPA” or “OPA1” also encompasses OPA1 proteins/genes having amino acid or nucleotide sequences being derivatives of those given sequences.

In terms of the present invention the term “derivatives” or “derivatives thereof” or “variants” refers to amino acid or nucleotide sequences being homologous to the amino acid or nucleotide sequences shown herein, e.g. those of human OPA1, and/or amino acid or nucleotide sequences as shown herein, e.g. those of human OPA1, but having (a) particular (conservative) amino acid(s) exchanged. For instance, in context of the present invention, “homologous” means that amino acid or nucleotide sequences have identities of at least 50%, 60%, 70%, 80%, 90%, 95%, 98% or 99% to the sequences shown herein, e.g. those of human OPA1, wherein the higher identity values are preferred upon the lower ones.

As shown herein, upon drug-induced apoptosis, processing of OPA1 and mitochondrial fragmentation preceded cytochrome c release. When the mitochondrial membrane potential was dissipated, processing of OPA1 and fragmentation of mitochondria, but not cytochrome c release was observed. The same phenomenon was observed in cybrid cells from a patient with MERRF syndrome and in mouse embryonic fibroblasts harbouring an error-prone mitochondrial DNA (mtDNA) polymerase gamma, Furthermore, processed OPA1 was observed in heart tissue derived from heart-specific TFAM −/− knockout mice suffering from mitochondrial cardiomyopathy and in skeletal muscle from patients with mitochondrial disorders such as MELAS. Processing of OPA1 was inhibited by addition of ortho-phenanthroline and partially by addition of DCI (3,4-Dichloroisocoumarin) in vivo.

Recovery of mitochondrial fusion was accompanied by resynthesis of large isoforms of OPA1. Fragmentation of mitochondria could be prevented by overexpressing OPA1.

This demonstrates that various forms of mitochondrial dysfunction lead to proteolytic processing of OPA1 resulting in impaired mitochondrial fusion and points out to the existence of a pathway in which processing of OPA1 leads to fragmentation of mitochondria. Without being bound by theory, this fragmentation then serves as an early response in order to segregate dysfunctional mitochondria from the network of functional mitochondria.

These findings demonstrate a proteolytic processing of larger OPA1 isoforms into smaller ones and a corresponding relationship to mitochondrial dysfunction. Accordingly, said processing occurs under various conditions of mitochondrial dysfunction, said dysfunction being linked to a pathological status.

The processes of mitochondrial damage leading to dysfunction, breakdown of mitochondrial bioenergetic competence and mitochondrial fragmentation are linked through a cascade of reactions. In context of the present invention, inter alia, a central molecular player, namely OPA1, that links changes in mitochondrial morphology with mitochondrial dysfunction was identified. This result is exemplary based on a wide variety of established model systems and patient material for, e.g., MERRF, MELAS, mtDNA depletion syndrome, dilated cardiomyopathy, diseases with respiratory deficiencies, e.g. diseases with respiratory deficiencies of unknown origin and aging.

Without being bound by theory, it can be deduced how this cascade is organized: Mitochondrial dysfunction leads to impairment of bioenergetic competence of mitochondria. This results in reduced membrane potential and ATP production. In such compromised mitochondria a proteolytic processing of large to small isoforms of OPA1 is activated. As a consequence, fusion of mitochondria is blocked and dysfunctional mitochondria are segregated from the network of intact mitochondria. This in turn triggers further reactions such as removal and degradation of the dysfunctional fragments as reported in several systems (Priault, 2005, Cell Death Differ, online publication, 10 Jun. 2005; Lyamzaev, 2004, Biochem Soc Trans 32, 1070; Skulachev, 2004, Mol Cell Biochem 256-257, 341). A key element of this mechanism is the regulatory inactivation of fusion-promoting OPA1 by proteolytic cleavage.

It was found in the art that, in contrast to OPA1, in the yeast homolog of OPA1 (Mgm1), lack of either the large or the small isoforms leads to an impairment of mitochondrial fusion (Herlan, 2003, J Biol Chem 278, 27781; Herlan, 2004, J Cell Biol 165, 167; Sesaki, 2003, Biochem Biophys Res Commun 308, 276). Accordingly, it was speculated, that a proper balance of long and short isoforms of Mgm1 is critical for maintainance of tubular mitochondrial morphology and that, in analogy thereto, there is also the possibility that an overexpression of OPA1 can lead to an imbalance in OPA1 isoforms (Chen, 2005, J Biol Chem 280, 26185)

Yet, based on the teaching provided in context of this invention, mitochondrial dysfunction (or a corresponding mitochondrial disease or disorder) is not merely correlated with decrease of any one of OPA1 isoforms, but with a decrease of particularly the large isoforms, e.g. OPA1#1 (as defined herein) and OPA1#2 (as defined herein), accompanied by an increase of the small isoforms, e.g. OPA1#3 (as defined herein) and OPA1#5 (as defined herein).

In context of the present invention it was, inter alia, found that the described large and small isoforms of yeast Mgm1 do not correlate to large and small isoforms of OPA1. Accordingly, the above mentioned finding that mitochondrial fragmentation in humans is correlated with a decrease of particularly large isoforms of OPA1 and an an increase of particularly small isoforms of OPA1 was unexpected.

In yeast, mitochondrial dysfunction causes a deficiency in the import of the Mgm1 precursor. Consequently, formation of the small isoform and mitochondrial fusion are impaired (Herlan, 2004, J Cell Biol 165, 167). In humans, mitochondrial dysfunction is sensed in a different fashion which is independent of protein synthesis and consequently protein import into mitochondria. Still, this leads to the specific, rapid, and intramitochondrial inactivation of the orthologous effector protein, OPA1.

Taken together, mitochondrial dysfunction leads to or comes along with a rapid conversion of the large isoforms into the small isoforms in humans whereas in yeast this process occurs in the opposite direction (increase of large isoform) and is slow since it requires protein turnover.

Satoh (2003, Biochem Biophys Res Commun, 300, 482-493) identified 2 OPA1 isoforms in HeLa cells and speculates that their differential association with the inner and outer mitochondrial membrane suggests the different roles of each of these proteins for controlling the mitochondrial morphology. It is not evident from Satoh that a specific pattern of occurrence of different OPA1 isoforms, not to mention the herein described decrease of large accompanied with an increase of small isoforms, correlates with the fragmentation status of mitochondria.

In context of the present invention, the term “about”, with respect to certain given molecular weight values, means +/−3 kD, preferably +/−2 kD, more preferably +/−1 kD, more preferably +/−0.5 kD and most preferably +/−0.1 kD. Moreover, in context of the present invention, it is envisaged that the term “less than about xx kD”, for example “less than about 91 kD”, “less than about 95 kD” or “less than about 99 kD”, also comprises molecular weight values being equal to xx kD, for example equal to 91, 95 kD or 99 kD.

It is evident for the person skilled in the art that certain given molecular weight values may vary, dependent on the preparational/experimental conditions employed, or, for example with respect to mass spectrometry, dependent on the information content resulting from the preparational/experimental method employed or dependent on an employed modification of the proteins/peptides to be analyzed due to a specific preparational/experimental procedure. It is, for example, known in the art that proteins/peptides to be analyzed via mass spectrometry can be modified, i.e. their theoretical molecular weight can be increased (e.g. by certain chemical modifications) or decreased (e.g. by using (a) certain protease(s)) by a certain value. It is therefore of note in context of the present invention that the molecular weight values given for certain OPA1 isoforms can change, dependent on the particular preparational/experimental conditions employed during the corresponding mass spectrometry experiment (or other methods for determining molecular weights). The skilled person is readily in the position to deduce whether certain changes/differences of given molecular weight values result from the particular preparational/experimental method employed or form a specific composition of the protein/peptide analysed.

In context of the present invention, the term “isoform” of OPA1 means a certain form of the OPA1 protein. Without bound by theory, an OPA1 isoform derives from (a protein encoded by) any one of spliceforms 1 to 8 of OPA1, e.g. by posttranslational processing (e.g. proteolytical processing). Without bound by theory, said posttranslational processing (e.g. proteolytical processing) leads to a shortened N-terminus of OPA1, particularly of the spliceforms thereof, wherein the C-terminus remains complete. The “isoforms” of OPA1 to be scrutinized in context of the present invention are described herein in more detail. Accordingly, the term “corresponding” in context of OPA1 isoforms and OPA1 spliceforms, e.g. in the term “an OPA1 isoform having an apparent molecular weight calculated from amino acid sequences of the corresponding spliceform(s)”, means that the respective OPA1 isoform can be related to or may be derived from said OPA1 spliceform(s). These spliceforms are also described herein below.

In context of the present invention, the term “spliceform” or “splice variant” of OPA1 means a form of OPA1 that emerges by alternative splicing of the primary transcript transcribed from the OPA1 gene. It is envisaged herein, that the term “spliceform” either refers to the mature transcript generated by alternative splicing, but also refers to the corresponding protein which has been translated from said mature transcript. Accordingly, the term “isoform being derived from (corresponding) spliceform” means that an OPA1 isoform originates from a protein that has been translated from a mature (alternatively spliced) transcript of the OPA1 gene. Thereby, posttranslational processing (e.g. proteolytical processing) of said protein that has been translated from a mature (alternatively spliced) transcript of the OPA1 gene may occur. However, an OPA1 isoform may also directly originate from said protein, without further posttranslational processing. In such specific case, said protein then is said OPA1 isoform.

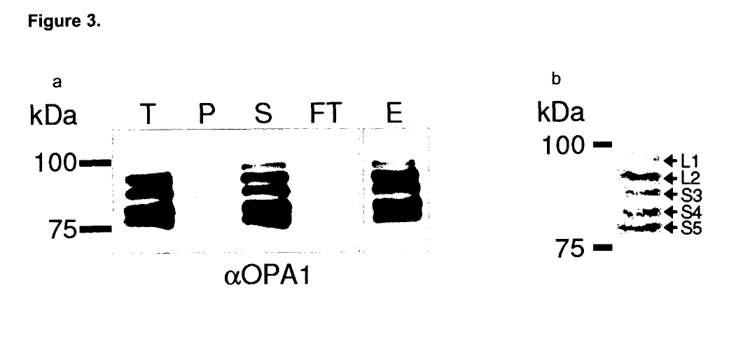

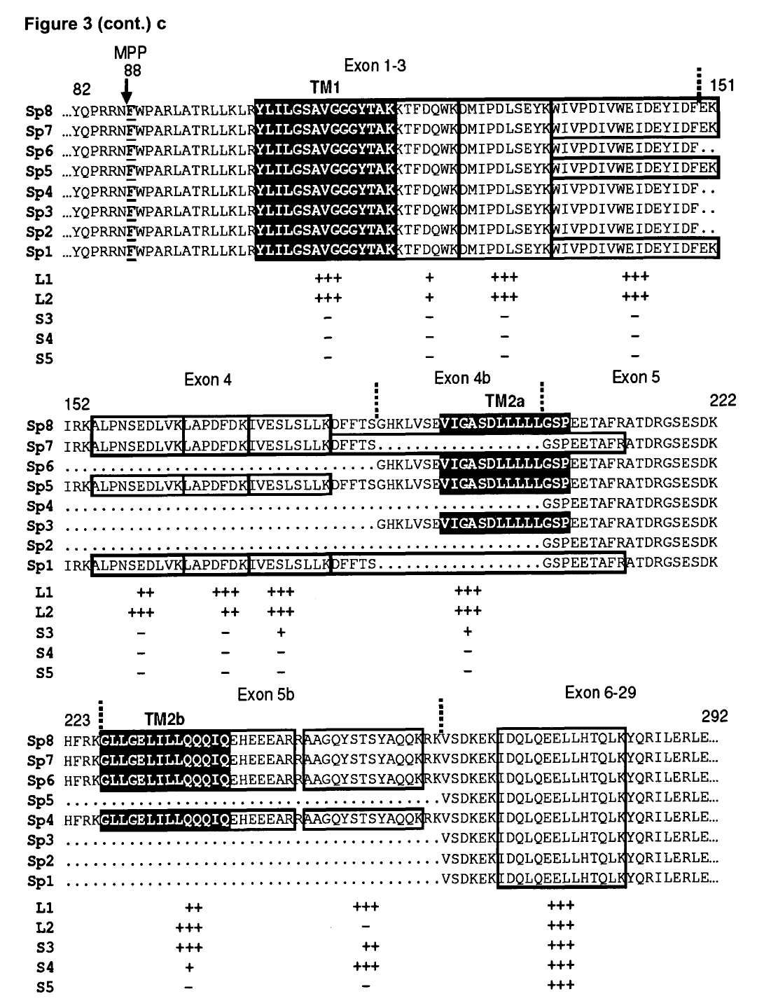

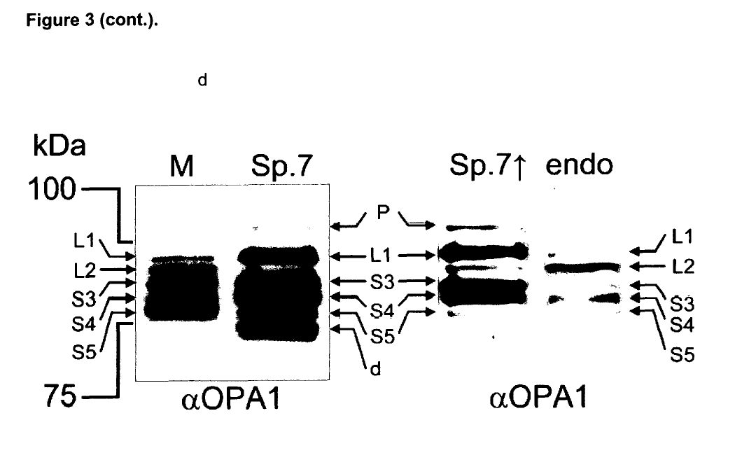

At present, 8 spliceforms of OPA1 are known in the art, which emerge by alternative splicing of exon 4, exon 4b and/or exon 5 (see FIG. 3c). The corresponding amino acid sequences of these 8 spliceforms are given in SEQ ID No: 2, 4, 6, 8, 10, 12, 14 and 14 and are partially shown in FIG. 3c. Their corresponding nucleotide acid sequences are given in SEQ ID No: 1, 3, 5, 7, 9, 11, 13 and 15.

Dependent whether exon 4, exon 4b and/or exon 5 is comprised, the OPA1 spliceforms can be defined by specific amino acid sequences, e.g. by one of the following amino acid sequences:

EYKWIVPDIVWEIDEYIDFGHKLVSEVIGASDLLLLL (SEQ ID NO: 31) corresponds to the amino acid sequences from exon 3 to exon 4b (lack of exon 4) and is comprised in spliceforms 3 and 6.

EYKWIVPDIVWEIDEYIDFGSPEETAFRATDRGSESDKHFRK (SEQ ID NO: 32) corresponds to the amino acid sequences from exon 3 to exon 5 (lack of exon 4 and 4b) and is comprised in spliceforms 2 and 4.

EKIRKALPNSEDLVKLAPDFDKIVESLSLLKDFFTSGSPEETAFRATDRGSESDKHFR K (SEQ ID NO: 33) corresponds to the amino acid sequences from exon 4 to exon 5 (lack of exon 4b) and is comprised in spliceforms 1 and 7.

GSPEETAFRATDRGSESDKHFRKVSDKEKIDQLQEELLHTQLKYQRILERLEKENKE LRK (SEQ ID NO: 34) corresponds to the amino acid sequences from exon 5 to exon 6 (lack of exon 5b) and is comprised in spliceforms 1, 2, 3 and 5.

Other amino acid sequences specific for a certain OPA1 spliceform can be derived from the amino acid sequences of the OPA1 spliceforms given herein below.

Since an OPA1 isoform to be employed in context of the present invention may be derive from one particular OPA1 spliceform, the above mentioned amino acid sequences defining the different OPA1 spliceforms may also be used to determine the identity, amount and/or ratio (e.g. the presence or absence) of a given OPA1 isoform as defined herein. For example, since the present invention provides evidence that OPA1-L1 be derived from spliceform 7 and OPA1-L2 be derived from spliceform 1, OPA1-L1 may, e.g. be characterized in that it comprises the amino acid sequence EKIRKALPNSEDLVKLAPDFDKIVESLSLLKDFFTSGSPEETAFRATD RGSESDKHFRK (SEQ ID NO: 33) and in that it not comprises the amino acid sequence GSPEETAFRATDRGSESDKHFRKVSDKEKIDQLQEELLHTQLKYQRILER LEKENKELRK (SEQ ID NO: 34) and OPA1-L2 may, e.g. be characterized in that it comprises the amino acid sequence EKIRKALPNSEDLVKLAPDFDKIVESLSLLKDFF TSGSPEETAFRATDRGSESDKHFRK (SEQ ID NO: 33) and GSPEETAFRATDRGS ESDKHFRKVSDKEKIDQLQEELLHTQLKYQRILERLEKENKELRK (SEQ ID NO: 34). However, since the OPA1 isoforms to be employed in context of the present invention may derive from the OPA1 splicefroms by (proteolytical) processing, not the complete amino acid sequences as given above, but fragments or derivatives thereof, may be used to determine a certain OPA1 isoform.

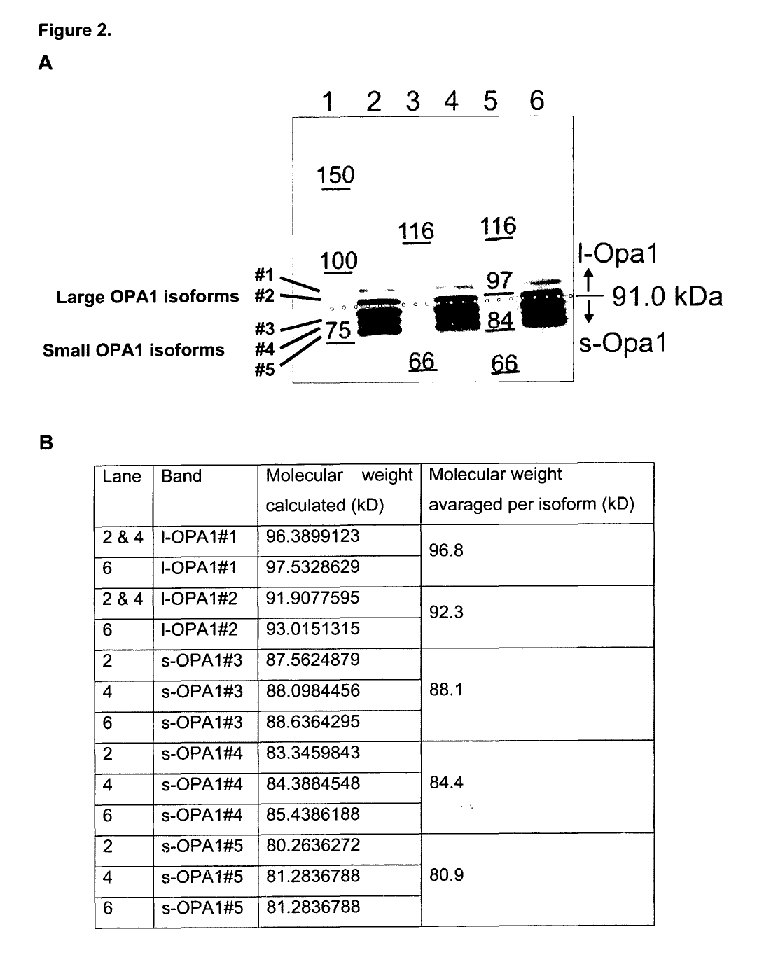

Particularly useful in context of the present invention, and exemplified in the appended examples (e.g. FIG. 2), are SDS-PAGE-gels having a polyacrylamide concentration of 10%. However, it is also envisaged that SDS-PAGE-gels to be employed in context of the present invention have other polyacrylamide concentrations. E.g. said concentrations may be 1%, 2%, 3%, 4%, 5% or 10% higher or lower than that of a 10% SDS-PAGE, e.g. than that of the 10% SDS-PAGE-gel as exemplified herein, but also other concentrations are envisaged. A person skilled in the art is readily in the position to transfer molecular weight values deduced on the basis of an SDS-PAGE-gel having a certain polyacrylamide concentration, e.g. 10% as exemplified herein (e.g. FIG. 2), to molecular weight values deduced on the basis of an SDS-PAGE-gel having a different polyacrylamide concentration. As mentioned above, also other means than direct determination of a given molecular weight from an SDS-PAGE-gel may be used in context of this invention. For example, the appended experimental data provide for corresponding Western-blots.

The meaning of the term “Mass spectrometry” (MS) is and corresponding methods are known in the art. Particularly useful “mass spectrometry” methods to be employed in context of the present invention, and as exemplified herein (Example 3), are MALDI-MS or LC-MS/MS. Further “mass spectrometry” methods are known in the art and can easily be adapted to the specific needs of the present invention by a person skilled in the art.

The term “molecular weights being determined by mass spectrometry” means that the apparent molecular weight of a certain OPA1 isoform is determined by performing mass spectrometry analysis on said OPA1 isoform (e.g. as in example 3) and using the results of said mass spectrometry analysis to calculate said apparent molecular weight of said certain OPA1 isoform on the basis of the amino acid sequence of OPA1. Since eight alternative spliceforms exist of OPA1, having different amino acid sequences, the result of said calculation may vary, dependent on the spliceform, the amino acid sequence of which is used for said calculation. Examples of such determination of molecular weights by mass spectrometry are given herein (example 3). The principle of such determination is described in the following:

First, the amino acid sequences of certain peptide stretches comprised in an OPA1 isoform to be analysed is determined by mass spectrometry (e.g. as in example 3). Second, the resulting amino acid sequences of said peptide stretches are then compared with the amino acid sequences of the eight spliceforms of OPA1. Then, it is determined which certain OPA1 spliceforms comprise the amino acid sequences of said peptide stretches and which do not. Subsequently, of the spliceforms that comprise the amino acid sequences of said peptide stretches, that amino acid sequence it is estimated, which starts with the amino acid sequence of the most N-terminal peptide determined. Last, from said estimated amino acid sequence, the theoretical molecular weight of the respective OPA1 isoform to be analysed is calculated based on the known molecular weights of the amino acid residues comprised.

It is of note that the so determined theoretical molecular weight may be further increased by the presence of a few further N-terminally located amino acid residues present in the (proteolytically) processed mature OPA1 isoform. The person skilled in the art is readily in the position to determine said slightly increased molecular weight, by taking advantage of the teaching of the present invention. As a non limiting example, the determination of such slightly increased theoretical molecular weight of certain OPA1 isoforms is exemplarily demonstrated in appended example 3

In context of the present invention, the large isoforms of OPA1 may comprise two isoforms (e.g. OPA1-L1 and OPA1-L2) and the small isoforms of OPA1 may comprise three isoforms (e.g. OPA1-S3, OPA1-S4 and OPA1-S5). However, it is also envisaged that further, possibly existing isoforms may assigned as large or small isoforms in context of the present invention. For instance, it is evident for a skilled person that, e.g., single bands of an SDS-PAGE/Western-blot as exemplified herein, may represent not only one, but several different isoforms and/or that further isoforms, larger or smaller than the particular isoforms defined herein may be present. For example, particularly the band corresponding to OPA1-S4 as defined herein may correspond to (a) further OPA1 isoform(s). Again, the gist of the present invention is based on the fact the determination of “small” versus “large” isoforms is illustrative for mitochondrial dysfunction and corresponding related disorders/diseases. Therefore, further, possibly existing isoforms may, e.g., be detectable by alternative comparable methods known in the art and may also be taken into consideration in the herein provided methods and means. For example, such methods may be SDS-PAGEs taking advantage of gels having very low polyacrylamide concentrations (e.g. 1%, 2%, 3% or 4%) and/or Western-blots taking advantage of radionuclide labelling, e.g. radionucleotide labelling of (secondary) antibodies used in said Western-blots, or other labelling approaches known in the art, e.g. other very sensitive labelling approaches being suitable for the detection of proteins being present in low amount(s)/concentration(s). Moreover, such methods may be a two dimensional gelelectrophoresis methods. These and other alternative methods for detecting isoforms of certain proteins/genes, like OPA1, are known in the art. It is envisaged that such alternative methods may also be employed in context of the present invention.

However, it is preferred that each single band as evident from the SDS-PAGE analysis as employed and exemplified herein represents one single OPA1 isoform. Accordingly, in one embodiment of the present invention, the two large OPA1 isoforms as defined herein (e.g. OPA1-L1 and OPA1-L2) are represented by two single bands, and the three small OPA1 isoforms as defined herein (e.g. OPA1-S3, OPA1-S4 and OPA1-S5) are represented by three single bands occurring in an SDS-PAGE, e.g. an SDS-PAGE as exemplified herein.

In context of the present invention, the two large OPA1 isoforms are indicated by numbers 1 and 2, namely 1 for the largest and 2 for the second largest OPA1 isoform. The three small OPA1 isoforms are indicated by numbers 3, 4 and 5, namely 3 for the largest of the three small isoforms, 4 for the second largest of the three small isoforms and 5 for the smallest isoform. The numbering of the OPA1 isoforms to be employed in context of the present invention is also given in the appended examples and corresponding figures, e.g. FIG. 2. In accordance thereto the OPA1 isoforms as employed in context of the present invention are termed as follows: OPA1-L/l1, L/l-OPA1#1, OPA1#1 or L/l1-OPA1 for the largest OPA1 isoform. OPA1-L/l2, L/l-OPA1#2, OPA1#2 or L/l2-OPA1 for the second largest OPA1 isoform. Large isoform(s) in context of the present invention is (are), e.g., OPA1-L1 and/or OPA1-L2. OPA1-S/s3, S/s-OPA1#3, OPA1#3 or S/s3-OPA1 for the largest of the three small OPA1 isoforms. OPA1-S/s4, S/s-OPA1#4, OPA1#4 or S/s4-OPA1 for the second largest of the three small isoforms. OPA1-S/s5, S/s-OPA1#5, OPA1#5 or S/s5-OPA1 for the smallest OPA1 isoform. Accordingly, small isoform(s) in context of the present invention is (are), e.g., OPA1-S3, OPA1-S4 and/or OPA1-S5.

It is of note that the specific numbering is indicative for the specific OPA1 isoform, and that the additional terming, like “l” for large; “s” for small or “OPA”, “OPA1” or “OPA1#” for OPA1 may slightly vary. However the abbreviations “L” or “l” indicate large isoforms and “S” or “s” indicate small isoforms of OPA1.

In view of the teaching provided herein, also in the appended examples, the OPA1 isoforms employed in context of the present invention are defined as follows:

In one aspect of the present invention, the term “OPA1 isoform” means a protein encoded by the OPA1 gene, but particularly be derived from at least one of the different spliceforms of OPA1 (e.g. from at least one of spliceforms 1 to 8 as partially depicted in FIG. 3c), e.g. by posttranslational (e.g. proteolytical) processing, wherein said proteins are distinguishable by their molecular weight and/or (a) certain amino acid sequence(s). From the above, it is, inter alia, evident that an “OPA1 isoform” as employed in context of the present invention comprises (an) amino acid stretche(s) which unambiguously characterize it as a polypeptide/protein derived from OPA1. In this context, “derived from OPA1” particularly means encoded by the OPA1 gene and/or generated from OPA1 by the herein described and defined OPA1 processing. Thus, an “OPA1 isoform” as employed can particularly be characterized by (a) certain amino acid stretch(es) of any one of SEQ ID No: 2, 4, 6, 8, 10, 12, 14 or 16 or by (a) certain amino acid stretch(es) encoded by any one of SEQ ID No: 1, 3, 5, 7, 9, 11, 13 or 15.

In context of the present invention, the term “molecular weight” may, inter alia, refer to the apparent molecular weight. Said apparent molecular weight can be determined by methods known in the art. E.g., said apparent molecular weight can be determined by SDS-PAGE, and, accordingly, also from Western-blots, or can be calculated from the amino acid sequence of OPA1, particularly from the amino acid sequence(s) of the corresponding spliceform(s) by taking advantage of mass spectrometry methods. Examples of the determination of the OPA1 isoforms by using these techniques are given in the appended examples (e.g. example 2/3/11; FIG. 2/3).

As already mentioned above, in context of the present invention, certain given molecular weight values are apparent molecular weight values. It is envisaged, that the certain molecular weight values given herein may slightly vary, e.g. with respect to the molecular weight of the protein present in vivo. Said variation may by in the range of 5 kD, 4 kD, 3 kD, 2 kD, 1 kD, 0.5 kD, 0.4 kD, 0.3 kD, 0.2 kD or 0.1 kD, whereby the smaller variations are preferred over the larger variations. The definitions given for the term “about” with respect to molecular weight values herein above, apply here, mutatis mutandis.

In context of the present invention, large isoforms comprise an isoform having an apparent molecular weight of about 97 kD (96.8 kD) (defined as OPA1-L1) or an isoform having an apparent molecular weight of about 92 kD (92.3 kD) (defined as OPA1-L2), said molecular weights being determined by SDS-PAGE analysis. Moreover, in context of the present invention large isoforms comprise an isoform having an apparent molecular weight of about 104 kD (104.0 kD) or, preferably, of about 105 kD (105.1 kD) (defined as OPA1-L1) or an isoform having an apparent molecular weight of about 99 kD (99.2 kD) or, preferably, of about 100 kD (100.0 kD) (defined as OPA1-L2), said molecular weights being determined by mass spectrometry.

The molecular weight values determined by mass spectrometry of OPA1-L1 and OPA1-L2 are given as averaged values of the four smallest (OPA1-L2) and the four largest (OPA1-L1) molecular weight values of the second column (“l-OPA1#1/#2”) of the corresponding table in example 3.

In this context, it is of note that the present invention provides evidence that OPA1-L1 be derived from spliceform 7 and OPA1-L2 be derived from spliceform 1 or spliceform 4. Accordingly, it is particularly envisaged herein that OPA1-L1 has an apparent molecular weight determined by mass spectrometry of about 105 kD (104.9 kD) or, preferably, of about 106 kD (105.8 kD). OPA1-L2 is particularly envisaged to have an apparent molecular weight determined by mass spectrometry of about 101 kD (100.7 kD) or, preferably, of about 102 kD (101.5 kD), if determined on the basis of spliceform 1. If determined on the basis of spliceform 4, OPA1-L2 is particularly envisaged to have an apparent molecular weight determined by mass spectrometry of about 101 kD (100.8 kD) or, preferably, of about 102 kD (101.7 kD) (see second column (“l-OPA1#1/#2”) of the tables in example 3).

In addition to the above, the large OPA1 isoforms to be scrutinized in context of the present invention, namely OPA1-L1 and OPA1-L2, are characterized by comprising, e.g. as most N-terminal peptides, amino acid stretches or amino acid peptides comprising one or more of the following sequences: YLILGSAVGGGYTAK (SEQ ID No: 17), TFDQWK (SEQ ID No: 18), DMIPDLSEYK (SEQ ID No: 19), WIVPDIVWEIDEYIDFEK (SEQ ID No: 20), LAPDFDK (SEQ ID No: 21), IVESLSLLK (SEQ ID No: 22), ALPNSEDLVK (SEQ ID No: 23), DFFTSGSPEETAFR (SEQ ID No: 24) and TRLLKLRYLILGS (SEQ ID No: 25) and FWPARLATRLLKLRYLILGS (SEQ ID NO: 35), or derivatives thereof.

It is evident from the teaching provided herein that the large OPA1 isoforms to be scrutinized in context of the present invention, namely OPA1-L1 and OPA1-L2, may (further) be characterized by further amino acid stretches or amino acid peptides of OPA1, or of particular OPA1 spliceforms, e.g. by further amino acid stretches or amino acid peptides of OPA1, or of particular the OPA1 spliceforms, lying more C-terminal to the above mentioned amino acid stretches or amino acid peptides. For instance, said further amino acid stretches or amino acid peptides of OPA1 may, in addition to or instead of the above mentioned sequences, comprise one or more of the following sequences: GLLGELILLQQQIQEHEEEAR (SEQ ID No: 26), AAGQYSTSYAQQK (SEQ ID No: 27) or IDQLQEELLHTQLK (SEQ ID No: 28), or derivatives thereof. However, it is of note that particularly OPA1-L2 does not comprise amino acid stretches or amino acid peptides comprising one or more of the following sequences: GLLGELILLQQQIQEHEEEAR (SEQ ID No: 26) or AAGQYSTSYAQQK (SEQ ID No: 27), or derivatives thereof.

It is further evident from the teaching provided herein that, the large OPA1 isoforms to be scrutinized in context of the present invention, namely OPA1-L1 and OPA1-L2, may also be characterized by not comprising amino acid stretches or amino acid peptides comprising amino acid stretches of OPA1, particularly of the OPA1 spliceforms, lying more N-terminal to those amino acid peptides comprising amino acid stretches of OPA1, particularly of OPA1 spliceforms, that correspond to the most N-terminal peptides of the large OPA1 isoforms as defined herein, e.g. the peptide TRLLKLRYLILGS (SEQ ID No: 25) (see, e.g. Example 3; FIG. 3).

Accordingly, the large OPA1 isoforms to be scrutinized in context of the present invention, namely OPA1-L1 and OPA1-L2, are characterized by the feature that their apparent N-terminus correlates to amino acid position 102, preferably to amino acid position 95 of spliceforms 1 to 8 (SEQ ID No: 16, 14, 12, 10, 8, 6, 4 and 2, respectively).

As mentioned above, evidence is provided herein, that the OPA1 isoform OPA1-L1 be derived from spliceform 7 (SEQ ID Nos: 3/4) and the OPA1 isoform OPA1-L2 be derived from spliceform 1 (SEQ ID Nos: 15/16) or spliceform 4 (SEQ ID Nos: 9/10) of OPA1.

In context of the present invention, small isoforms comprise an isoform having an apparent molecular weight of about 88 kD (88.1 kD) (defined as OPA1-S3), an isoform having an apparent molecular weight of about 84 kD (84.4 kD) (defined as OPA1-S4) or an isoform having an apparent molecular weight of about 81 kD (80.9 kD) (defined as OPA1-S5), said molecular weights being determined by SDS-PAGE analysis. Moreover, in context of the present invention small isoforms comprise an isoform having an apparent molecular weight of about 92 kD (91.8 kD) or, preferably, of about 96 kD (95.9 kD) (defined as OPA1-S3), an isoform having an apparent molecular weight of about 89 kD (89.2 kD) or, preferably, of about 92 kD (91.8 kD) (defined as OPA1-S4) or an isoform having an apparent molecular weight of about 87 kD (86.8 kD) or, preferably, of about 87 kD (86.8 kD) (defined as OPA1-S5), said molecular weights being determined by mass spectrometry.

OPA1-S3 is additionally characterized by comprising, e.g. as most N-terminal peptides, amino acid stretches or amino acid peptides comprising one or more of the following sequences: IVESLSLLK (SEQ ID No: 22), DFFTSGSPEETAFR (SEQ ID No: 24), GLLGELILLQQQIQEHEEEAR (SEQ ID No: 26), AAGQYSTSYAQQK (SEQ ID No: 27) or IDQLQEELLHTQLK (SEQ ID No: 28), or derivatives thereof. OPA1-S4 is characterized by comprising, e.g. as most N-terminal peptides, amino acid stretches or amino acid peptides comprising one or more of the following sequences: GLLGELILLQQQIQEHEEEAR (SEQ ID No: 26), AAGQYSTSYAQQK (SEQ ID No: 27) or IDQLQEELLHTQLK (SEQ ID No: 28), or derivatives thereof. OPA1-S5 is characterized by comprising, e.g. as most N-terminal peptides, amino acid stretches or amino acid peptides comprising the following sequence: IDQLQEELLHTQLK (SEQ ID No: 28), or derivatives thereof.

Moreover, the small OPA1 isoforms to be scrutinized in context of this invention, namely OPA1-S3, OPA1-S4 and OPA1-S5, are characterized by not comprising amino acid stretches or amino acid peptides comprising one or more of the following sequences: YLILGSAVGGGYTAK (SEQ ID No: 17), TFDQWK (SEQ ID No: 18), DMIPDLSEYK (SEQ ID No: 19), WIVPDIVWEIDEYIDFEK (SEQ ID No: 20), LAPDFDK (SEQ ID No: 21), ALPNSEDLVK (SEQ ID No: 23) and TRLLKLRYLILGS (SEQ ID No: 25), or derivatives thereof. It is of note that the small OPA1 isoforms to be scrutinized in context of this invention, namely OPA1-S3, OPA1-S4 and OPA1-S5, are further characterized by not comprising amino acid stretches or amino acid peptides of OPA1, particularly of one of the OPA1 spliceforms, lying more N-terminal to those amino acid peptides comprising amino acid stretches of OPA1, particularly of OPA1 spliceforms, that correspond to the most N-terminal peptides of the small OPA1 isoforms as defined herein (see, e.g. Example 3; FIG. 3).

Furthermore, OPA1-S5 is characterized by not comprising amino acid stretches or amino acid peptides comprising one or more of the following sequences: GLLGELILLQQQIQEHEEEAR (SEQ ID No: 26) and AAGQYSTSYAQQK (SEQ ID No: 27).

Accordingly, OPA1-S3 is, inter alia, characterized by the feature that its apparent N-terminus correlates to amino acid position 172 of spliceform 7 (SEQ ID No: 4.

Furthermore, OPA1-S4 is characterized by the feature that its apparent N-terminus correlates to amino acid position 227 of spliceform 8 (SEQ ID No: 2), to amino acid position 209 of spliceform 7 (SEQ ID No: 4), to amino acid position 191 of spliceform 6 (SEQ ID No: 6) or to amino acid position 173 of spliceform 4 (SEQ ID No: 10). Moreover, OPA1-S5 is characterized by the feature that its apparent N-terminus correlates to amino acid position 270 of spliceform 8 (SEQ ID No: 2), to amino acid position 252 of spliceform 7 (SEQ ID No: 4), to amino acid position 234 of spliceform 6 (SEQ ID No: 6), to amino acid position 233 of spliceform 5 (SEQ ID No: 8), to amino acid position 216 of spliceform 4 (SEQ ID No: 10), to amino acid position 197 of spliceform 3 (SEQ ID No: 12), to amino acid position 179 of spliceform 2 (SEQ ID No: 14) or to amino acid position 215 of spliceform 1 (SEQ ID No: 16).

It is of note that the molecular weight values of the OPA1 isoforms scrutinized herein which were determined by SDS-PAGE analysis, are given as averaged values corresponding to the molecular weight values of three different isoform bands within an SDS-PAGE/Western-blot (see FIG. 2).

In context of the present invention it is to be understood that the term “most N-terminal peptide(s)” means (a) peptide(s), e.g. (a) peptide(s) as defined above, that lies in the N-terminal region of the corresponding OPA1 isoform. Preferably, said peptide(s) is (are) the indeed most N-terminal peptide(s), what means that, e.g. when taking advantage of a particular protease during mass spectrometry analysis (e.g. see example 3), no other peptide can be found in the corresponding OPA1 isoform lying in a more N-terminal direction of said peptide. However, it is of note that the N-terminal amino acid(s) of said “most N-terminal peptide(s)” may not be the most N-terminal amino acid(s) of the corresponding OPA1 isoform(s). The most N-terminal amino acid(s) of the corresponding OPA1 isoform(s) may also correspond to a slightly more N-terminal amino acid position of OPA1, particularly of the OPA1 spliceforms. For instance, said slightly more N-terminal amino acid position may be a position lying 1 to 15, preferably 1 to 10, more preferably 1 to 5, more preferably 1 to 4, more preferably 1 to 3, more preferably 1 to 2 or 1 amino acid position(s) in the N-terminal direction of the amino acid sequence(s) corresponding to the “most N-terminal peptide(s)”, e.g. the peptides as defined above.

Since, without being bound by theory, the particular amino acid sequence(s) of the “most N-terminal peptide(s)” as defined herein, e.g., depend(s) on the protease to be employed during mass spectrometry analysis (e.g. see example 3), the amino acid sequence(s) of the “most N-terminal peptide(s)” may slightly vary, e.g. in the above defined ranges in the N-terminal direction (and/or C-terminal direction). The person skilled in the art is readily in the position to figure out said slightly varied amino acid sequence(s) of the “most N-terminal peptide(s)”, and accordingly the N-terminal amino acid of the OPA1 isoforms, by taking advantage of the teaching of the present invention, and, e.g., by applying mass spectrometry analysis taking advantage of different proteases, which, e.g. may cut the amino acid strand at different positions. Such mass spectrometry analysis are also envisaged to be employed in context of the present invention.

In view of the above, it is also of note that the amino acid positions of the different OPA1 spliceforms that correlates to the apparent N-termini of the corresponding OPA1 isoforms given herein above, may slightly vary. For instance, this variation may in the range of 1 to 15 amino acids in the N-terminal direction, wherein the smaller variations within this range are preferred.

The term “derivatives” or “derivatives thereof” as well as “homologous” as defined herein above, also apply, mutatis mutandis, in context of the peptides shown above, e.g. the peptides comprised in the OPA1 isoforms or the peptides that characterize the OPA1 spliceforms. Moreover, the term “derivatives” or “derivatives thereof” also refers to (a) fragment(s), e.g. (a) fragment(s) of the peptides shown above, e.g. the peptides comprised in the OPA1 isoforms or the peptides that characterize the OPA1 spliceforms. The term “fragment(s)” means amino acid stretches of at least 3, 4, 5, 6, 7, 8, 9, 10, 11, 12, 13, 14, 15, 20, 30, 50, 100 or 150 amino acids. Also amino acid stretches having other numbers of amino acids are envisaged.

In terms of the present invention the term “derivatives” or “derivatives thereof” also comprises homologies as well as conservative amino acid exchanges and further known modifications.

In a non-limiting example, it is envisaged in context of the present invention that the identity, amount and/or ratio of the large OPA1 isoforms as defined herein, namely OPA1-L1 and OPA1-L2, can be determined via specific detection of any amino acid stretch of the large OPA1 isoforms lying in N-terminal direction to the amino acid stretches corresponding to the N-terminal amino acids of the “most N-terminal peptide(s)” defined herein of the small OPA1 isoform(s), alternatively and preferred lying in N-terminal direction to the amino acid stretches corresponding to the N-terminal amino acid of the small OPA1 isoforms. In analogy to the above, said amino acid stretch to be detected may be any epitope-bearing portion, or, e.g. any other portion to which a binding molecule as defined herein can bind and said detection may be a detection method as defined and exemplified herein, e.g. a detection method taking advantage of corresponding OPA1 antibodies as defined and exemplified herein, or a detection method taking advantage of other corresponding OPA1 binding molecules as defined herein.

In another particular embodiments of this invention, it is envisaged to distinguish between various types of mitochondrial dysfunction(s)/disease(s). In particular, it is envisaged to differentiate between mitochondrial dysfunction(s)/disease(s) dependent on depletion of mitochondrial DNA and other types of mitochondrial dysfunction(s)/disease(s). Moreover, a quantitative measure of mitochondrial dysfunction and the employment of a corresponding adapted medical intervention is also envisaged. These embodiments of this invention are also based on the findings provided herein and demonstrated in the appended examples (e.g. example 7), e.g. the findings that certain mitochondrial dysfunction(s)/disease(s) correlate with a particular pattern of large and/or small isoforms of the optic atrophy 1 protein (OPA1), e.g. with a certain ratio of OPA1 isoforms (e.g. a certain ratio of OPA1-S5 compared to all OPA1 isoforms (e.g. see example 7 and corresponding table)). The Person skilled in the art is, based on the teaching of the present invention, readily in the position to figure out (further) particular patterns or corresponding ratios of large and/or small isoforms of the optic atrophy 1 protein (OPA1) being specific for certain types of mitochondrial dysfunction(s)/disease(s).

In context of the present invention, it is intended that the identity, amount or ratio of large and/or small isoforms of OPA1 is determined by optical, spectrophotometric and/or densitometric measurements or analysis. Such determination methods are well known in the art. A particular choice of such methods is described in the appended examples. For instance, such methods comprise the SDS-PAGE analysis, Western blots, ELISA, RIA, CLIA, IRMA and/or EIA. These and further methods are known in the art and are, e.g., described in “Cell Biology: Laboratory manual 3rd edition” (2005, J. Celis, editor. Academic Press, New York).

It is also intended that the identity, amount or ratio of large and/or small isoforms of OPA1 is determined by peptide analysis. Again, such peptide analysis methods are well known in the art. For example such peptide analysis methods comprise mass spectrometry methods, like MALDI-MS or LC-MS/MS. The use of these particular mass spectrometry methods are described in the appended examples.

A person skilled in the art is able to figure out further methods for the determination of the identity, amount or ratio of large and/or small isoforms of OPA1. For examples such methods are array-based protein detection and quantification (e.g. as described in Nettikadan, 2006, Mol Cell Proteomics. 5:895-901), immuno-PCR (IPCR; e.g. as described in Adler, 2005, Adv Clin Chem. 39:239-92; Case, 1997, Biochem Soc Trans. 25:374 S; Guo, 2006, Nucleic Acids Res. 34:e62; lmai, 1996, Tanpakushitsu Kakusan Koso. 41:614-7; Ruzicka, 1993, Science. 260:698-9; Sano, 1992, Science. 258:120-2; Zhou, 1993, Nucleic Acids Res. 21:6038-9), fluorescent immuno-PCR (e.g. as described in Niemeyer, 1997, Anal Biochem. 246:140-5), Immuno-detection amplified by T7 RNA polymerase (IDAT; e.g. as described in Zhang, 2001, Proc Natl Acad Sci USA. 98:5497-502), and quantitative mass spectrometry (e.g. as described in Former, 2006, Mol Cell Proteomics, 5, 608-19; Zhou, 2004, Methods Mol Biol, 261, 511-8).

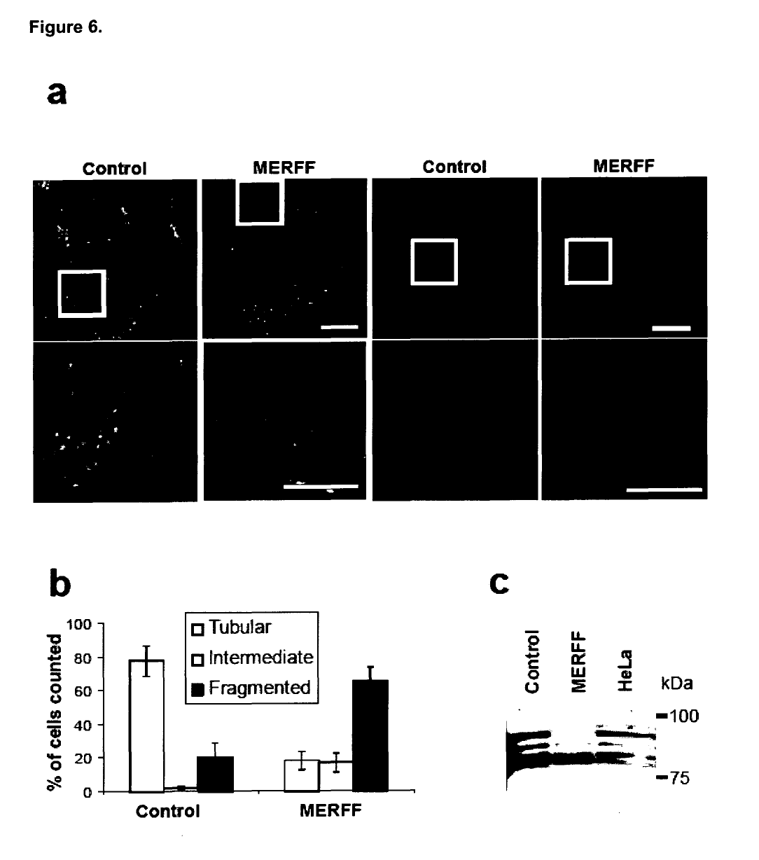

The non-limiting results provided herein document the usefulness of the present invention in particular in context of muscular disorders, like MERRF. As mentioned above, MERRF is myoclonic epilepsy with ragged-red fiber syndrome and MERRF patients suffering from severe myopathy. It was demonstrated herein (e.g. example 5), that in a cybrid cell line derived from a MERRF patient, cells showed highly fragmented mitochondria compared to control cells (FIG. 6ab). The fragmentation status of the mitochondria correlates with the pattern of the five detected OPA1 isoforms in a manner that in the MERRF cells large OPA1 isoforms (particularly OPA1-L1 and OPA1-L2) are reduced and the small OPA1 isoforms (particularly the smallest isoform of OPA1, OPA1-S5) are enhanced.

As detailed herein, one gist of the present invention is based on the finding that a determined reduction of large OPA1 isoforms as described herein (OPA1-L1 and/or -L2) and/or a determined increase of small OPA1 isoforms as described herein (OPA1-S3, -S4 and/or —S5, in particular OPA1-S5) is indicative for the presence of or the susceptibility to a mitochondrial disease/disorder/dysfunction.

Ratios between large and small OPA1 isoforms can be deduced from the appended examples (e.g. example 7 and 8).

The ratio between OPA1-S5 and all OPA1 isoforms may vary between 1% and 25%, preferably between 5% and 15%, for healthy patients and between 10% and 75%, preferably between 15% and 60%, for affected patients. For example, said ratio may be 10% +/−1, 2, 3, 4, or 5% (particularly 9.9%) for healthy patients and 25% +/−1, 2, 3, 4 or 5% (particularly 24.9%) for affected patients. Accordingly, the increase of said ratio of affected patients compared to healthy ones may be in the range of 1 to 12 fold, more preferably in the range of 2 to 8 fold, more preferably in the range of 2 to 6 fold and more preferably in the range of 2 to 3 fold or 4 to 5 fold. For example, said increase may be 2.5 fold or 4.4 fold.

In view of the above, it is envisaged in context of the present invention that, e.g. a ratio between OPA1-S5 and all OPA1 isoforms of more than 10%, preferably of more than 15%, more preferably of more than 20% and most preferably of more than 25% is diagnostic for the “affected patients”. It is to be understood that, in context of the present invention, “affected patients” are patients suffering from a susceptibility for, a predisposition for or the presence of a disorder correlated with mitochondrial dysfunction or mitochondrial disease.

The ratio between OPA1-S4 and all OPA1 isoforms may be 27% +/−1, 2, 3, 4, or 5% (e.g. 27.2%) for healthy patients and 28% +/−1, 2, 3, 4, or 5% (e.g. 28.2%) for affected patients. Accordingly, the increase of said ratio of affected patients compared to healthy ones may be in the range of 1 to 1.5 fold. As mentioned herein above, a decrease of said ratio of affected patients compared to healthy ones, particularly in context of OPA1-S4, is also possible. For example, it is also possible that said ratio does not change between affected patients and healthy ones.

The ratio between OPA1-S3 and all OPA1 isoforms may be 10% +/−1, 2, 3, 4, or 5% (e.g. 9.9%) for healthy patients and 19% +/−1, 2, 3, 4, or 5% (e.g. 19.2%) for affected patients. Accordingly, the increase of said ratio of affected patients compared to healthy ones may be in the range of 1 to 5 fold, more preferably in the range of 1.5 to 3′ fold and more preferably in the range of 1.8 to 2.3 fold. For example, said increase may be particularly 1.9 fold.

The ratio between OPA1-S3, -S4 and -S5 and all OPA1 isoforms may be 47% +/−1, 2, 3, 4, 5 or 10% (e.g. 47.4%) for healthy patients and be 93% +/−1, 2, 3, 4, 5 or 10% (e.g. 93.0%) for affected patients. Accordingly, the increase of said ratio of affected patients compared to healthy ones may be in the range of 1.5 to 3 fold, more preferably in the range of 1.8 to 2.5 fold and more preferably in the range of 1.9 to 2.2 fold. For example, said increase may be particularly 2.0 fold.

The ratio between OPA1-S3 and -S5 and all OPA1 isoforms may be 20% +/−1, 2, 3, 4, or 5% (e.g. 20.2%) for healthy patients and between be 65% +/−1, 2, 3, 4, 5 or 10% (e.g. 64.8%) for affected patients. Accordingly, the increase of said ratio of affected patients compared to healthy ones may be in the range of 2 to 5 fold, more preferably in the range of 2.5 to 4 fold and more preferably in the range of 3 to 3.6 fold. For example, said increase may be particularly 3.2 fold.

In context of values of the ratio of OPA1 isoforms, e.g. the values given above, it is to be understood that within the ranges given for the values, the smaller variations are preferred.

It is intended that the ratios to be determined in context of the present invention may also differ from the ones exemplified above. As already mentioned above, examples that, in a non limiting manner, describe the evaluation of such ratios are given herein below (e.g. Example 7 and 8).

Inter alia, in context of the present invention, the term “ratio” or “density ratio”, inter alia, refers to a comparison of density values of bands corresponding to OPA1 isoforms, as, e.g., derived from an SDS-PAGE/Western-blot. Methods how such density values can be obtained are known in the art and exemplified in the appended non limiting examples.

It is to be understood that not only the comparison of small (OPA1-S3, -S4 and/or -S5) versus large isoforms of OPA1 (OPA1-L1 and/or -L2) or small or large versus other or all OPA1 isoforms derived from an individual patient sample or sample to be tested is of relevance with respect to a certain disorder or disease, but that also a comparison to an healthy control or a corresponding standard is of relevance and can, in accordance with the teachings provided herein, be obtained. This applies, mutatis mutantis, for all methods provided herein.

The person skilled in the art is readily in a position to determine the ratio of individual (or more) OPA1 isoforms as described herein by methods known in the art, like for example densitometric, spectrophotometric, luminescent, autoradiographic or fluorescent quantification methods. Also in this context, methods comprising tests with specific anti-OPA1 isoform antibodies (also specific antibodies against individual OPA1-isoforms as provided herein) are useful. Accordingly, methods, like Western-blot analysis or ELISA/RIA-tests may be employed to determine the OPA1 isoform ratio(s). Corresponding non-limiting examples are illustrated in the appended experimental part.

One gist of this invention is based on the provision of a distinct composition of molecular markers derived from OPA1, i.e. OPA1 isoforms as defined herein, whereby these molecular markers (namely OPA1-L1, -L2, -S3, -S4, -S5) can be measured and analyzed by means and methods provided herein and wherein the composition of the totality of these molecular markers differ between samples obtained from healthy individuals and samples obtained from individuals suffering from or prone to be suffering from a mitochondrial dysfunction or a disorder correlated with mitochondrial dysfunction(s). The composition of molecular markers provided in context of this invention comprises, inter alia, two large isoforms of OPA1 (OPA1-L1, -L2) and three small isoforms of OPA1 (OPA1-S3, -S4, -S5), whereby in particular the ratio between large and small isoforms differs in samples derived from healthy individuals or controls in comparison to samples derived form patients suffering form the mitochondrial dysfunction/disease or patients susceptible to such a disease (“affected patients”). The corresponding novel and inventive finding relates to the fact that in samples derived from individuals who are susceptible for, have predisposition for or have a disorder correlated with mitochondrial dysfunction or mitochondrial disease, the amount of “large isoforms” (OPA1-L1/-L2) is reduced whereas the amount of “small isoforms” (OPA1-S3, -S4, -S5, in particular OPA1-S3 and -S5 and more particular OPA1-S5 as defined herein) is increased. As documented, herein one measure for the determination the susceptibility for, predisposition for or the presence of a disorder correlated with mitochondrial dysfunction or mitochondrial disease is the amount or ratio of the small isoforms versus the large isoforms. Accordingly, the composition of molecular weight markers as provided herein preferably relates to the OPA1 markers in form of OPA1 isoforms (OPA1-L1, -L2, -S3, -S4, -S5 as defined herein), whereby in particular a ratio of more than 10%, preferably of more than 15%, more preferably of more than 20% and most preferably of more than 25% of OPA1-S5 in the total amount of all isoforms (OPA1-L1, -L2, -S3, -S4 and -S5) (as evaluated in e.g. SDS-gels or Western-blots) is diagnostic for the susceptibility for, predisposition for or the presence of a disorder correlated with mitochondrial dysfunction or mitochondrial disease.

Accordingly, as a cut-off and -read-out for the evaluation of normal/healthy versus diseased status or susceptibility, the following values can be taken (as non-limiting examples) when for example the five molecular markers as provided herein are analysed in gels (SDS-gels or Western blots derived from said SDS-gels):

For the ratio between OPA1-S5 and all OPA1 isoforms: More than 10%, preferably more than 15%, more preferably more than 20% and more preferably more than 25%. For the ratio between OPA1-S4 and all OPA1 isoforms: More than 25%, preferably more than 27.5% and more preferably more than 30%. For the ratio between OPA1-S3 and all OPA1 isoforms: More than 10%, preferably more than 15% and more preferably more than 20%. For the ratio between OPA1-S3, -S4 and —S5 and all OPA1 isoforms: More than 50%, preferably more than 70% and more preferably more than 90%. For the ratio between OPA1-S3 and -S5 and all OPA1 isoforms: More than 20%, preferably more than 40% and more preferably more than 60%.

Therefore, the present invention also describes a distinct composition of molecular markers, e.g. on a Western blot or in an SDS-PAGE, which comprise the following bands on said SDS-PAGE or Western blot or proteins corresponding to said bands: An OPA1-L1 band that has an apparent molecular weight of about 97 kD (96.8 kD), an OPA1-L2 band that has an apparent molecular weight of about 92 kD (92.3 kD), an OPA1-S3 band that has an apparent molecular weight of about 88 kD (88.1 kD), an OPA1-S4 band that has an apparent molecular weight of about 84 kD (84.4 kD), and/or an OPA1-S5 band that has an apparent molecular weight of about 81 kD (80.9 kD), and wherein the OPA1-S5 band comprises more than 15% +/−3%, preferably +/−2%, more preferably +/−1% of the density ratio/amount of the all herein defined bands (OPA1-L1, -L2, -S3, -S4 and -S5). In a sample derived from an healthy individual said density ratio/amount is lower than 15% +/−3%, preferably +/−2%, more preferably +/−1%.

In context of this invention, the term “density ratio” or “ratio” means the value of measured amount, e.g. of at least one OPA1 isoform compared to at least one other (or also the same, see above) OPA1 isoform, which can be deduced by standard methods known in the art and which are normally based on optical (also computer-assisted, like image analysis software) measurements. These measurements are also described in the appended examples and comprise scanning of western-blots and densitometric analysis using standard image software, densitometry and spectrometry.

Therefore, it is within the routine skills of the artisan to detect and deduce the individual OPA1 isoforms protein levels (e.g. the optical density ratio), through densitometry, spectrophotometry and the like.

The present invention is further described by reference to the following non-limiting figures and examples.

The Figures show:

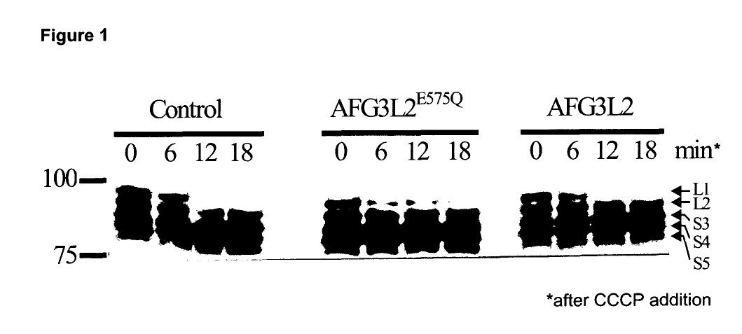

FIG. 1: Overexpression of an inactive variant of AFG3L2 partially inhibits proteolytic processing of OPA1 in HeLa cells.

HeLa cells were co-transfected with a plasmid overexpressing OPA1 splice variant 7 and a plasmid either overexpressing AFG3L2, a proteolytically inactive variant (AFG3L2^{E575Q}), or a GFP variant addressed to mitochondria (Control). The transfected cells were treated with CCCP (20 μM) as indicated. At the indicated time of treatment, the cells were harvested, washed, lysed and subjected to immunoprecipitation with antibodies raised against OPA1. During these last steps o-phenanthroline and/or EDTA were added in order to stop the processing of OPA1. Elution fractions were subjected to western blot analysis using anti-OPA1 antibodies. The different OPA1 isoforms are indicated by arrows and named L1-, L2-, S3-, S4- and S5-OPA1.

FIG. 2. Determination of the apparent molecular weight of OPA1 isoforms by 10% SDS-PAGE.

A, Lane 2, 4, 6: Protein extract from isolated HeLa mitochondria; lane 1: Biorad prestained molecular weight marker; lane 3 PeqLab LMW molecular weight marker; lane 5: Sigma HMW molecular weight marker. Molecular weight of marker proteins (lane 1, 3, 5) is given in kD. OPA1 isoforms with apparent molecular weight larger than 91 kD are defined as large OPA1 isoforms ((l-)OPA1#1 and (l-)OPA1#2). OPA1 isoforms with apparent molecular weight smaller or equal than 91 kD are defined as small OPA1 isoforms ((s-)OPA1#3, (s-)OPA1#4 and (s-)OPA1#5). The dotted white line indicates the 91.0 kD boundary.

B, Molecular weight of all bands representing OPA1 isoforms (lanes 2, 4 and 6 of A) were calculated from logarithmic regression analysis of migration length versus molecular weight for all shown marker bands (lanes 1, 3 and 5 of A).

FIG. 3. Identification of OPA1 isoforms by mass spectrometry.

a, Immunoprecipitation of OPA1 from isolated HeLa mitochondria (15 mg) using anti-OPA1 antibodies. Equal fractions (0.25%) of total mitochondria (T), supernatant (S) and pellet (P) of solubilized mitochondria after clarifying spin, flow through (FT) and 1% of the elution fraction (E) were analyzed by SDS-PAGE and immunoblotting with anti-OPA1 antibodies.

b, Coomassie stained bands of immunoprecipitated OPA1 isoforms. Each band was separately cut out and used for ESI-LC-MS/MS.

c, Alignment of N-termini of the eight OPA1 splice variants. MPP cleavage site N-terminal to F88 (Ishihara, 2006, Embo J 25, 2966-2977) is indicated. Vertical dotted lines indicate the exon boundaries. Grey highlighted areas represent hydrophobic stretches called TM1, TM2a and TM2b (Herlan 2004, J Cell Biol 165, 167-173). Boxes represent peptides found by ESI-LC-MS/MS analysis of any of the different immunoprecipitated OPA1 isoforms. The absence (−) or presence (+) of each peptide in different OPA1 isoforms (L1 to S5) is indicated below the alignment. The confidence of peptide identification is indicated by ‘+++’ (excellent), ‘++’ (very good), or ‘+’ (good) based on the number of identifications in separate ESI-LC-MS/MS runs and on the ion score.

d, OPA1 splice variant 7 (Sp. 7) was expressed in wild type yeast cells (left panel) or was overexpressed in HeLa cells (right panel). Total yeast extracts thereof or mitochondria isolated from HeLa cells (M) were analyzed by western blotting with anti-OPA1 antibodies. Endogenous OPA1 isoforms (endo) in HeLa cell extracts are shown for comparison. Bands are labelled according to apparent corresponding size of OPA1 isoforms in HeLa mitochondria (L1, L2, S3, S4, and S5). Precursor protein (P) and a degradation product (d) are indicated.

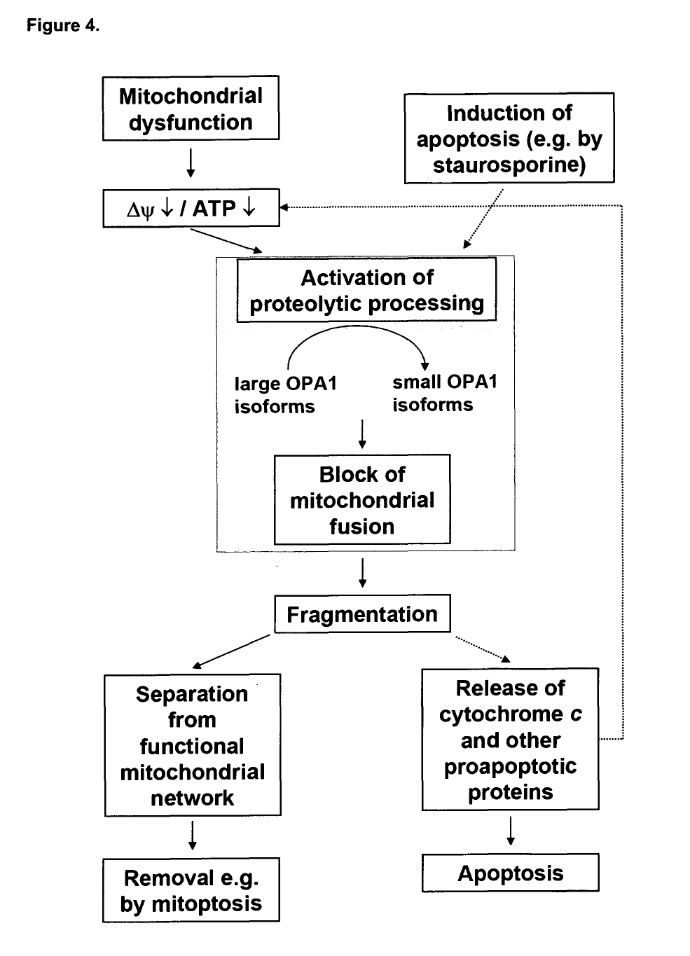

FIG. 4. Model of OPA1-mediated fragmentation of mitochondria in mitochondrial dysfunction and apoptosis.

Mitochondrial dysfunction as observed in a number of human diseases leads to impairment of bioenergetic competence of mitochondria. The reduction of the membrane potential and/or ATP level in mitochondria leads to a proteolytic breakdown of large isoforms of OPA1 to short isoforms. As a consequence, fusion of mitochondria is blocked, and in the presence of ongoing fission, such mitochondria become fragmented. In this way, dysfunctional mitochondria become segregated from the intact network. This may serve two purposes. First, such dysfunctional mitochondria may be removed from cells by autophagy, a process also termed mitoptosis (Skulachev, 2004, Mol Cell Biochem, 256-257, 341-358). Second, a spatial separation as well as a removal would reduce further damage to non-mutated mtDNA caused by ROS produced by a damaged respiratory chain. This mechanism proposes inactivation of OPA1 as a key regulatory step (grey) in counterselecting against damaged mitochondria within cells. The same regulatory step appears to have an early and essential role in apoptosis (dotted arrow lines). Induction of apoptosis, e.g. by staurosporine, activates the proteolytic cleavage of large isoforms of OPA1 to short isoforms. This leads to a block of mitochondrial fusion and fragmentation prior to cytochrome c release. Later in apoptosis cytochrome c and other proapoptotic factors are released from mitochondria and the membrane potential is reduced, leading to further increase of OPA1 processing and mitochondrial fragmentation before cells finally undergo apoptosis.

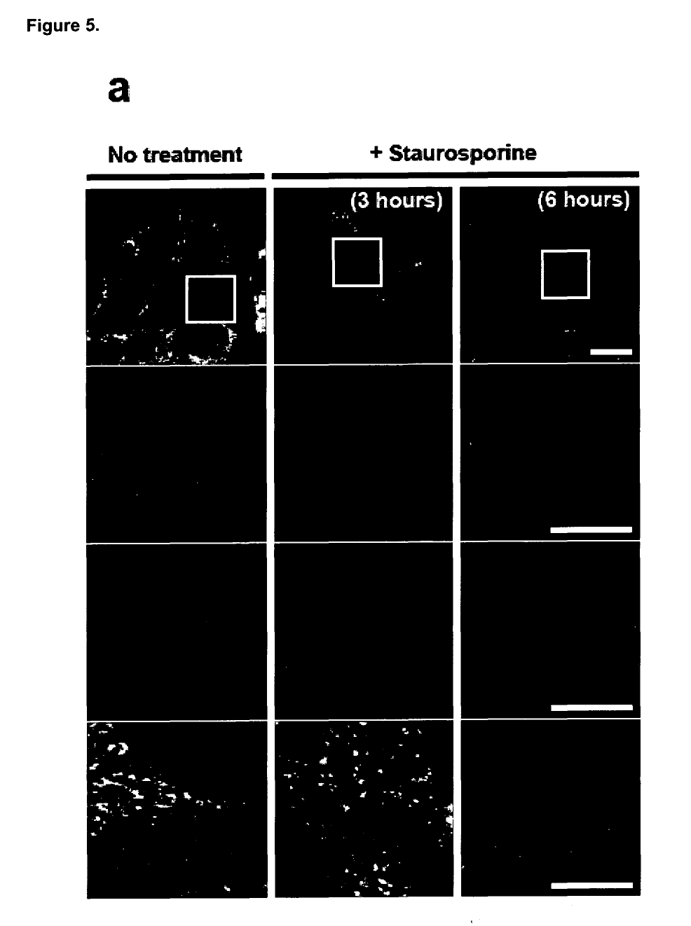

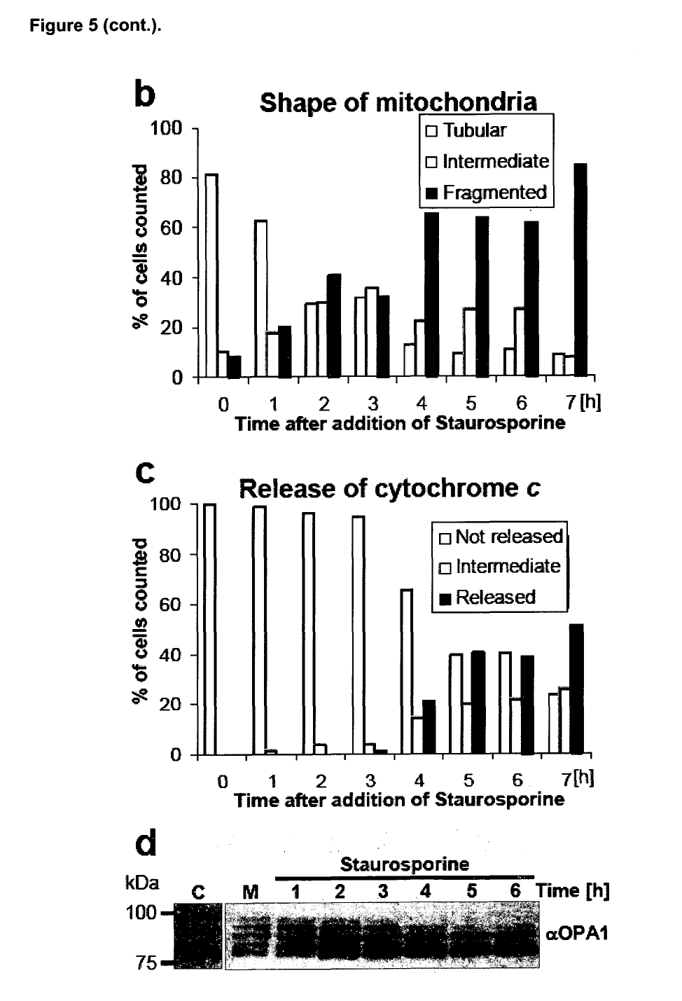

FIG. 5. Mitochondrial fragmentation and alteration of OPA1 protein isoforms precede cytochrome c release in apoptosis.

HeLa cells were treated with 1 μM staurosporine for the indicated time periods, stained with MitoTracker (red) and with cytochrome c antibodies (green).

a, Merged (top row; bottom row) or separately green (2^{nd} row) and red (3^{rd} row) confocal fluorescence images. Top, overview (scale bar 20 μm); bottom, indicated box, (scale bar 10 μm).

b, Cells were classified. Tubular, at least one mitochondrial tubule of 5 μm or more; intermediate, at least one between 0.5 and 5 μm but none more than 5 μm; fragmented, none of more than 0.5 μm in length. A representative experiment out of three is shown.

c, Cells were classified for cytochrome c release by immunostaining. Not released, completely colocalised with MitoTracker; intermediate, partly co-localised with MitoTracker; and released, not co-localised with MitoTracker.

d, OPA1 protein isoforms were determined by western blot analysis of total cell extracts. Total cell extracts (C) and isolated mitochondria (M) from untreated HeLa cells were used as controls.

FIG. 6. MERRF cybrid cells show fragmentation of mitochondria and alterations in OPA1 isoforms.

MERRF and control cybrid cell lines were cultured, stained either with MitoTracker (red) and immunostained against cytochrome c (green) or with MitoTracker (red) and DAPI (blue).

a, Merged confocal fluorescence images are shown (1^{st} column: red and green; 2^{nd} column: red and green; 3^{rd} column: red and blue; 4^{th} column: red and blue. Top, overview (scale bar 20 μm); bottom, indicated box, (scale bar 10 μm).

b, Quantification of mitochondrial morphology in cells: Tubular, at least one mitochondrial tubule of 5 μm or more; intermediate, at least one between 0.5 and 5 μm but none more than 5 μm; fragmented, none of more than 0.5 μm in length. Error bars represent standard deviation of five slides evaluated.

c, OPA1 isoforms by western blot analysis of total cell extracts. In c, HeLa cell extracts are shown for comparison.

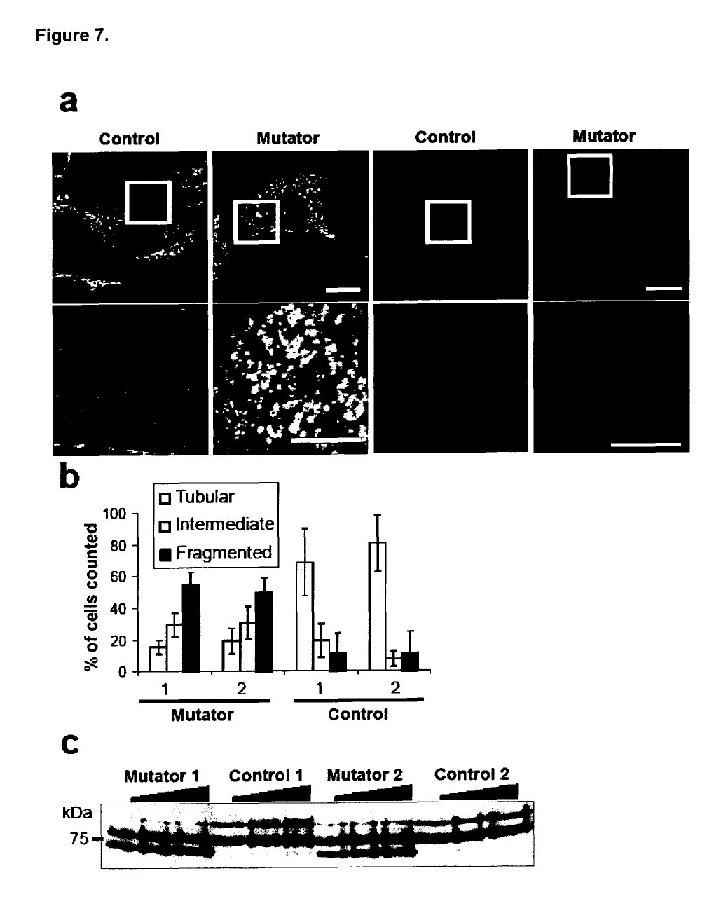

FIG. 7. Mutator mouse embryonic fibroblasts show fragmentation of mitochondria and alterations in OPA1 isoforms.

Immortalized mouse embryonic fibroblasts from two control mice and two mutator mice were cultured, stained either with MitoTracker (red) and immunostained against cytochrome c (green) or with MitoTracker (red) and DAPI (blue).

a, Merged confocal fluorescence images are shown (1^{st} column: red and green; 2^{nd} column: red and green; 3^{rd} column: red and blue; 4^{th} column: red and blue. Top, overview (scale bar 20 μm); bottom, indicated box, (scale bar 10 μm).

b, Quantification of mitochondrial morphology as in FIG. 6b. Error bars represent standard deviation of five slides evaluated.

c, OPA1 isoforms by western blot analysis of total cell extracts.

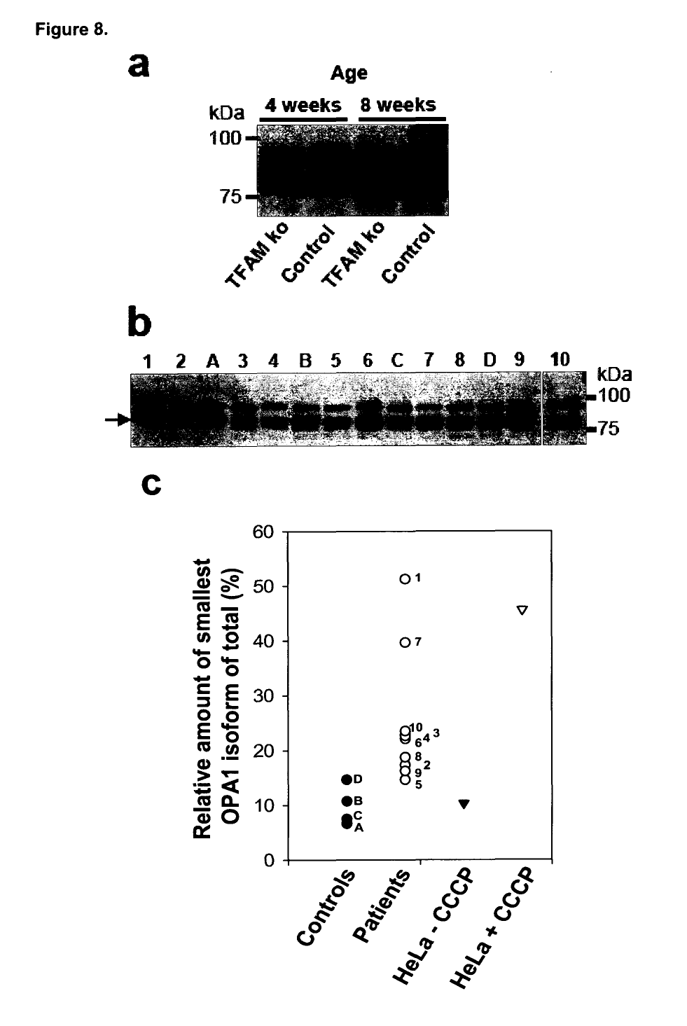

FIG. 8. Patterns of OPA1 isoforms are altered in tissue samples exemplary of mitochondrial dysfunction.

a, Western blot analysis of OPA1 isoforms of heart tissue from mice with a heart-specific knock-out of TFAM (TFAM ko) at 4 or 8 weeks age and of control mice.

b, Homogenates from skeletal muscle biopsies from control individuals (A-D, see Tab. 2) and from patients suffering from respiratory disorders (1-10, see Tab. 2) analyzed by western blotting for OPA1. The smallest form of OPA1, OPA1-S5 (“s-OPA1”), detected is indicated (arrow).

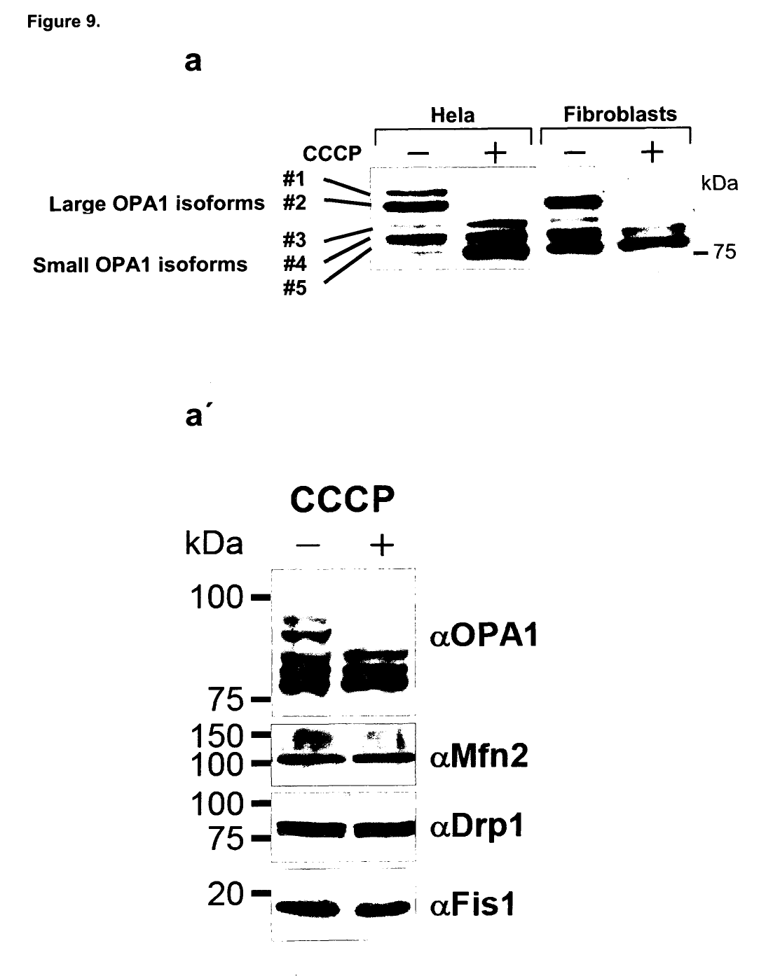

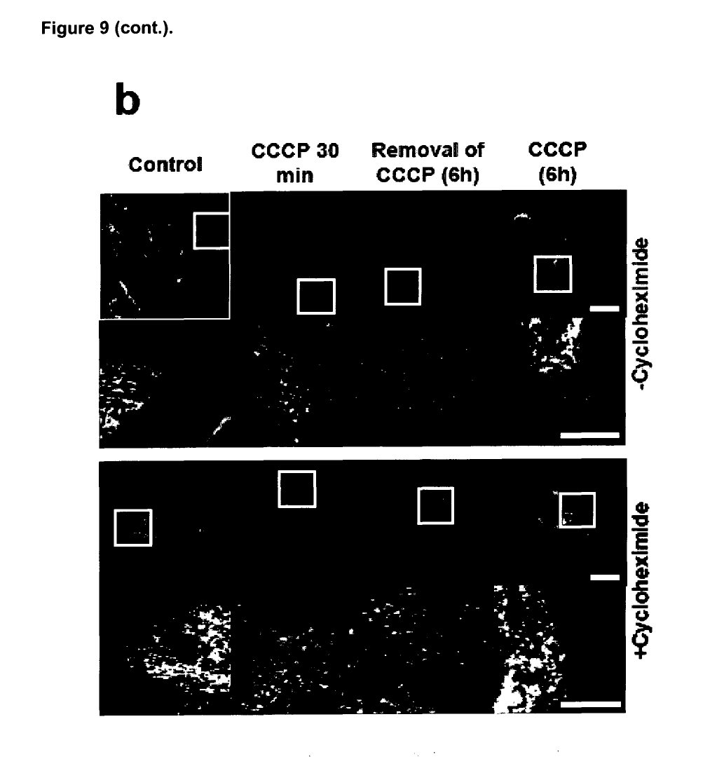

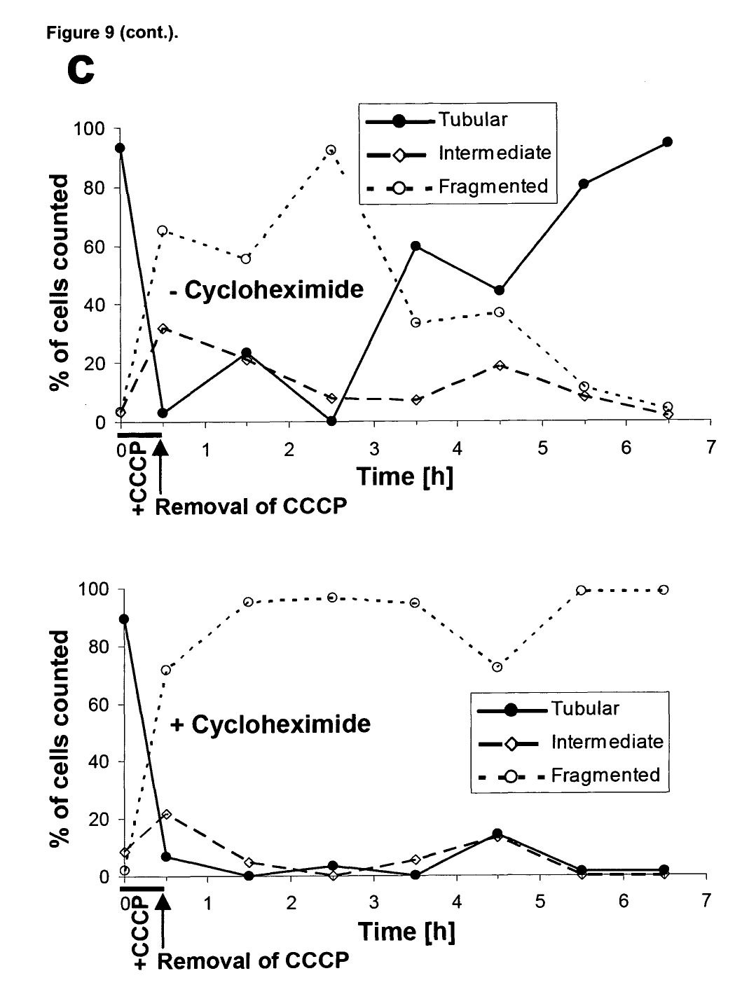

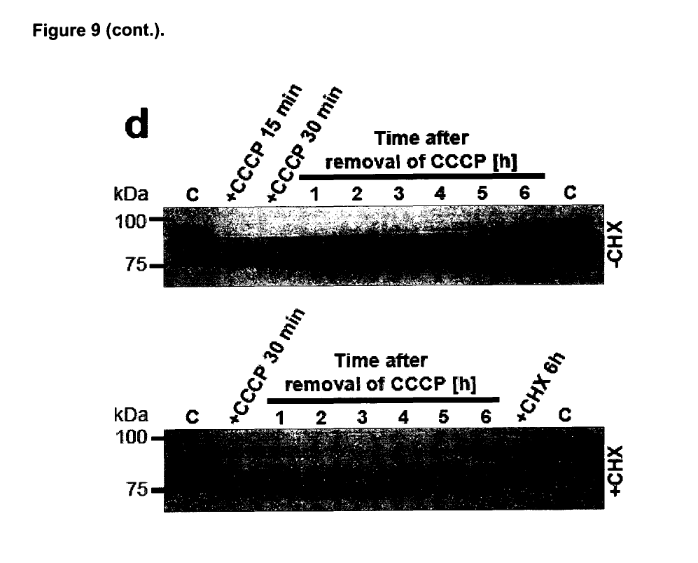

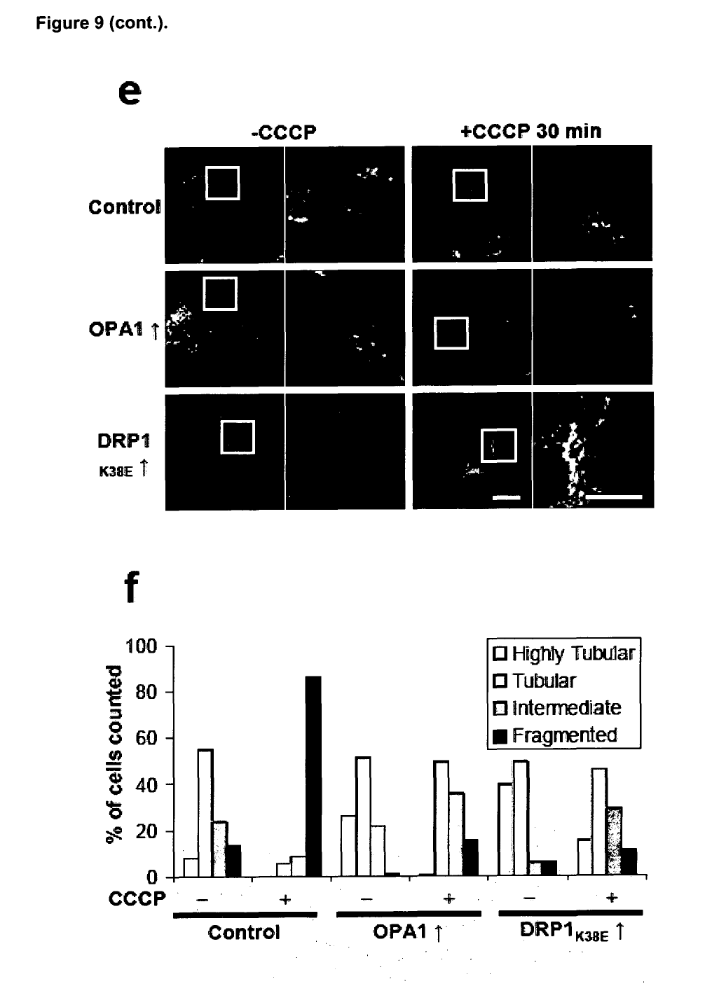

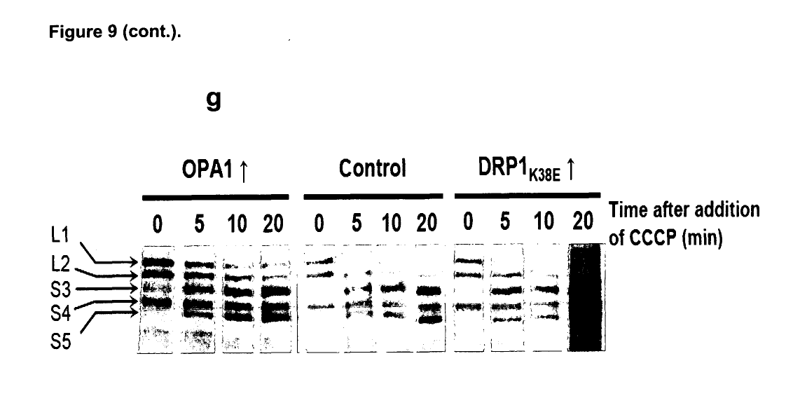

c, Relative amounts of “s-OPA1” (OPA1-S5) of all OPA1 isoforms determined densitometrically from panel b and from HeLa cells treated or not for 6 h with CCCP (see FIG. 9a, two left lanes).

FIG. 9. Mitochondrial fragmentation and OPA1 processing after dissipation of the membrane potential are causally linked.

a, HeLa cells and human fibroblasts were treated with CCCP (20 μM) for 6 h and total cell extracts were subjected to western blotting with OPA1 antibodies. b-d, Cells were treated or not (Control) with CCCP (20 μM) for 30 min, washed, and incubated further with medium lacking CCCP for the indicated time periods or CCCP (20 μM) was left for 6 h. In parallel, these experiments were performed in the presence of cycloheximide (CHX; 175 μg/ml). Cells were stained with MitoTracker (red) and with cytochrome c antibodies (green).

b, Merged confocal fluorescence images are shown. Top, overview (scale bar 20 μm); bottom, indicated box, (scale bar 10 μm).

c, Quantification of mitochondrial morphology as in FIG. 6b.

d, OPA1 isoforms by western blot analysis. Extracts of untreated cells (C) and of cells treated for 6 h with CHX alone (+CHX 6 h) were used as controls.

e, HeLa cells were transfected with a plasmid expressing mitochondrial GFP (control), or co-transfected with this plasmid and a plasmid expressing either a mouse isoform of OPA1 corresponding to spliceform 1 (OPA1↑), or a dominant negative variant of DRP1 (DRP1_{K38E}↑). 36 h after transfection, cells were treated with CCCP for 30 min or not, fixed, and immunostained with GFP (green) and cytochrome c antibodies (red). Merged confocal fluorescence images. Left, overview (scale bar 20 μm); right, indicated box (scale bar 10 μm).

f, Mitochondrial morphology of mitochondrial GFP positive cells was quantified. Cells with at least one highly elongated mitochondrion of more than 10 μm (highly tubular) were quantified in addition to those classes described in FIG. 6b.

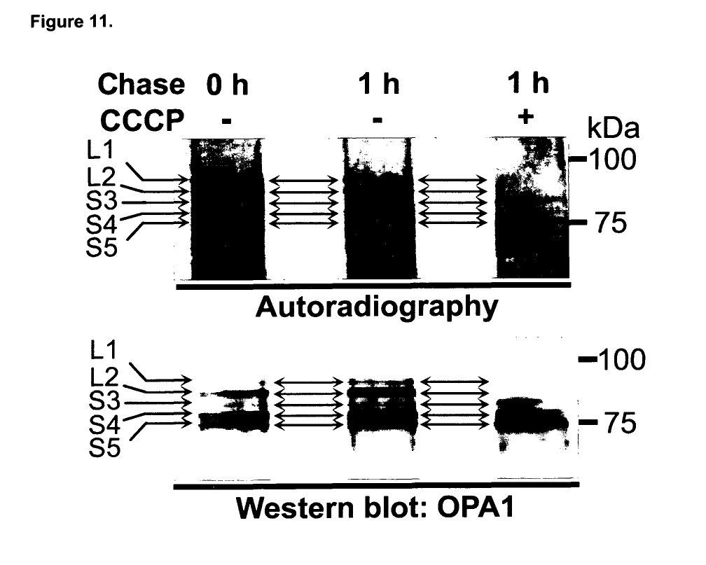

g, Pulse-chase experiment; HeLa cells were transfected as described herein. 24 h after transfection, cells were subjected to radioactive labeling and subsequent CCCP treatment during the chase period as described for FIG. 11. At indicated times after addition of CCCP, cells were washed, harvested, lysed and subjected to immunoprecipitation with antibodies raised against OPA1 in the presence of 5 mM o-phenathroline/10 mM EDTA. OPA1 isoforms in the elution fractions were separated by SDS-PAGE and analyzed by digital autoradiography.

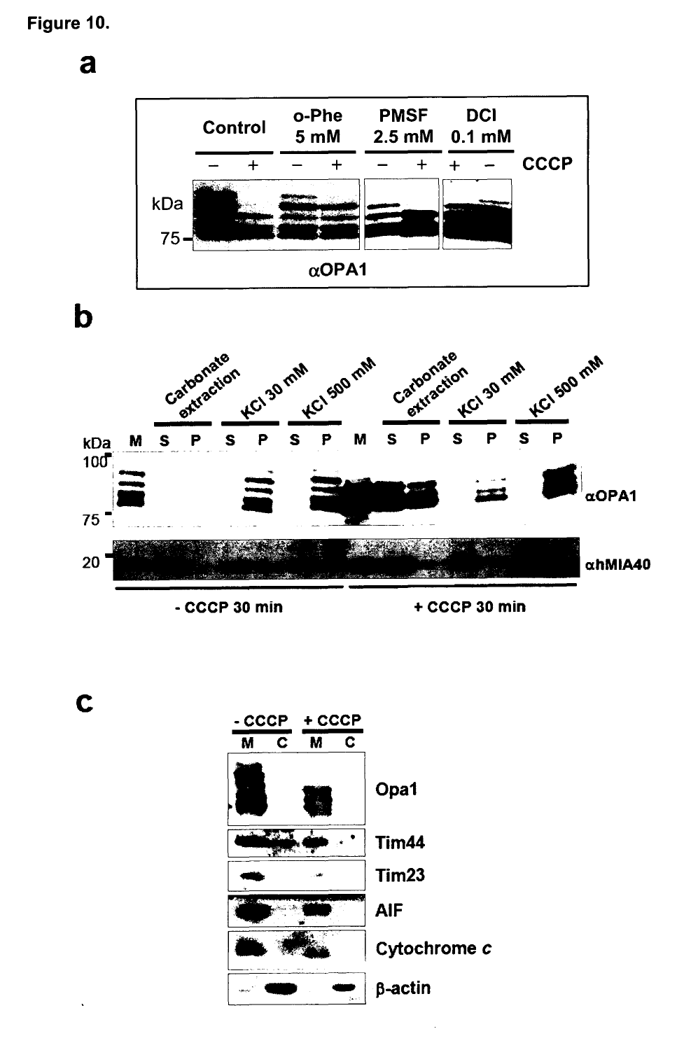

FIG. 10. Processing and membrane association of OPA1 isoforms.

a, The protease inhibitor o-phenanthroline (o-Phe) and partially DCI (3,4-Dichloroisocoumarin), but not phenylmethylsulphonylfluoride (PMSF) block uncoupler induced conversion of larger OPA1 isoforms to smaller isoforms. HeLa cells were preincubated for 10 min with or without the protease inhibitors at the indicated concentration before CCCP (20 μM) was added. Cells were further incubated for 25 min before they were harvested, lysed in loading buffer, loaded on a SDS-PAGE, and immunoblotted with OPA1 antibodies.

a′, HeLa cells were treated or not with CCCP (20 μM) for 30 min and total cell lysates were subjected to western blotting with the indicated antibodies.

b, The larger isoforms of OPA1 behave in salt and carbonate extraction experiments as integral inner membrane proteins, whereas the smaller isoforms behave like peripherally attached proteins. Shorter isoforms remain peripherally attached to mitochondria even after cells were pretreated with CCCP for 30 minutes (right part). Isolated mitochondria from HeLa cells treated with CCCP for 30 minutes (right) or untreated (left) were extracted either with 30 mM or 500 mM KCl after sonication or with 0.1 M sodium carbonate (pH 11). Pellets were recovered by centrifugation (130,000 g, 30 min, 4° C.). 100 μg of mitochondrial proteins were used for extraction and 25% of each pellet (P) and supernatant (S) was analyzed by SDS-PAGE and immunoblotting for the indicated proteins.

c, OPA1 isoforms remain in the mitochondrial fraction independent of pretreatment of cells with CCCP. In addition, neither degradation nor release of other mitochondrial proteins (Tim44, Tim23, AIF, cytochrome c) was observed. As cytosolic marker protein β-actin was used. Hela cells were treated with CCCP as described in b, and subjected to subcellular fractionation. Equal proportions of the mitochondrial fraction (M) and of the cytosolic fraction (C) are analyzed by SDS-PAGE and immunoblotted with indicated antibodies. In total less material was loaded from the CCCP treated derived fractions.

FIG. 11. Pulse-chase experiment.

HeLa cells were subjected to radioactive labeling of newly synthesized proteins. After labeling cells were washed, incubated for the indicated time in the absence of radiolabeled amino acids (chase) either in the presence or the absence of CCCP (20 μM). Cells were lysed at indicated times of chase and subjected to immunoprecipitation with antibodies raised against OPA1. Elution fractions were analyzed by digital autoradiography (top panel) and the same membrane was subjected to western blot analysis using OPA1 antibodies (bottom panel). The different OPA1 isoforms are indicated by arrows and named L1-, L2-, S3-, S4- and S5-OPA1.

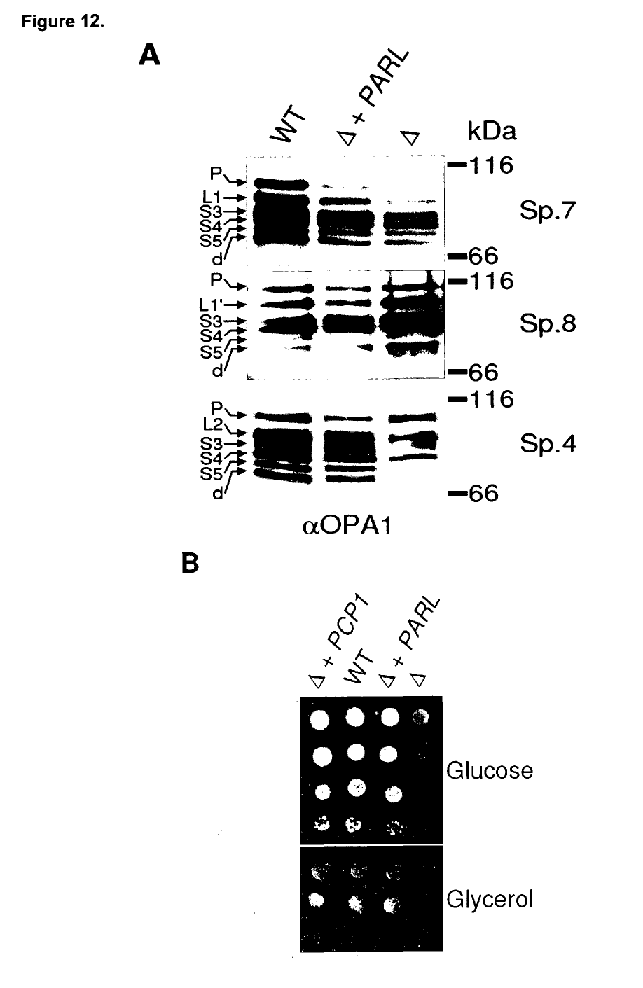

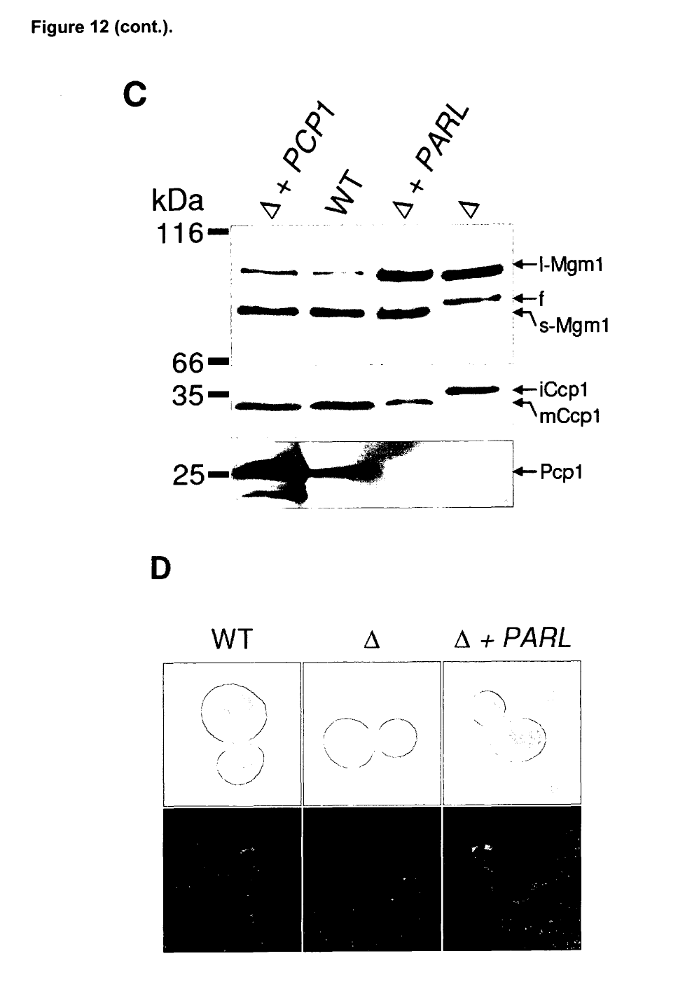

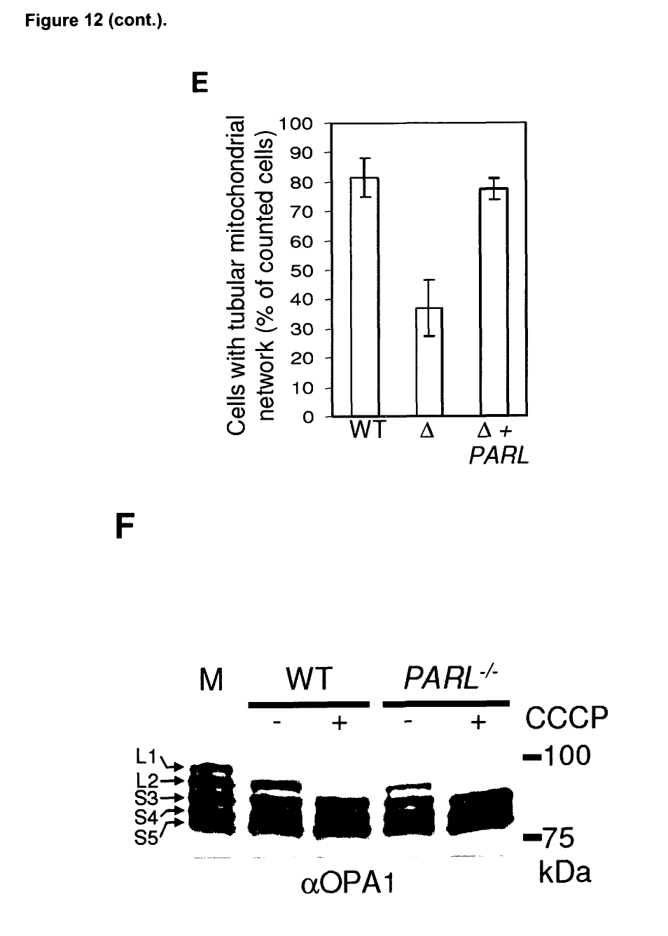

FIG. 12. Processing of OPA1 in yeast does not depend on rhomboid proteases PARL or Pcp1.

(A-E) Functional complementation of Pcp1 by the human mitochondrial rhomboid protease PARL. Wild type (WT) or Δpcp1 (Δ) spores expressing either the human (PARL) or the yeast (PCP1) mitochondrial rhomboid protease were used.

(A) Total cell extracts of the indicated strains expressing OPA1 splice variant (Sp.) 4, 7, or 8 were analyzed by western blotting. For splice variant 8 one band was slightly larger in size than L1 from HeLa mitochondria (not shown) and was therefore labelled L1′. This is consistent with the larger predicted size of the MPP cleaved splice variant 8 as compared to splice variant 7 forming L1.

(B) Growth of indicated strains was tested by drop dilutions on rich media containing indicated carbon sources.

(C) Processing of the two known substrates of Pcp1 was analyzed by western blotting of cell extracts of indicated strains. The bands indicated are the large isoform of Mgm1 (l-Mgm1), the small isoform of Mgm1 (s-Mgm1), a degradation fragment of Mgm1 (f; only in Δpcp1), the mature form of Ccp1 (mCcp1), the intermediate form of Ccp1 (iCcp1; only in Δpcp1). For control anti-Pcp1 immunodecoration is shown. (D and E) Analysis of mitochondrial morphology of the indicated strains expressing a mitochondrial targeted green fluorescent protein (GFP).

(D) Representative images (top, bright field; bottom fluorescence).

(E) Quantification of mitochondrial morphology. Error bars represent the standard deviation (n=3).

(F) Cultured mouse embryonic fibroblasts isolated from Parl^{+/+} (WT) and Parl^{-/-} mice were treated or not with 20 μM carbonyl cyanide 3-chlorophenylhydrazone (CCCP) for 30 minutes. Cell extracts were subjected to western blotting with anti-OPA1 antibodies. HeLa mitochondria (M) were used as a control.

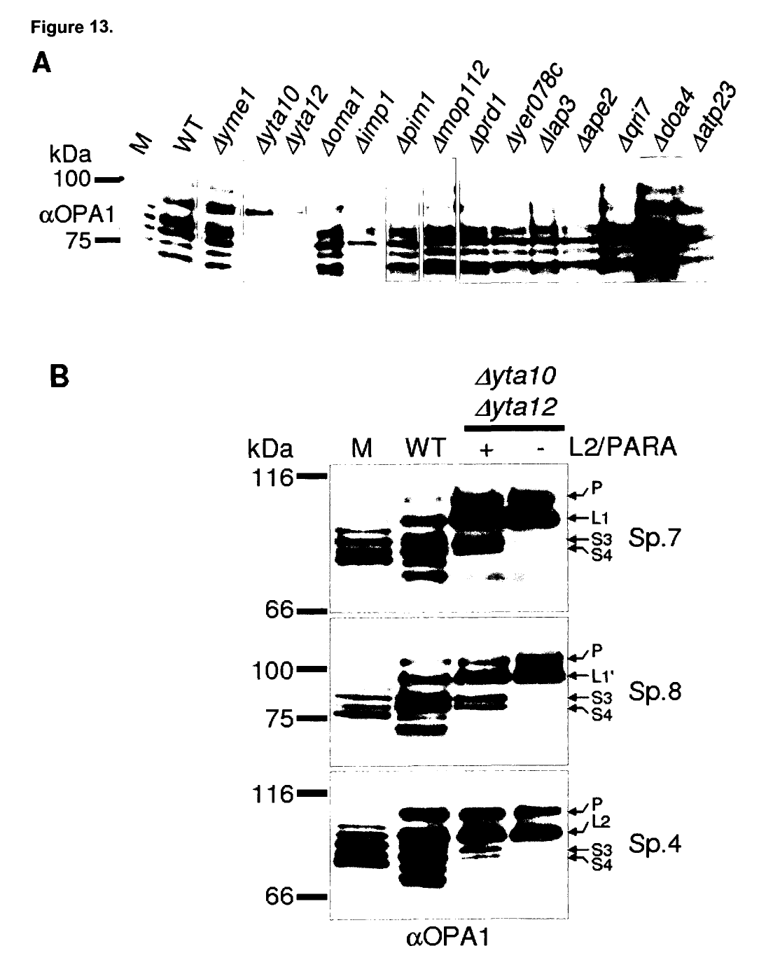

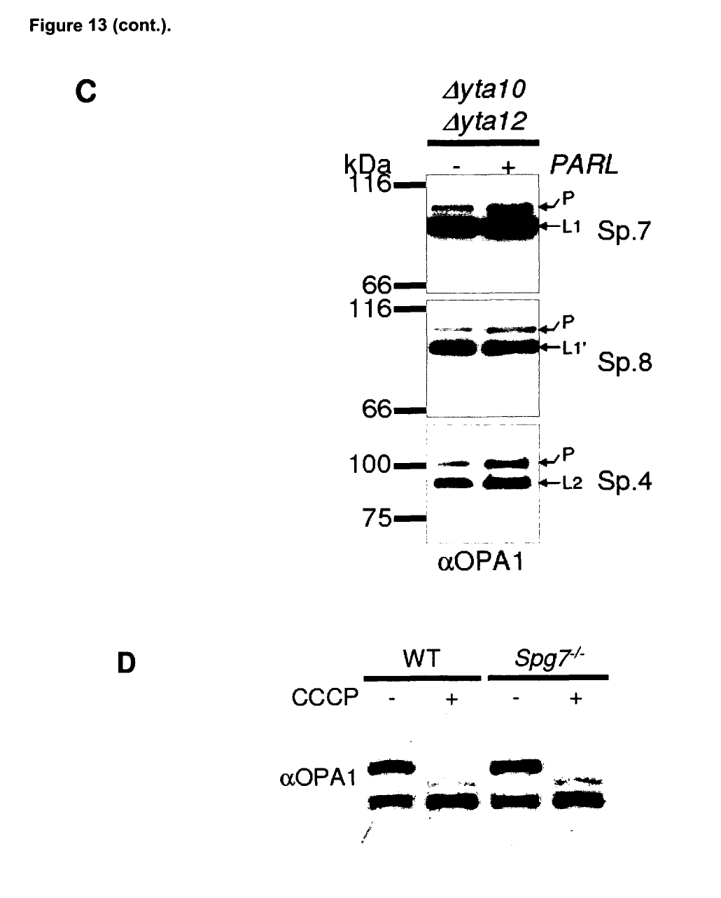

FIG. 13. OPA1 processing depends on the m-AAA protease.

(A) OPA1 splice variant 7 was expressed in yeast strains bearing deletions of putative or known mitochondrial proteases and in wild type (WT) and analyzed by western blotting. HeLa mitochondria (M) are shown for comparison.

(B) Wild type and Δyta10Δyta12 cells complemented (+) or not (−) with their human orthologs AFG3L2 and paraplegin (L2/PARA) expressing OPA1 splice variant (Sp.) 4, 7, or 8. Total cell extracts and HeLa mitochondria (M) were analyzed by western blotting.

(C) OPA1 splice variants 4, 7, and 8 were expressed in Δyta10Δyta12 with (+) and without (−) PARL. Cell lysates were analyzed by western blotting.

(D) Cultured mouse fibroblasts isolated from Spg7^{+/+} (WT) and Spg7^{-/-} mice were treated or not with 20 μM CCCP. Total cell extracts were subjected to western blotting.

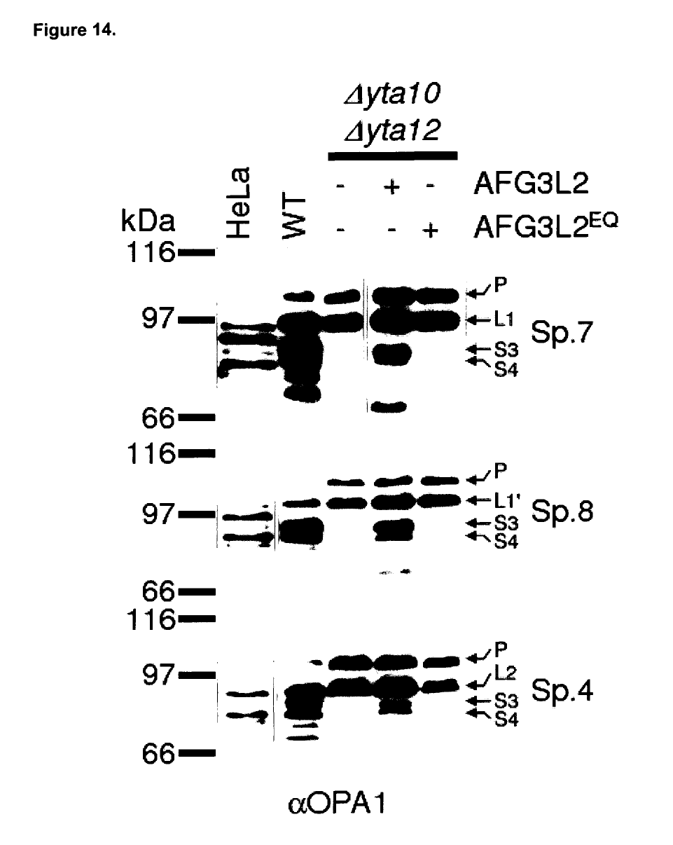

FIG. 14. OPA1 processing by homo-oligomeric human m-AAA protease complexes in the absence of paraplegin.

Human OPA1 splice variants (Sp.) 4, 7, or 8 were expressed in wild type (WT) and in Δyta10Δyta12 cells harbouring the human AFG3L2 or the proteolytically inactive variant AFG3L2^{E575Q} as indicated. Total cell extracts were analyzed by western blotting. HeLa total cell extract was used as reference.

FIG. 15. OPA1 processing depends on the subunit composition of the murine m-AAA protease.

OPA1 was expressed in wild type (WT), Δyta10Δyta12 cells, or Δyta10Δyta12 cells harbouring either murine paraplegin (para), Afg3l1, Afg3l2, or their mutant variants paraplegin^{E575Q} (para^{EQ}), Afg3l1^{E567Q} (Afg3l1^{EQ}) or Afg3l2^{E574Q} (Afg3l2^{EQ}) or combinations of them.

The Examples illustrate the invention.

### EXAMPLE 1

### Material and Methods

### Cell Culture and Reagents:

If not described otherwise, HeLa cells, human fibroblasts, immortalised mouse embryonic fibroblast from control (MEF 13, 14) and mutator mice (MEF 2, 7) (Trifunovic, 2004, Nature 429, 417; Trifunovic, 2005, Proc Natl Acad Sci USA 102, 17993), and cybrid cell lines (pT1, pT3) (Chomyn, 1991, Mol Cell Biol 11, 2236) were grown under standard conditions in Dulbecco modified Eagle's medium (DMEM) containing 4.5 g/l glucose and 2 mM L-glutamine supplemented with 10% fetal bovine serum, 50 U/ml penicillin and 50 μg/ml streptomycin. In addition, immortalized mouse embryonic fibroblasts from control (Parl^{+/+}) and knockout mice (Parl^{-/-}) were used for this study (Cipolat, Cell 126, 163-175, 2006). Moreover, primary dermal fibroblasts isolated from newborn wild type and Spg7^{-/-} mice (Ferreirinha, J Clin Invest 113, 231-242, 2004) were employed. Mammalian cells were cultured in Dulbecco modified Eagle's medium (DMEM) containing 4.5 g/l glucose and 2 mM L-glutamine supplemented with 10% fetal bovine serum, 50 U/ml penicillin and 50 μg/ml streptomycin. Cell culture reagents were obtained from PAA laboratories (Cölbe, Germany), CCCP, o-phenanthroline and cycloheximide were purchased from Sigma (Germany), DCI from Sigma (Germany), and PMSF from Serva (Germany). The OPA1 plasmid (pMSCV-OPA1) was a kind gift of Luca Scorrano (Padova, Italy) (Cipolat, 2004, Proc Natl Acad Sci USA). The DRP1_{K38E} N-terminally fused to CFP (pECFP-C1-DVLP_{K38E}) and mitochondrially targeted GFP (pcDNA3-pOCT:GFP) plasmids were kind gifts of Heidi McBride (Ottawa Heart institute, Canada; Neuspiel, 2005, J Biol Chem, 280, 25060-70; Harder, 2004, Curr Biol, 14, 340-5). Transient transfections of HeLa cells were performed using Metafectene (Biontex Laboratories, Germany). Mitochondria were prepared from HeLa cells by differential centrifugation as described herein following Duvezin-Caubet (loc cit). Transient transfections of HeLa cells were performed using FugeneHD (Roche, Switzerland).

### Yeast Plasmids, Strains and Growth Conditions:

OPA1 splice variants 4, 7 and 8 were amplified from human cDNA and cloned into pYES2 (Invitrogen, USA). All sequences were verified by DNA sequencing. OPA1 splice variant 8 encoded the reported A210V polymorphism (Yao, Mol Vis 12, 649-654, 2006). PARL splice variant 1 was amplified from human cDNA and cloned into pES425#1. Human AFG3L2 together with the ADH1 promoter was subcloned from pRS316-hAFG3L2 (Atorino, J Cell Biol 163, 777-787, 2003) into pRS314 (Sikorski, Genetics 122, 19-27, 1989). PCP1 was amplified from genomic DNA from S. cerevisiae and cloned in pYES2 (Invitrogen, USA). Other plasmids are described in Table 1. For complementation analysis the PCP1/Δpcp1 strain (EUROSCARF, Germany) was transformed with pYES2-PCP1 or pES425-PARL. After sporulation and dissection of tetrads, haploid strains that retained mitochondrial DNA were used for further analysis. Screening of proteases was performed using deletion strains and corresponding wild type cells (BY4742) from BioCat (Open Biosystems, USA) and transformed with pYES2-OPA1 plasmids. Saccharomyces Genome Database (SGD) nomenclature is used throughout. Haploid W303 was used as wild type control elsewhere. All strains used in this study are described in Table 2. Cells were grown under standard conditions (Guthrie, Methods Enzymol 194, 1-270, 1991). Strains expressing OPA1 were cultured using 2% galactose and 0.5% glucose unless indicated differently.

### Tissue Samples:

Heart tissue was obtained from heart-specific TFAM knockout mice as described earlier (Hansson, 2004, Proc Natl Acad Sci USA 101, 3136). Skeletal muscle biopsies were derived from patients diagnosed with respiratory chain disorders or control patients with no such defects (see Tab. 2). Informed consent was given by all patients.

### Antibodies for Immunoblotting:

Anti-OPA1 antibody was affinity purified from a rabbit polyclonal serum raised against the C-terminus of human OPA1 using synthetic peptide: CDLKKVREIQEKLDAFIEALHQEK (SEQ ID No: 29) (Pineda Antikörper-Service, Berlin; BD Biosciences) following Duvezin-Caubet (loc cit). Antibodies against human MIA40 were raised in rabbits using purified MIA40 fused to MBP. Polyclonal rabbit sera against hTim44, and hTim23 were raised in rabbits as described Bauer (1999, J Mol Biol 289(1), 69-82). Antibodies against AIF (goat anti-AIF, D-20:sc-9416, Santa Cruz Biotechnology, USA), rabbit anti-Fis1 sera (IMGENEX), cytochrome c (mouse, clone 7H, 8.2; C12, BD Biosciences), anti-DRP1 (DLP1 clone 8; BD Biosciences) and β-actin (clone AC-15, Sigma, Germany) were used according to the manufacturer's instructions. The anti-Mfn2 serum was a kind gift of Antonio Zorzano (University of Barcelona, Spain). The anti-Mfn2 serum was a kind gift of Antonio Zorzano (University of Barcelona, Spain; Pich Hum Mol Genet. 2005 14(11):1405-1415). Anti-Pcp1 antibodies (Pineda Antikörper-Service, Berlin) were affinity purified from a rabbit polyclonal serum raised against the C-terminus of Pcp1 using the synthetic peptide: CEKQRQRRLQAAGRWF (SEQ ID NO: 36).

### Fluorescence Microscopy:

Live cells were fluorescently labeled with MitoTracker® Red CMXRos (Molecular probes, USA) for mitochondria or with 4′,6-diamidino-2-phenylindole dihydrochloride (DAPI; Molecular probes, USA) for the nucleus, subsequently fixed, and permeabilised. Immunostaining was carried out with mouse anti-cytochrome c monoclonal antibody (clone 6H2.B4; BD Biosciences) and chicken anti-GFP antibody (Ayes Lab Inc., USA). The following secondary antibodies conjugated to fluorescent dyes were used: Alexa Fluor® 488 anti-mouse IgG (H+L) (Molecular probes, USA), Cy3-conjugated anti-mouse IgG and Fluorescein (FITC)-conjugated anti-chicken IgG (Jackson Immunoresearch, USA). Cells were mounted with Prolong® Gold antifade reagent (Molecular probes, USA). All treatments were done according to the manufacturers' instructions. Mitochondrial morphology was analysed by confocal microscopy using a Zeiss LSM 510 (Carl Zeiss Microscopy, Jena, Germany) equipped with a 63× objective. For all imaging, 512×512 pixel images of single confocal planes were acquired and processed with the Bitplane Imaris 4 software. This technique was particularly employed for FIGS. 5 to 7 and 9 and the corresponding examples.

After transformation with the plasmid pVT100U-mtGFP expressing mitochondrially targeted GFP (Westermann, Yeast 16, 1421-1427, 2000) strains were analyzed by standard fluorescence microscopy on an Axioplan 2 microscope (Carl Zeiss MicroImaging, Inc.) with a NA 1.3 oil immersion objective (100×; model Plan-Neofluar; Carl Zeiss MicroImaging, Inc.) and a CCD camera 1.1.0 (Diagnostic Instruments) at RT using Metaview 3.6a software (Universal Imaging Corp.). Quantification of cells with different morphology phenotypes was performed without knowing the identity of the strain by counting 100 cells each time of minimum three samples of each strain. This technique was particularly employed for FIG. 12 and corresponding examples.

### EXAMPLE 2

### Detection of OPA1 Isoforms by SDS-PAGE

Cell pellets were lysed in Lämmli-buffer 1× and heated for 5 min at 95° C. Samples equivalent to ˜10⁶ cells were loaded on a 10% acrylamid gel (12 cm high×16 cm wide plates separated by 1.5 mm thick spacers). Gels were run for 3.5 hours at constant 30 mA or until the 37 kD band of the BioRad Precision Plus Protein standard reaches the bottom of the gel. The proteins were transferred to a nitrocellulose membrane by semi-dry blotting for 1.5 hours at 200 mA using Tris/glycine buffer. The membrane was incubated in TBS buffer containing 5% non fat milk powder for 30 min. The membrane was then incubated in TBS buffer containing 5% non fat milk powder and affinity purified rabbit OPA1 antibodies (raised against the C-terminal peptide of OPA1: DLKKVREIQEKLDAFIEALHQEK (SEQ ID No: 30)) diluted 1:500 for 1 hour. After three rapid washes to remove the primary antibody in excess, the membrane was further incubated for 1 hour in TBS containing 5% non fat milk powder and secondary antibodies anti rabbit conjugated to HRP (horseradish peroxidase) diluted 1:10,000 (BioRad). The membrane was then washed in TBS, once rapidly, and four more times for 5 min each. ECL reagent (enhanced chemoluminescence reagent) was used to reveal the different OPA1 isoform bands on the membrane. Expositions of 1 to 2 min were usually sufficient to detect all OPA1 forms. The compositions of the buffers and gels to be employed are as follows.

4× Lämmli Buffer 100 ml:
8 g SDS
40 ml Glycerin
40 ml 0.6M Tris pH 6.8
80 mg Bromophenol blue
Adjust the volume to 80 ml with distilled water and then add
β-Mercaptoethanol 20 ml

### SDS-PAGE Running Buffer (Electrophoresis Buffer) 10× for 5I:

50 g SDS
150 g Tris
720 g Glycine
   Add distilled water to reach 5l.

### 10% Separating Gel (10%):

| | | |
|---|---|---|
| Acrylamide 30%/N,N′-Methylene-bis-acrylamide 0.5% | 5.7 | ml |
| 1M Tris pH 8.8 | 6.5 | ml |
| Distilled water | 4.5 | ml |
| TEMED | 10 | μl |
| 10% APS | 100 | μl |
| Total (for one gel) | 17 | ml |

### Stacking Gel:

| | | |
|---|---|---|
| Acrylamide 30%/N,N′-Methylene-bis-acrylamide 0.5% | 17 | ml |
| 1M Tris-HCl pH 8.8 | 6 | ml |
| 10% SDS | 1 | ml |
| Distilled water | 76 | ml |
| Total (for 20 gels) | 100 | ml |

### Store at 4° C. Take 5 ml for one gel.

| | | |
|---|---|---|
| Add | 25 μl | 10% APS |
| | 5 μl | TEMED |

### 1× Blotting Buffer:

| | | |
|---|---|---|
| 20 mM Tris | 4.84 | g |
| 150 mM Glycine | 22.52 | g |
| 20% Methanol | 400 | ml |
| 0.08% SDS | 1.6 | g |
| Total | 2 | l (adjusted with distilled water) |

### 10×TBS:

| | | |
|---|---|---|
| 100 mM Tris | 60.57 | g |
| 9% NaCl | 450 | g |
| Total | 5 | l (adjusted with distilled water) |

Adjusted to pH 7.4 with HCl.

### EXAMPLE 3

### Identification of OPA1 Peptides by Mass Spectrometry

Cells were lysed in lysis buffer (0.5% TritonX100, 150 mM NaCl, 10 mM Tris/HCl pH 7.5, 5 mM EDTA, supplemented with complete protease inhibitor cocktail; Roche, Switzerland) and subjected to standard immunoprecipitation. Thereby, OPA1 isoforms from isolated HeLa mitochondria were immunoprecipitated using an antibody raised against the C-terminal peptide of OPA1 covalently coupled to Sulfo-Link sepharose beads (Pierce, USA) or, alternatively, coupled to Protein A Sepharose CL-4B beads (Amersham Biosciences). If not described otherwise, elution was performed with 1× Lämmli-buffer, separated by SDS-PAGE, stained with Coomassie Blue and bands were cut after in-gel digest with trypsin. All bands were clearly identified as derived from OPA1. Cut gel slices from SDS-PAGE were washed 2× with water and 2× with 40 mM ammonium bicarbonate for 30 min each. After 2×5 min treatment with 50% acetonitrile, trypsin (Sequencing Grade Modified, Promega) was added and proteins were digested over-night in 40 mM ammonium bicarbonate at 37° C. while shaking (650 rpm). Peptides were directly analyzed by MALDI-MS or LC-MS/MS. For example, if not described otherwise, peptides were directly analyzed by nano-ESI-LC-MS/MS for which they were separated on a C18 reversed phase column (75 μm i.d.×15 cm, packed with C18 PepMap™, 3 μm, 100 Å by LC Packings) via a linear acetonitrile gradient, MS and MS/MS spectra were recorded on a QSTAR XL mass spectrometer (Applied Biosystems), and analyzed via the Mascot™ Software (Matrix Science) using the NCBI^{nr} Protein Database.

The theoretical molecular weight for a corresponding amino acid sequence was calculated using the program “pi_tool” (http://www.expasy.ch/tools/pi_tool.html; Bjellqvist, Electrophoresis 1993, 14, 1023-1031; Bjellqvist, Electrophoresis 1994, 15, 529-539; Gasteiger, 2005, Protein Identification and Analysis Tools on the ExPASy Server, (In) John M. Walker (ed): The Proteomics Protocols Handbook, Humana Press). Thereby, the option “average” regarding the resolution was used.

In the protein bands corresponding to l-OPA1#1 and #2, as most N-terminal peptides, the following peptides were found, besides various peptides present in all OPA1 spliceforms:

| | |
|---|---|
| YLILGSAVGGGYTAK | (SEQ ID NO: 17) |
| TFDQWK | (SEQ ID NO: 18) |
| DMIPDLSEYK | (SEQ ID NO: 19) |
| WIVPDIVWEIDEYIDFEK | (SEQ ID NO: 20) |
| LAPDFDK | (SEQ ID NO: 21) |
| IVESLSLLK | (SEQ ID NO: 22) |
| ALPNSEDLVK | (SEQ ID NO: 23) |
| DFFTSGSPEETAFR | (SEQ ID NO: 24) |
| (e.g. see FIG. 3c and 3d) | |

Accordingly, in large OPA1 isoforms at least the amino acids from position 102 to the most C-terminal amino acid of all OPA1 spliceforms (SEQ ID No:2, 4, 6, 8, 10, 12, 14, 16) are comprised and the theoretical molecular weights vary between 95.7 and 105.9 kD, depending of the corresponding spliceform to be used for molecular weight calculation (see below).

Based on in silico predictions (prediction of cleavage of the leader sequence by mitochondrial processing peptidase (MPP) using the computer program Mitoprot (Claros, 1996, Eur J Biochem, 241, 779-86)), it was deduced in a first estimation that large OPA1 isoforms already start with amino acid position 95 of all OPA1 spliceforms (SEQ ID No:2, 4, 6, 8, 10, 12, 14, 16), and hence, start with the peptide TRLLKLRYLILGS (SEQ ID NO: 25). According to this finding, the above mentioned theoretical molecular weights are increased by 0.9 kD. However, based on an alternative MPP cleavage site present from amino acid 86 to 88 (Taylor Structure. 2001 Jul. 3; 9(7):615-25), it was deduced that large OPA1 isoforms already start with amino acid position 88 of all OPA1 spliceforms (SEQ ID No:2, 4, 6, 8, 10, 12, 14, 16), and hence, start with the peptide FWPARLATRLLKLRYLILGS (SEQ ID NO: 35). According to this finding, the above mentioned theoretical molecular weights are increased by 1.7 kD instead of 0.9 kD.

None of the above mentioned peptides were detected in protein bands corresponding to small isoforms of OPA1.

Further, in l-OPA1#1 but not in l-OPA1#2 the following peptide was found: AAGQYSTSYAQQK (SEQ ID NO: 27).

From the data provided, it can be deduced that l-OPA1#1 be derived from spliceform 7 and l-OPA1 #2 represents a mixture of two isoforms derived from splice variants 1 and 4.

In the protein band corresponding to s-OPA1#3, as most N-terminal peptides, the peptides IVESLSLLK (SEQ ID No: 22), DFFTSGSPEETAFR (SEQ ID No: 24), and GLLGELILLQQQIQEHEEEAR (SEQ ID NO: 26) were found. Moreover and inter alia, the peptides AAGQYSTSYAQQK (SEQ ID NO: 27) and IDQLQEELLHTQLK (SEQ ID NO: 28) were found. It was deduced that s-OPA1#3 is derived from spliceform 7 and that the corresponding most N-terminal amino acid is 172 (SEQ ID No: 4), or close-by more N-terminal to this position. The theoretically predicted molecular weight of the s-OPA1#3 is about 95.9 kD.

In the protein band corresponding to s-OPA1#4, as most N-terminal peptides, the peptides GLLGELILLQQQIQEHEEEAR (SEQ ID NO: 26) and AAGQYSTSYAQQK (SEQ ID NO: 27) were found. Moreover and inter alia, the peptide IDQLQEELLHTQLK (SEQ ID NO: 28) was found. It was deduced that s-OPA1#4 could derive from spliceforms 4, 6, 7, and/or 8 and that the corresponding most N-terminal amino acid is, dependent on the amino acid sequence of the mentioned spliceforms, at position 173 (SEQ ID No: 10), 191 (SEQ ID No: 6), 209 (SEQ ID No: 4) and 227 (SEQ ID No: 2), respectively. The theoretically predicted molecular weight of the s-OPA1#3 is about 91.8 kD.

In the protein band corresponding to s-OPA1#5, as most N-terminal peptide, the peptide IDQLQEELLHTQLK (SEQ ID NO: 28) was found. It was deduced that s-OPA1#5 could derive from one or more of spliceforms 1 to 8 and that the corresponding most N-terminal amino acid is, dependent on the amino acid sequence of the mentioned spliceforms, at position 215 (SEQ ID No: 16), 179 (SEQ ID No: 14), 197 (SEQ ID No: 12), 216 (SEQ ID No: 10), 233 (SEQ ID No: 8), 234 (SEQ ID No: 6), 252 (SEQ ID No: 4) and 270 (SEQ ID No: 2), respectively.

The theoretically predicted molecular weight of the s-OPA1#5 is about 86.8 kD.

Moreover, in none of the small OPA1 isoforms, the peptide LAPDFDK (SEQ ID NO: 21) was found. Accordingly, is can be deduced that the N-terminal amino acid of each s-OPA1 lies within or shortly C-terminal to this sequence. In either case, the N-terminal amino acid of each small OPA1 lies N-terminal to the above described found most N-terminal peptides of each small OPA1 (see above).

Based on the results from the refinded analyses with respect to the OPA1 peptides determined by the mass spectrometry experiments, for each OPA1 isoform the following theoretically calculated molecular weights and the apparent starting position in the corresponding spliceform of OPA1 (see also the corresponding SEQ ID No) were deduced:

Isoform (amino acid position start/MW in kD)

| Spliceform | I-OPA1#1/#2 | s-OPA1#3 | s-OPA1#4 | s-OPA1#5 |
|---|---|---|---|---|
| 8 | 88/107.6 | x | 227/91.8 | 270/86.8 |
| 7 | 88/105.8 | 172/95.9 | 209/91.8 | 252/86.8 |
| 6 | 88/103.5 | x | 191/91.8 | 234/86.8 |
| 5 | 88/103.4 | x | x | 233/86.8 |
| 4 | 88/101.7 | x | 173/91.8 | 216/86.8 |
| 3 | 88/99.3 | x | x | 197/86.8 |
| 2 | 88/97.4 | x | x | 179/86.8 |
| 1 | 88/101.5 | x | x | 215/86.8 |

To obtain an averaged apparent molecular weight value for either l-OPA1#1 or l-OPA1#2, the four smallest and the four largest molecular weight values of the second column of the above table (“l-OPA1#1/#2”) where averaged. Accordingly, for l-OPA1#1 an averaged molecular weight value of 105.1 kD and for l-OPA1#2 an averaged molecular weight value of 100.0 kD was derived.

Alternatively, based the herein provided evidence that l-OPA1#1 is derived from spliceform 7 and that l-OPA1#2 is derived from spliceform 1 or spliceform 4, for l-OPA1#1 a theoretic molecular weight value of 105.8 kD and for l-OPA1#2 a theoretic molecular weight value of 101.5 kD or 101.7 kD, respectively, was calculated.

At least five distinct OPA1 isoforms are present in HeLa cells, the two high molecular weight OPA1 isoforms L1 and L2 and three isoforms of lower molecular mass S3, S4, and S5 (Duvezin-Caubet loc cit). To examine to which extent the large and/or small OPA1 isoforms are generated by alternative splicing or limited proteolysis, mitochondria from HeLa cells were isolated and the different OPA1 species were purified by immunoprecipitation (FIG. 3ab). The antibodies used were directed against a C-terminal peptide of OPA1 present in all OPA1 isoforms. The various species were resolved by SDS-PAGE and analyzed by LC-MS/MS spectrometry (FIG. 3d). The most N-terminal tryptic peptide found in L1 and L2 was located a few amino acid residues C-terminal to the cleavage site of the mitochondrial processing peptidase (MPP) between A94 and T95 or, alternatively, N87 and F88 (see above and Ishihara loc cit). Specifically for L1 and L2 a number of peptides were found located C-terminally to the MPP cleavage site (FIG. 3d). These peptides were derived from exon 3 and from alternatively spliced exons 4 and 5b but not from exon 4b. Small OPA1 isoforms (S3 to S5) were lacking increasingly more of these peptides from the N-terminus (FIG. 3d). All S-forms lacked the transmembrane segment TM1. Besides that, numerous peptides corresponding to exon 6-29, which are common to all splice variants, were identified in all isoforms (FIG. 3d and data not shown).

In order to assign the isoforms to individual splice variants the presence or absence of peptides in the purified isoforms that cover or overlap exons affected by alternative splicing were taken into account. For L1 peptides that all could be derived from splice variant 7 but not from a different splice variant alone were obtained (FIG. 3). The calculated molecular weights of each splice variant when cleaved by MPP are approximately as follows: 101.5 kDa (Sp. 1), 97.4 kDa (Sp. 2), 99.3 kDa (Sp. 3), 101.7 kDa (Sp. 4), 103.4 kDa (Sp. 5), 103.5 kDa (Sp. 6), 105.8 kDa (Sp. 7), and 107.6 kDa (Sp. 8). Thus, as L2 contained the same nine N-terminal peptides as L1 but was smaller in size (FIG. 3ab) it is unlikely to be derived from splice variant 7 or 8. Further, the peptide pattern found for L2 was consistent with splice variant 1 except for one peptide (GLLGELILLQQQIQEHEEEAR (SEQ ID NO: 26)) which could theoretically be derived from splice variants 4, 6, 7 or 8 (FIG. 3c). However, only the predicted size of MPP cleaved splice variant 4 is nearly identical to the one of splice variant 1. Therefore, in HeLa cells L2 most likely represents a mixture of two isoforms derived from splice variants 1 and 4 whereas L1 is derived from splice variant 7 (FIG. 3c). These results are consistent with the high level of expression of these variants in HeLa cells (Satoh, Biochem Biophys Res Commun 300, 482-493, 2003) and the number and size of bands obtained when splice variants 1, 4, and 7 are expressed in mammalian cells (Ishihara, Embo loc cit; Olichon, Cell Death Differ, 2007, 14(4):682-92). S3 contains peptides representing splice variant 7 while S4 contains peptides derived from splice variants 4, 6, 7, or 8, and S5 contains only peptides common to all eight splice variants. In conclusion, these results suggest that S-forms are generated by proteolysis of larger OPA1 isoforms possibly derived from different splice variants.

### EXAMPLE 4

### The Role of OPA1 During Apoptosis

In a first approach to study the molecular mechanism of fragmentation of mitochondria in higher organisms, the role of fusion-promoting OPA1 during apoptosis was investigated. The levels of OPA1 isoforms in HeLa cells after induction of apoptosis were determined. Treatment of cells with staurosporine resulted in rapid fragmentation of mitochondria within 2-3 h (FIG. 5ab). This coincided with the disappearance of the two largest OPA1 isoforms and a concomitant increase of small isoforms of OPA1 (FIG. 5d). Release of cytochrome c occurred at markedly later time; only after 5-6 h had more than 50% of cells released cytochrome c (FIG. 5c). Thus, fragmentation during apoptosis occurs earlier than release of cytochrome c and concomitantly with disappearance of larger OPA1 isoforms.

### EXAMPLE 5

### Determination of OPA1 Isoforms in MERRF Cells

In a further approach to study the molecular mechanism of fragmentation of mitochondria in human diseases, the role of fusion-promoting OPA1 in a cybrid cell line derived from a MERRF patient suffering from severe myopathy (Chomyn, 1991, Mol Cell Biol 11, 2236) was investigated. The mtDNA in the cell line contained the A8344G mutation in the tRNA^{Lys} in a nearly homoplasmic (˜98%) manner leading to a substantial impairment of mitochondrial function (Chomyn, 1991, Mol Cell Biol 11, 2236). The cells from the MERRF patient, but not control cells, showed highly fragmented mitochondria (FIG. 6ab). The pattern of the five detected OPA1 protein isoforms was altered in the MERRF cells compared to control cells (FIG. 6c). It appears that in the MERRF cells the larger isoforms are reduced compared to, at least, the smallest isoform of OPA1 (OPA1-S5). As OPA1 is required for mitochondrial fusion the observed loss of large OPA1 isoforms may explain the fragmentation of mitochondria in this model system of mitochondrial dysfunction.

### EXAMPLE 6

### Determination of OPA1 Isoforms in Fibroblasts of the ‘Mutator Mouse’

To further substantiate the findings of Example 5, immortalized mouse embryonic fibroblasts derived from the so-called ‘mutator mouse’ (Trifunovic, 2004, Nature 429, 417; Trifunovic, 2005, Proc Natl Acad Sci USA 102, 17993) were analyzed. This mouse was generated by a homozygous knock-in of a variant of mtDNA polymerase γ resulting in a phenotype of premature aging. The variant enzyme has much reduced proofreading activity and mtDNA accumulates random point mutations at a 3-5 fold higher rate than normal leading to severe mitochondrial dysfunction. Mitochondria were extensively fragmented in mutator but not in control cell lines (FIG. 7ab). The levels of the larger OPA1 isoforms were strongly reduced whereas the levels of the smaller forms were increased in the mutant cells (FIG. 7c). These findings suggest that mutations in mtDNA are causative to changes in OPA1 isoform levels and mitochondrial fragmentation.

### EXAMPLE 7

### Determination of OPA1 Isoforms in Heart Tissue of TFAM Knockout Mice

TFAM is an essential mitochondrial transcription factor also required for mtDNA maintenance. A heart-specific knockout of TFAM in mice led to a severe depletion of mtDNA in the heart, resulting in cardiomyopathy and altered mitochondrial morphology (Hansson, 2004, Proc Natl Acad Sci USA 101, 3136). The pattern of OPA1 isoforms in heart tissue of these mice was changed as compared to controls; the abundance of large OPA1 isoforms was reduced whereas that of small isoforms was increased (FIG. 8a). This was even more pronounced at eight weeks compared to four weeks of age (FIG. 8a), consistent with the progression of the cardiomyopathy in those mice (Hansson, 2004, Proc Natl Acad Sci USA 101, 3136). This shows that mitochondrial dysfunction resulting from mtDNA depletion is linked to the reduction of large OPA1 isoforms in the affected tissue. Moreover, the alterations of mitochondrial morphology observed earlier by electron microscopy (Hansson, 2004, Proc Natl Acad Sci USA 101, 3136) are likely due to the shift in the levels of OPA1 isoforms. In addition, it was observed whether alterations in the OPA1 isoform pattern are detectable in patients diagnosed with respiratory chain defects (Tab. 2). Patients 1 to 10 included in this study were previously diagnosed with respiratory chain disorders. All patients suffered from mitochondrial encephalomyopathies or isolated myopathies on the basis of clinical, biochemical, morphological and, in some cases, genetic findings. Skeletal muscle biopsies from patients A to D representing non-mitochondrial disorders served as controls. The activities of the respiratory chain complexes of all control patients were within the normal range. Measurements of rotenone sensitive NADH-ubiquinone oxidoreductase (complex I), succinate-cytochrome c oxidoreductase (complexes II and III) and cytochrome c oxidase (complex IV) were determined spectrophotometrically in skeletal muscle homogenates according to Fischer, 1986, Eur J Pediatr, 144, 441-444, after informed consent was given by the patient. Activities were expressed as units per gram of non-collagenous protein and related to the mitochondrial marker enzyme citrate synthase. A mitochondrial DNA depletion syndrome was diagnosed in patients 1, 4 and 10. In patient 8, a homozygous mutation (G1541A) was identified in the SCO2 gene and in patient 7, mtDNA analysis led to the identification of a heteroplasmic A3243G mutation in the mitochondrial tRNA-Leu_{(UUR)} gene, the MELAS mutation. Homogenates from skeletal muscle biopsies were analysed by western blotting for OPA1 pattern (FIG. 8b). The relative amount of the smallest detected form of OPA1 (“s-Opa1”, which is isoform s-OPA1#5) of all OPA1 isoforms was analyzed densitometrically from the immunoblot (FIG. 8bc).

Indeed, an increase of small OPA1 isoforms was observed in skeletal muscle from these patients but not from controls (FIG. 8b). The ratios of the levels of the smallest OPA1 isoform (“s-Opa1”, which is isoform s-OPA1#5) to the total of all OPA1 isoforms exhibited a much broader variation in patients compared to controls. The patients with mitochondrial DNA depletion syndromes or harboring a MELAS mutation were among those with the highest ratios (FIG. 8c, Table below; patients 1 and 7). In none of the controls, such a shift was observed. This demonstrates that OPA1 isoforms are altered in patients with mitochondrial disorders, in particular those with depletion or mutation of the mtDNA.

| | | | | | % s-OPA1 of |
|---|---|---|---|---|---|
| Samples | Respiratory chain enzyme activities¹ | | | Genetics | total² |
| A | normal | | | — | 6.7 |
| C | normal | | | — | 7.5 |
| B | normal | | | — | 10.7 |
| D | normal | | | — | 14.7 |
| 5 | I: 10% | | | unknown | 14.7 |
| 9 | I: 80% | II/III: 40%; | IV: 20% | unknown | 16.3 |
| 2 | | | IV: 20% | unknown | 17.3 |
| 8 | I: 70% | II/III: 60% | IV: 10% | A1541G mutation | 18.7 |
| | | | | in SCO2 | |
| 6 | I: 30% | | | unknown | 22.1 |
| 4 | I: 30% | | IV: 30% | mtDNA depletion | 22.7 |
| 3 | I: 30% | II/III: 80% | IV: 30% | unknown | 22.9 |
| 10 | I: 50% | II/III: 50% | IV: 20% | mtDNA depletion | 23.5 |
| 7 | I: 50% | II/III: 50% | IV: 80% | MELAS A3243G mutation | 39.7 |
| | | | | in tRNA-Leu^{(UUR)} | |
| 1 | I: 50% | II/III: 50% | IV: 20% | mtDNA depletion | 51.2 |

¹Activities of the respiratory chain complexes I, II/III and IV are expressed in % of the lowest limit of the respective reference range.
²The term “s-OPA1” used in this table denotes the herein definded s-OPA1#5.

### EXAMPLE 8

### Determination of Ratios of OPA1 Isoforms

From the results of the below described uncoupler experiments using HeLa cells (Example 9; FIG. 9a), the ratio of the relative amount of various OPA1 isoforms was determined. For this purpose, total protein extracts were separated by SDS-PAGE and immunoblotted using an antibody raised against the C-terminus of OPA1. The western blot was scanned and densitometrically analysed using standard imaging software. The intensity of each band was determined and the background intensity was subtracted. The results so generated are listed in the following table:

| | OPA1#3 + #5/ | OPA1#3 + #4 + #5/ | OPA1#3/ | OPA1#4/ | OPA1#5/ |
|---|---|---|---|---|---|
| Experiment | all bands % | all bands % | all bands % | all bands % | all bands % |
| HeLa −CCCP | 20.2 | 47.4 | 9.9 | 27.2 | 10.3 |
| HeLa +CCCP | 64.8 | 93.0 | 19.2 | 28.2 | 45.6 |
| fold increase | 3.2 | 2.0 | 1.9 | 1.0 | 4.4 |

### EXAMPLE 9

### Uncoupler Experiments

It was further observed whether dissipation of the membrane potential is sufficient to trigger changes in abundance of OPA1 isoforms and fragmentation of mitochondria. Indeed, treatment of HeLa cells or fibroblasts with the uncoupler CCCP led to a dramatic shift of OPA1 isoforms towards the smaller isoforms (FIG. 8c, 9a). This is apparently due to proteolytic cleavage of the large forms since this process was (at least partially) blocked in the presence of the protease inhibitors o-phenanthroline and DCI (FIG. 10a). Further, the small isoforms produced could be extracted from mitochondria with detergent-free buffer with a higher efficiency than the large forms suggesting that the large forms are integral membrane protein isoforms whereas the small ones are only peripherally attached to the membrane (FIG. 10b). Proteolytic processing occurs within mitochondria as shown by cellular fractionation experiments (FIG. 10c). This is specific for OPA1 since degradation of other mitochondrial proteins is not observed (FIG. 10c). Fragmentation of mitochondria occurred rapidly within 15 to 30 min after addition of CCCP (FIG. 9bc). Processing of OPA1 took place within the same narrow time frame (FIG. 9d). Impairment of fusion of mitochondria may therefore be due to rapid inactivation of OPA1 by proteolysis of large isoforms. Moreover, upon removal of CCCP normal mitochondrial morphology was recovered and this coincided with the reappearance of large isoforms of OPA1 (FIG. 9cd). Mitochondrial fragmentation and OPA1 processing are not accompanied by cytochrome c release in this or in any of the investigated models of mitochondrial dysfunction (FIG. 6a; 7a; 9b; 10c and data not shown). This suggests that mitochondrial fragmentation per se does not result in apoptosis. It was tested whether uncoupler induced fragmentation and reversal of fragmentation require protein synthesis. The protein synthesis inhibitor cycloheximide (CHX) did not interfere with fragmentation and OPA1 processing (FIG. 9cd). This indicates that fragmentation of mitochondria and activation of OPA1 cleavage are independent of protein synthesis. However, recovery of a tubular mitochondrial network as well as the reappearance of large OPA1 isoforms was impaired in the presence of CHX (FIG. 9cd). Therefore, mitochondrial fragmentation and the shift of OPA1 isoforms from large to small are not reversible without ongoing protein synthesis, consistent with the explanation that a proteolytic inactivation of OPA1 causes mitochondrial fragmentation. In order to show the causal and specific role of OPA1 in this process it was investigated whether OPA1 overexpression can block uncoupler-induced fragmentation. Indeed, fragmentation of mitochondria after CCCP treatment was largely prevented upon overexpression of OPA1 (FIG. 9ef). This suggests that OPA1 is directly involved in the fragmentation of mitochondria induced after loss of the mitochondrial membrane potential. A similar effect was observed after the expression of a dominant-negative variant of DRP1 (DRP1_{K38E}) that prevents fission of mitochondria (FIG. 9ef). Thus, the fragmentation that occurs by a block of mitochondrial fusion depends on the normal DRP1-dependent fission pathway.

### EXAMPLE 10

### Pulse-Chase Experiments

The large OPA1 isoforms l-OPA1#1 and l-OPA1#2 are converted to the small OPA1 isoforms s-OPA1#3, s-OPA1#4 and/or s-OPA1#5. These findings were demonstrated by a pulse-chase experiment in which HeLa cells were grown under standard growth conditions in 500 μl DMEM w/o methionine/cysteine for 30 min (starvation), then pulse-labelled by the addition of 50 μCi ³⁵[S]-methionine/cysteine (5 μl) for 2.5 hours. Then cells were washed 3 times with PBS and 1 ml fresh DMEM medium +10% FCS without radioactive methione (chase) was added. Cells were either treated with 20 μM CCCP for 1 hour or not. OPA1 was immunoprecipitated using an antibody raised against the C-terminus of OPA1 and OPA1 was detected by autoradiography and by western-blotting after SDS-PAGE. By both methods a shift from the large isoforms to the short isoforms was detected upon treatment with CCCP (FIG. 11). Accordingly, it can be concluded that l-OPA1 isoforms are actually converted to s-OPA1 isofoms, in particular towards s-OPA1#5.

### EXAMPLE 11

### Determination of the Apparent Molecular Weight of OPA1 Isoforms by 10% SDS-PAGE

Mitochondria were prepared from HeLa cells by standard methods using differential centrifugation. For this purpose, cells were, for example, harvested, washed in phosphate-buffered saline supplemented with 5 mM EDTA, and resuspended in 1 ml of RSB buffer (10 mM HEPES (pH 7.5), 1 mM EDTA, 210 mM mannitol, 70 mM sucrose, supplemented with complete protease inhibitor mixture). Mitochondria were prepared after disruption of HeLa cells by passing 6 times through a 26G needle fitted to a 5 ml syringe applying a method adapted from Arnoult, 2005, J Biol Chem, 280(42), 35742-50. Cell pellets from low speed centrifugations (2,000×g, 10 min, 4° C.) were resuspended in RSB buffer and passaged again through a needle as described. This step was repeated 3 to 4 times. The supernatants from low speed centrifugations were pooled and centrifuged again (13,000×g, 10 min, 4° C.) to obtain a crude mitochondrial pellet and a cytosolic supernatant. Three aliquots of a protein extract (24 μg each) from isolated HeLa mitochondria were separated by 10% SDS-PAGE as described above (see example 2 and FIG. 2). Three different molecular weight markers were loaded next to these (BioRad, Germany, Precision Plus Protein Standard All Blue, #1610373; PeqLab, Germany, Protein MW marker, #27-1010; Sigma, Germany, HMW marker, #M3788). Five OPA1 isoforms with apparent average molecular weights of 96.8 kD, 92.3 kD, 88.1 kD, 84.4 kD, and 80.9 kD were observed (FIG. 2). The molecular weight was determined by logarithmic regressions analysis of the migration length all marker bands with known molecular weights, determining the migration length of all OPA1 isoforms, and subsequently calculating the apparent molecular weight in kD. The bands larger than 91 kD are defined as large OPA1 isoforms ((-)OPA1#1 and (l-)OPA1#2). OPA1 isoforms with apparent molecular weight smaller or equal than 91 kD are defined as small OPA1 isoforms ((s-)OPA1#3, (s-)OPA1#4 and (s-)OPA1#5).

### EXAMPLE 12

### Proteolytic Processing of OPA1 in Yeast

In order to test whether OPA1 processing can be recapitulated in yeast, human OPA1 splice variant 7 was expressed in yeast. In total, five major and a sixth minor OPA1 band were observed (FIG. 3d). Four bands were identical in size to OPA1 isoforms L1, S3, S4, and S5 present in HeLa mitochondria. Moreover, an additional degradation product (d) and a large sized species of very low abundance most likely corresponding to the precursor protein (p) was observed. In agreement with the mass spectrometric analysis of OPA1 isoforms in HeLa cells (FIG. 3c), L2 did not accumulate in yeast cells expressing the splice variant 7 of OPA1 (FIG. 3d). To further corroborate the results, the OPA1 isoforms were purified by immunoprecipitation from total yeast extracts and analyzed the different processing products by LC-MS/MS. The peptide patterns obtained were consistent with those obtained for the corresponding isoforms in HeLa cells (data not shown). In particular, the same most N-terminal peptide was found in L1-like isoforms from yeast and HeLa cells, while the smaller S-like forms lacked the same N-terminal peptides as the corresponding bands in HeLa cells. These data indicate that OPA1 is processed in a similar manner in yeast and human mitochondria.

To further corroborate the results, the OPA1 isoforms were purified by immunoprecipitation from total yeast extracts and the different processing products were analyzed by LC-MS/MS. The peptide, patterns obtained were consistent with those obtained for the corresponding isoforms in HeLa cells (data not shown). In particular, the same most N-terminal peptide was found in L1-like isoforms from yeast and HeLa cells, while the smaller S-like forms lacked the same N-terminal peptides as the corresponding bands in HeLa cells. Taken together, these data indicate that OPA1 is processed in a similar manner in yeast and human mitochondria.

### EXAMPLE 13

### Yeast and Mammalian Rhomboid Proteases are not Required for OPA1 Processing

To investigate whether rhomboid proteases mediate processing of OPA1 in yeast, OPA1 cleavage in Δpcp1 cells lacking yeast rhomboid was examined and human PARL was also expressed in these cells. A heterozygous PCP1/Δpcp1 strain expressing PARL from a plasmid was sporulated and individual spores were analyzed further. The same pattern of OPA1 isoforms upon expression of OPA1 splice variant 7 was observed, irrespective of the presence or absence of Pcp1 or PARL (FIG. 12A). This is a puzzling result as Pcp1 cleaves Mgm1, the yeast homologue of OPA1, and human PARL was shown to restore proteolytic processing of Mgm1 in Δpcp1 cells (McQuibban, Nature 423, 537-541, 2003). Therefore, it was investigated to which extent PARL is taking over the function of Pcp1, and the maintenance of the mitochondrial genome and normal mitochondrial morphology in Δpcp1 cells containing PARL was examined. Expression of either PARL or of Pcp1 rescued the growth defects of the Δpcp1 mutant on fermentable and on non-fermentable carbon sources demonstrating stabilization of mitochondrial DNA (FIG. 12B). The known Pcp1 substrates Mgm1 and Ccp1 (Esser 2002 J Mol Biol 323, 835-843; Herlan 2003 J Biol Chem 278, 27781-27788; Sesaki 2003 Biochem Biophys Res Commun 308, 276-283) were processed in these cells (FIG. 12C), consistent with an earlier report (McQuibban 2003 Nature 423, 537-541). In addition, PARL generated Mgm1 isoforms in the absence of Pcp1 at a ratio compatible with the maintenance of a normal mitochondrial network (FIG. 12DE). Thus, PARL can substitute for Pcp1 and is enzymatically fully active upon expression in yeast. It was reasoned that possibly only certain OPA1 splice variants could be cleaved by PARL or Pcp1. Therefore, splice variants 4, and 8 of OPA1 were also expressed in yeast cells containing Pcp1 or PARL. However, the presence or absence of either of the rhomboid proteases did not affect the processing of any of these OPA1 splice variants (FIG. 12A). Thus, neither Pcp1 nor PARL affect processing of OPA1 in yeast suggesting that another protease is responsible for the formation of small isoforms of OPA1.

To substantiate these observations the requirement of PARL for OPA1 processing in mammalian cells using murine embryonic fibroblasts derived from a Parl^{-/-} knockout mouse was examined (Cipolat 2006 Cell 126, 163-175). The pattern of OPA1 isoforms was not altered in Parl^{-/-} knockout cells when compared to wild type cells (FIG. 12F). Thus, PARL is not required for processing OPA1 isoforms during growth of fibroblasts. Moreover, PARL was dispensable for uncoupler-induced processing of OPA1 which occurred both in wild type and Parl^{-/-} cells to a similar extent (FIG. 12F).

### EXAMPLE 14

### OPA1 Processing in Yeast Depends on the m-AAA Protease

Series of yeast strains lacking putative or known mitochondrial proteases were screened for the impairment of processing of OPA1 splice variant 7. In agreement with a previous report (Ishihara 2006 Embo J 25, 2966-2977), small isoforms did not accumulate in cells lacking the yeast m-AAA protease subunits Yta10 or Yta12 (FIG. 13A). Formation of L1 was not affected consistent with the results that L1 generated upon import into mitochondria by the mitochondrial processing peptidase MPP. In addition, a band most likely representing the precursor form of OPA1 (p) was observed. Similar results were obtained by expressing the OPA1 variants 4 and 8 in these mutants (data not shown). In addition to the above, the role of the human m-AAA protease for OPA1 processing in yeast was examined. A complex of human AFG3L2 and paraplegin has been demonstrated to functionally replace the yeast m-AAA protease (Atorino loc cit). Therefore, OPA1 splice variant 7 was expressed in Δyta10Δyta12 cells harboring human AFG3L2 and paraplegin (FIG. 13B). In addition to L1 and the precursor form, S3 and S4 accumulated in the presence of these m-AAA protease subunits. Notably, in contrast to wild type cells, the OPA1 isoform S5 was not generated in Δyta10Δyta12 cells complemented with human AFG3L2 and paraplegin (FIG. 13B). Similar sized bands (L1, S3, and S4) accumulated in HeLa cells upon expression of splice variant 7 of rat OPA1 (Ishihara loc cit). A similar dependency on human AFG3L2 and paraplegin for the processing of OPA1 splice variants 4 and 8 was also observed (FIG. 13B). The three bands (L2, S3, and S4) observed for splice variant 4 in this yeast mutant corresponded to those described by Olichon (loc cit) when this splice variant was expressed in HeLa cells. Therefore, it was concluded that the human m-AAA protease composed of AFG3L2 and paraplegin can restore OPA1 processing in Δyta10Δyta12 cells. In contrast, overexpression of PARL in m-AAA protease-deficient yeast cells did not lead to the formation of small OPA1 isoforms (FIG. 13C) supporting the conclusion that PARL is not required for OPA1 processing.

### EXAMPLE 15

### Processing of OPA1 Can Occur in Paraplegin-Deficient Spg7^{-/-} Mice

To some extend, the above findings somehow support of a recent report linking the function of paraplegin to OPA1 cleavage in HeLa cells (Ishihara loc cit). However, as only a minor effect of paraplegin downregulation on OPA1 processing was observed in these experiments, the formation of OPA1 isoforms in fibroblasts isolated from paraplegin-deficient Spg7^{-/-} mice was examined (FIG. 13D). No difference was observed in the pattern of OPA1 isoforms under steady-state conditions as well as upon dissipation of the mitochondrial membrane potential in mutant cells as compared to control fibroblasts (FIG. 13D). Furthermore, the steady state concentrations and the pattern of OPA1 isoforms in mitochondria isolated from brain, spinal cord, and liver of Spg7^{-/-} mice were indistinguishable from wild type (data not shown). Thus, processing of OPA1 in mammalian mitochondria can occur independently of paraplegin.

### EXAMPLE 16

### OPA1 Processing by Homo-Oligomeric Human m-AAA Protease Complexes in the Absence of Paraplegin

Recently, a homooligomeric isozyme of the m-AAA protease in mammalian mitochondria was described (Koppen loc cit). To examine whether homo-oligomeric human AFG3L2 complexes can mediate OPA1 processing, splice variant 7 of OPA1 was expressed in Δyta10Δyta12 yeast cells harboring human AFG3L2 or its proteolytically inactive variant AFG3L2^{E595Q}. Processing of OPA1 was restored in Δyta10Δyta12 cells by expressing human AFG3L2 (FIG. 14) and occurred with similar efficiency as observed upon expression of both human paraplegin and AFG3L2 (FIG. 13B). In contrast, expression of AFG3L2^{E575Q} did not promote OPA1 cleavage demonstrating that processing depends on the proteolytic activity of human AFG3L2 (FIG. 14). A similar dependency on human AFG3L2 was also observed for the processing of OPA1 splice variants 4 and 8 (FIG. 14). This demonstrates that homo-oligomeric AFG3L2 complexes recognize and cleave OPA1 in the absence of paraplegin.

### EXAMPLE 17

### OPA1 Processing by Homo- and Hetero-Oligomeric Murine m-AAA Proteases

To obtain further evidence in support of the involvement of specific m-AAA protease isozymes in OPA1 processing, the generation of OPA1 isoforms by murine m-AAA protease complexes differing in their subunit composition was analyzed. In addition to homologs of human AFG3L2 and paraplegin, a further subunit, Afg3l1, is expressed in mice (Kremmidiotis 2001 Genomics 76, 58-65). This leads to an even higher number of possible m-AAA protease complexes with different subunit composition in the inner membrane of murine mitochondria (Koppen loc cit). Both homo-oligomeric Afg3l1 and Afg3l2 complexes as well as hetero-oligomeric assemblies of both proteins with paraplegin were generally shown to be proteolytically active (Koppen loc cit). To assess the activity of these complexes towards OPA1, first, the OPA1 splice variant 7 was expressed in Δyta10Δyta12 yeast cells harbouring murine paraplegin, Afg3l1, or Afg3l2 (FIG. 15). Cleavage of OPA1 upon expression of paraplegin alone was not observed (FIG. 15) consistent with the previous notion that Afg3l1 and Afg3l2 but not paraplegin can form homo-oligomeric, proteolytically active complexes (Koppen loc cit). In contrast, expression of each of the murine subunits, Afg3l2 or Afg3l1, in Δyta10Δyta12 cells promoted OPA1 processing (FIG. 15). Cleavage was abolished when point mutations were introduced in the proteolytic center of Afg3l1 or Afg3l2. Interestingly, expression of Afg3l2 preferentially led to the formation of the S3 isoform, whereas the isoform S4 predominantly accumulated in the presence of Afg3l1 (FIG. 15). Further, Afg3l1 and Afg3l2 complexes mediated processing of OPA1 splice variants 4 and 8 in a similar manner (FIG. 15). These findings demonstrate that OPA1 splice variants 4, 7, and 8 are recognized and cleaved by homo-oligomeric murine Afg3l1 and Afg3l2 complexes in yeast and provide first evidence for different substrate specificities of m-AAA protease complexes composed of different subunits.

Coexpression of paraplegin with either Afg3l1 or Afg3l2 did not significantly affect OPA1 processing compared to expression of Afg3l1 and Afg3l2 alone (data not shown). To examine whether also hetero-oligomeric murine m-AAA proteases containing paraplegin can cleave OPA1, the previous observation that hetero-oligomeric m-AAA proteases containing both proteolytically active and inactive subunits are capable of mediating protein processing (Arlt 1998 Embo J 17, 4837-4847; Koppen loc cit) was exploited. Paraplegin or its proteolytically inactive variant was expressed in Δyta10Δyta12 yeast cells harboring the OPA1 splice variants 7 together with either proteolytically inactive Afg3l1^{E567Q} or Afg3l2^{E574Q}. Slight OPA1 processing, in particular formation of S3, was detectable with both the Afg3l2^{E567Q}/paraplegin complex and the Afg3l1^{E567Q}/paraplegin complex (FIG. 15). A similar dependency was observed for splice variant 8 (FIG. 15). In contrast, splice variant 4 was processed only to a very minor extent by the Afg3l2^{E567Q}/paraplegin complex but not by the Afg3l1^{E567Q}/paraplegin complex (FIG. 15). As small OPA1 isoforms were not detected when both, Afg 3l1 and paraplegin, or Afg 3l2 and paraplegin, contained point mutations in their proteolytic centres, the low proteolytic activity observed can be attributed to paraplegin. This indicates that hetero-oligomeric Afg 3l1^{E567Q}/paraplegin and Afg 3l2^{E567Q}/paraplegin complexes are able to cleave OPA1 weakly. However, this demonstrates that the processing efficiency by paraplegin containing m-AAA protease isozymes is rather limited, lower than that of homooligomeric Afg 3l1 or Afg 3l2 complexes, and varies for different splice variants. Taken together, OPA1 is predominantly processed by homomeric m-AAA protease complexes comprising Afg 3l1 or Afg 3l2.

The present invention refers to the following Tables:

TABLE 1
Plasmids.
Plasmids used and corresponding figure numbers are described below.

| Name | Description | Vector | Reference |
|---|---|---|---|
| pYES2-OPA1 (URA3) | OPA1 cDNA expression constructs for | pYES2 (Invitrogen) | this study |
| | splice variants 4, 7 or 8 | | |
| pYES2-OPA1 (ura3::TRP1) | OPA1 cDNA expression constructs for | pYES2 (Invitrogen) | this study |
| | splice variants 4, 7 or 8 | | |
| pCAG-OPA1-SP7-IRES- | OPA1 splice variant 7 cDNA for | pCAG-IRES | this study |
| mDsRed | overexpression in mammalian cells | | |
| pES425-PARL | PARL cDNA expression construct | pES425#1 (Doron | this study |
| | | Rapaport) | |
| pYES2-Pcp1 | Pcp1 expression construct | pYES2 (Invitrogen) | this study |
| pVT100U-mtGFP | Fluorescence microscopy | pVT100U | (Westermann and |
| | | | Neupert, 2000) |
| pRS314^{ADH1}-Yta10 (1-61)- | expression construct for human | pRS314 (Sikorski and | this study |
| hAFG3L2 (36-798)-Myc | AFG3L2 | Hieter, 1989) | |
| Ycplac111^{ADH1}-Yta10 (1-63)- | expression construct for human | Ycplac111 (Gietz and | (Atorino et al., 2003) |
| hparaplegin (59-795)-HA, | paraplegin | Sugino, 1988) | |
| Yeplac112^{ADH1}-Yta10 (1-61)- | expression construct for human | Yeplac112 (Gietz and | (Koppen et al., |
| hAFG3L2 (36-798)-Myc | AFG3L2 | Sugino, 1988) | 2007) |
| Yeplac112^{ADH1}-Yta10 (1-61)- | expression construct for proteolytically | Yeplac112 (Gietz and | (Koppen et al., |
| hAFG3L2^{E575Q} (36-798)-Myc | inactive hAFG3L2 | Sugino, 1988) | 2007) |
| Yeplac181^{YTA10}-Yta10 (1- | expression construct for murine | Yeplac181 (Gietz and | (Nolden et al., 2005) |
| 61)-paraplegin (44-781) | paraplegin | Sugino, 1988) | |
| Yeplac181^{YTA10}-Yta10 (1- | expression construct for proteolytically | Yeplac181 (Gietz and | (Koppen et al., |
| 61)-paraplegin^{E575Q} (44-781) | inactive paraplegin | Sugino, 1988) | 2007) |
| Yeplac195^{YTA10}-Yta10 (1- | expression construct for murine Afg3I1 | Yeplac195 (Gietz and | (Koppen et al., |
| 61)-Afg3I1 (25-789)-Myc | | Sugino, 1988) | 2007) |
| Yeplac195^{YTA10}-Yta10 (1- | expression construct for proteolytically | Yeplac195 (Gietz and | (Koppen et al., |
| 61)-Afg3I1^{E567Q} (25-789)- | inactive Afg3I1 | Sugino, 1988) | 2007) |
| Myc | | | |
| Yeplac112^{YTA10}-Yta10 (1- | expression construct for murine Afg3I2 | Yeplac112 (Gietz and | (Nolden et al., 2005) |
| 61)-Afg3I2 (36-802)-HA | | Sugino, 1988) | |
| Yeplac112^{YTA10}-Yta10 (1- | expression construct for proteolytically | Yeplac112 (Gietz and | (Koppen et al., |
| 61)-Afg3I2^{E574Q} (36-802)-HA | inactive Afg3I2 | Sugino, 1988) | 2007) |

TABLE 2
Strains.
The following strains were transformed with plasmids encoding OPA1 splice variants
as indicated in the text and figures.

| Description | Name | Background | Plasmids | Reference |
|---|---|---|---|---|
| WT | | W303α | | (Rothstein and |
| | | W303a | | Sherman, 1980) |
| PCP1/Δpcp1 | | BY4743 | | Euroscarf acc. No. |
| | | | | Y24731 |
| Δpcp1 + PCP1 | jw1-4c | spore of | pYES2-Pcp1 | this study |
| | | PCP1/Δpcp1 | | |
| PCP1 | jw2-1a | spore of | | this study |
| | | PCP1/Δpcp1 | | |
| Δpcp1 + PARL | jw2-6c | spore of | pES425-PARL | this study |
| | | PCP1/Δpcp1 | | |
| Δpcp1 | jw2-1c | spore of | | this study |
| | | PCP1/Δpcp1 | | |
| Δyta10Δyta12 | YKO100 | W303α | | (Atorino et al., 2003) |
| Δyta10Δyta12 + | | YKO100 | pES425-PARL | this study |
| PARL | | | | |
| Δyta10Δyta12 + | YKO117 | W303α | Ycplac111^{ADH1}-Yta10 (1-63)-hparaplegin (59- | (Atorino et al., 2003) |
| hAFG3L2 + | | | 795)-HA, pRS316^{ADH1}-Yta10 (1-61)- | |
| hparaplegin(LEU2) | | | hAFG3L2 (36-798)-Myc | |
| Δyta10Δyta12 + | jw8-1 | YKO117 | Ycplac111^{ADH1}-Yta10 (1-63)-hparaplegin (59- | this study |
| hAFG3L2 + | | | 795)-HA, pRS314^{ADH1}-Yta10 (1-61)- | |
| hparaplegin(TRP1) | | | hAFG3L2 (36-798)-Myc | |
| Δyta10Δyta12 | YKO200 | W303α | | (Koppen et al., |
| | | | | 2007) |
| Δyta10Δyta12 + | YKO203 | YKO200 | Yeplac112^{ADH1}-Yta10 (1-61)-hAFG3L2 (36- | (Koppen et al., |
| hAFG3L2 | | | 798)-Myc | 2007) |
| Δyta10Δyta12 + | YKO204 | YKO200 | Yeplac112^{ADH1}-Yta10 (1-61)-hAFG3L2^{E575Q} | (Koppen et al., |
| hAFG3L2^{EQ} | | | (36-798)-Myc | 2007) |
| Δyta10Δyta12 + | YKO209 | YKO200 | Yeplac181^{YTA10}-Yta10 (1-61)-paraplegin (44- | (Koppen et al., |
| paraplegin | | | 781) | 2007) |
| Δyta10Δyta12 + | YKO211 | YKO200 | Yeplac195^{YTA10}-Yta10 (1-61)-Afg3I1 (25- | (Koppen et al., |
| Afg3I1 | | | 789)-Myc | 2007) |
| Δyta10Δyta12 + | YKO212 | YKO200 | Yeplac195^{YTA10}-Yta10 (1-61)-Afg3I1^{E567Q} (25- | (Koppen et al., |
| Afg3I1^{EQ} | | | 789)-Myc | 2007) |
| Δyta10Δyta12 + | YKO213 | YKO200 | Yeplac112^{YTA10}-Yta10 (1-61)-Afg3I2 (36- | (Koppen et al., |
| Afg3I2 | | | 802)-HA | 2007) |
| Δyta10Δyta12 + | YKO214 | YKO200 | Yeplac112^{YTA10}-Yta10 (1-61)-Afg3I2^{E574Q} (36- | (Koppen et al., |
| Afg3I2^{EQ} | | | 802)-HA | 2007) |
| Δyta10Δyta12 + | YKO217 | YKO200 | Yeplac181^{YTA10}-Yta10 (1-61)-paraplegin (44- | (Koppen et al., |
| paraplegin + | | | 781), Yeplac195^{YTA10}-Yta10 (1-61)- | 2007) |
| Afg3I1^{EQ} | | | Afg3I1^{E567Q} (25-789)-Myc | |
| Δyta10Δyta12 + | YKO218 | YKO200 | Yeplac181^{YTA10}-Yta10 (1-61)-paraplegin^{E575Q} + | (Koppen et al., |
| paraplegin^{EQ} + | | | (44-781), Yeplac195^{YTA10}-Yta10 (1-61)- | 2007) |
| Afg3I1^{EQ} | | | AfgI1^{E567Q} (25-789)-Myc | |
| Δyta10Δyta12 + | YKO221 | YKO200 | Yeplac181^{YTA10}-Yta10 (1-61)-paraplegin (44- | (Koppen et al., |
| paraplegin + | | | 781), Yeplac112^{YTA10}-Yta10 (1-61)- | 2007) |
| Afg3I2^{EQ} | | | Afg3I2^{E574Q} (36-802)-HA | |
| Δyta10Δyta12 + | YKO222 | YKO200 | Yeplac181^{YTA10}-Yta10 (1-61)-paraplegin^{E575Q} | (Koppen et al., |
| paraplegin^{EQ} + | | | (44-781), Yeplac112^{YTA10}-Yta10 (1-61)- | 2007) |
| Afg3I2^{EQ} | | | Afg3I2^{E574Q} (36-802)-HA | |

References cited in Table 1 and 2:
Atorino, L., Silvestri, L., Koppen, M., Cassina, L., Ballabio, A., Marconi, R., Langer, T., and Casari, G. (2003). Loss of m-AAA protease in mitochondria causes complex I deficiency and increased sensitivity to oxidative stress in hereditary spastic paraplegia. J Cell Biol 163, 777-787.
Gietz, R. D., and Sugino, A. (1988). New yeast-Escherichia coli shuttle vectors constructed with in vitro mutagenized yeast genes lacking six-base pair restriction sites. Gene 74, 527-534.
Koppen, M., Metodiev, M. D., Casari, G., Rugarli, E. I., and Langer, T. (2007). Variable and tissue-specific subunit composition of mitochondrial m-AAA protease complexes linked to hereditary spastic paraplegia. Mol. Cell. Biol. in press.
Nolden, M., Ehses, S., Koppen, M., Bernacchia, A., Rugarli, E. I., and Langer, T. (2005). The m-AAA protease defective in hereditary spastic paraplegia controls ribosome assembly in mitochondria. Cell 123, 277-289.
Rothstein, R. J., and Sherman, F. (1980). Genes affecting the expression of cytochrome c in yeast: genetic mapping and genetic interactions. Genetics 94, 871-889.
Sikorski, R. S., and Hieter, P. (1989). A system of shuttle vectors and yeast host strains designed for efficient manipulation of DNA in Saccharomyces cerevisiae. Genetics 122, 19-27.
Westermann, B., and Neupert, W. (2000). Mitochondria-targeted green fluorescent proteins: convenient tools for the study of organelle biogenesis in Saccharomyces cerevisiae. Yeast 16, 1421-1427.

The present invention refers to the following nucleotide and amino acid sequences:

| SEQ ID No. 1: | |
|---|---|
| Nucleotide sequence encoding OPA1 spliceform 8 | |
| (NM_130837; CDS 56-3103) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttgagaaaat tagaaaagcc cttcctaatt cagaagacct |
| 541 | tgtaaagtta gcaccagact ttgacaagat tgttgaaagc cttagcttat tgaaggactt |
| 601 | ttttacctca ggtcacaaat tggttagtga agtcatagga gcttctgacc tacttctctt |
| 661 | gttaggttct ccggaagaaa cggcgtttag agcaacagat cgtggatctg aaagtgacaa |
| 721 | gcattttaga aagggtctgc ttggtgagct cattctctta caacaacaaa ttcaagagca |
| 781 | tgaagaggaa gcgcgcagag ccgctggcca atatagcacg agctatgccc aacagaagcg |
| 841 | caaggtgtca gacaaagaga aaattgacca acttcaggaa gaacttctgc acactcagtt |
| 901 | gaagtatcag agaatcttgg aacgattaga aaaggagaac aaagaattga gaaaattagt |
| 961 | attgcagaaa gatgacaaag gcattcatca tagaaagctt aagaaatctt tgattgacat |
| 1021 | gtattctgaa gttcttgatg ttctctctga ttatgatgcc agttataata cgcaagatca |
| 1081 | tctgccacgg gttgttgtgg ttggagatca gagtgctgga aagactagtg tgttggaaat |
| 1141 | gattgcccaa gctcgaatat tcccaagagg atctggggag atgatgacac gttctccagt |
| 1201 | taaggtgact ctgagtgaag gtcctcacca tgtggcccta tttaaagata gttctcggga |
| 1261 | gtttgatctt accaaagaag aagatcttgc agcattaaga catgaaatag aacttcgaat |
| 1321 | gaggaaaaat gtgaaagaag gctgtaccgt tagccctgag accatatcct taaatgtaaa |
| 1381 | aggccctgga ctacagagga tggtgcttgt tgacttacca ggtgtgatta atactgtgac |
| 1441 | atcaggcatg gctcctgaca caaaggaaac tattttcagt atcagcaaag cttacatgca |
| 1501 | gaatcctaat gccatcatac tgtgtattca agatggatct gtggatgctg aacgcagtat |
| 1561 | tgttacagac ttggtcagtc aaatggaccc tcatggaagg agaaccatat tcgttttgac |
| 1621 | caaagtagac ctggcagaga aaaatgtagc cagtccaagc aggattcagc agataattga |
| 1681 | aggaaagctc ttcccaatga aagctttagg ttattttgct gttgtaacag gaaaagggaa |
| 1741 | cagctctgaa agcattgaag ctataagaga atatgaagaa gagttttttc agaattcaaa |
| 1801 | gctcctaaag acaagcatgc taaaggcaca ccaagtgact acaagaaatt taagccttgc |
| 1861 | agtatcagac tgcttttgga aaatggtacg agagtctgtt gaacaacagg ctgatagttt |
| 1921 | caaagcaaca cgttttaacc ttgaaactga atggaagaat aactatcctc gcctgcggga |
| 1981 | acttgaccgg aatgaactat ttgaaaaagc taaaaatgaa atccttgatg aagttatcag |
| 2041 | tctgagccag gttacaccaa aacattggga ggaaatcctt caacaatctt tgtgggaaag |
| 2101 | agtatcaact catgtgattg aaaacatcta ccttccagct gcgcagacca tgaattcagg |
| 2161 | aacttttaac accacagtgg atatcaagct taaacagtgg actgataaac aacttcctaa |
| 2221 | taaagcagta gaggttgctt gggagaccct acaagaagaa ttttcccgct ttatgacaga |
| 2281 | accgaaaggg aaagagcatg atgacatatt tgataaactt aaagaggccg ttaaggaaga |
| 2341 | aagtattaaa cgacacaagt ggaatgactt tgcggaggac agcttgaggg ttattcaaca |
| 2401 | caatgctttg gaagaccgat ccatatctga taaacagcaa tgggatgcag ctatttattt |
| 2461 | tatggaagag gctctgcagg ctcgtctcaa ggatactgaa aatgcaattg aaaacatggt |
| 2521 | gggtccagac tggaaaaaga ggtggttata ctggaagaat cggacccaag aacagtgtgt |
| 2581 | tcacaatgaa accaagaatg aattggagaa gatgttgaaa tgtaatgagg agcacccagc |
| 2641 | ttatcttgca agtgatgaaa taaccacagt ccggaagaac cttgaatccc gaggagtaga |
| 2701 | agtagatcca agcttgatta aggatacttg gcatcaagtt tatagaagac attttttaaa |
| 2761 | aacagctcta aaccattgta acctttgtcg aagaggtttt tattactacc aaaggcattt |
| 2821 | tgtagattct gagttggaat gcaatgatgt ggtcttgttt tggcgtatac agcgcatgct |
| 2881 | tgctatcacc gcaaatactt taaggcaaca acttacaaat actgaagtta ggcgattaga |
| 2941 | gaaaaatgtt aaagaggtat tggaagattt tgctgaagat ggtgagaaga agattaaatt |
| 3001 | gcttactggt aaacgcgttc aactggcgga agacctcaag aaagttagag aaattcaaga |
| 3061 | aaaacttgat gctttcattg aagctcttca tcaggagaaa taa |
| SEQ ID No. 2: | |
| Amino acid sequence of OPA1 spliceform 8 (NP_570850; 1015 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfe kirkalpnse dlvklapdfd kiveslsllk |
| 181 | dfftsghklv sevigasdll lllgspeeta fratdrgses dkhfrkgllg elillqqqiq |
| 241 | eheeearraa gqystsyaqq krkvsdkeki dqlqeellht qlkyqriler lekenkelrk |
| 301 | lvlqkddkgi hhrklkksli dmysevldvl sdydasyntq dhlprvvvvg dqsagktsvl |
| 361 | emiaqarifp rgsgemmtrs pvkvtlsegp hhvalfkdss refdltkeed laalrheiel |
| 421 | rmrknvkegc tvspetisln vkgpglqrmv lvdlpgvint vtsgmapdtk etifsiskay |
| 481 | mqnpnaiilc iqdgsvdaer sivtdlvsqm dphgrrtifv ltkvdlaekn vaspsriqqi |
| 541 | iegklfpmka lgyfavvtgk gnssesieai reyeeeffqn skllktsmlk ahqvttrnls |
| 601 | lavsdcfwkm vresveqqad sfkatrfnle tewknnyprl reldrnelfe kakneildev |
| 661 | islsqvtpkh weeilqqslw ervsthvien iylpaaqtmn sgtfnttvdi klkqwtdkql |
| 721 | pnkavevawe tlqeefsrfm tepkgkehdd ifdklkeavk eesikrhkwn dfaedslrvi |
| 781 | qhnaledrsi sdkqqwdaai yfmeealqar lkdtenaien mvgpdwkkrw lywknrtqeq |
| 841 | cvhnetknel ekmlkcneeh paylasdeit tvrknlesrg vevdpslikd twhqvyrrhf |
| 901 | lktalnhcnl crrgfyyyqr hfvdselecn dvvlfwriqr mlaitantlr qqltntevrr |
| 961 | leknvkevle dfaedgekki klltgkrvql aedlkkvrei qekldafiea lhqek |
| SEQ ID No. 3: | |
| Nucleotide sequence encoding OPA1 spliceform 7 | |
| (NM_130836; CDS 56-3049) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttgagaaaat tagaaaagcc cttcctaatt cagaagacct |
| 541 | tgtaaagtta gcaccagact ttgacaagat tgttgaaagc cttagcttat tgaaggactt |
| 601 | ttttacctca ggttctccgg aagaaacggc gtttagagca acagatcgtg gatctgaaag |
| 661 | tgacaagcat tttagaaagg gtctgcttgg tgagctcatt ctcttacaac aacaaattca |
| 721 | agagcatgaa gaggaagcgc gcagagccgc tggccaatat agcacgagct atgcccaaca |
| 781 | gaagcgcaag gtgtcagaca aagagaaaat tgaccaactt caggaagaac ttctgcacac |
| 841 | tcagttgaag tatcagagaa tcttggaacg attagaaaag gagaacaaag aattgagaaa |
| 901 | attagtattg cagaaagatg acaaaggcat tcatcataga aagcttaaga aatctttgat |
| 961 | tgacatgtat tctgaagttc ttgatgttct ctctgattat gatgccagtt ataatacgca |
| 1021 | agatcatctg ccacgggttg ttgtggttgg agatcagagt gctggaaaga ctagtgtgtt |
| 1081 | ggaaatgatt gcccaagctc gaatattccc aagaggatct ggggagatga tgacacgttc |
| 1141 | tccagttaag gtgactctga gtgaaggtcc tcaccatgtg gccctattta aagatagttc |
| 1201 | tcgggagttt gatcttacca aagaagaaga tcttgcagca ttaagacatg aaatagaact |
| 1261 | tcgaatgagg aaaaatgtga aagaaggctg taccgttagc cctgagacca tatccttaaa |
| 1321 | tgtaaaaggc cctggactac agaggatggt gcttgttgac ttaccaggtg tgattaatac |
| 1381 | tgtgacatca ggcatggctc ctgacacaaa ggaaactatt ttcagtatca gcaaagctta |
| 1441 | catgcagaat cctaatgcca tcatactgtg tattcaagat ggatctgtgg atgctgaacg |
| 1501 | cagtattgtt acagacttgg tcagtcaaat ggaccctcat ggaaggagaa ccatattcgt |
| 1561 | tttgaccaaa gtagacctgg cagagaaaaa tgtagccagt ccaagcagga ttcagcagat |
| 1621 | aattgaagga aagctcttcc caatgaaagc tttaggttat tttgctgttg taacaggaaa |
| 1681 | agggaacagc tctgaaagca ttgaagctat aagagaatat gaagaagagt tttttcagaa |
| 1741 | ttcaaagctc ctaaagacaa gcatgctaaa ggcacaccaa gtgactacaa gaaatttaag |
| 1801 | ccttgcagta tcagactgct tttggaaaat ggtacgagag tctgttgaac aacaggctga |
| 1861 | tagtttcaaa gcaacacgtt ttaaccttga aactgaatgg aagaataact atcctcgcct |
| 1921 | gcgggaactt gaccggaatg aactatttga aaaagctaaa aatgaaatcc ttgatgaagt |
| 1981 | tatcagtctg agccaggtta caccaaaaca ttgggaggaa atccttcaac aatctttgtg |
| 2041 | ggaaagagta tcaactcatg tgattgaaaa catctacctt ccagctgcgc agaccatgaa |
| 2101 | ttcaggaact tttaacacca cagtggatat caagcttaaa cagtggactg ataaacaact |
| 2161 | tcctaataaa gcagtagagg ttgcttggga gaccctacaa gaagaatttt cccgctttat |
| 2221 | gacagaaccg aaagggaaag agcatgatga catatttgat aaacttaaag aggccgttaa |
| 2281 | ggaagaaagt attaaacgac acaagtggaa tgactttgcg gaggacagct tgagggttat |
| 2341 | tcaacacaat gctttggaag accgatccat atctgataaa cagcaatggg atgcagctat |
| 2401 | ttattttatg gaagaggctc tgcaggctcg tctcaaggat actgaaaatg caattgaaaa |
| 2461 | catggtgggt ccagactgga aaaagaggtg gttatactgg aagaatcgga cccaagaaca |
| 2521 | gtgtgttcac aatgaaacca agaatgaatt ggagaagatg ttgaaatgta atgaggagca |
| 2581 | cccagcttat cttgcaagtg atgaaataac cacagtccgg aagaaccttg aatcccgagg |
| 2641 | agtagaagta gatccaagct tgattaagga tacttggcat caagtttata gaagacattt |
| 2701 | tttaaaaaca gctctaaacc attgtaacct ttgtcgaaga ggtttttatt actaccaaag |
| 2761 | gcattttgta gattctgagt tggaatgcaa tgatgtggtc ttgttttggc gtatacagcg |
| 2821 | catgcttgct atcaccgcaa atactttaag gcaacaactt acaaatactg aagttaggcg |
| 2881 | attagagaaa aatgttaaag aggtattgga agattttgct gaagatggtg agaagaagat |
| 2941 | taaattgctt actggtaaac gcgttcaact ggcggaagac ctcaagaaag ttagagaaat |
| 3001 | tcaagaaaaa cttgatgctt tcattgaagc tcttcatcag gagaaataa |
| SEQ ID No. 4: | |
| Amino acid sequence of OPA1 spliceform 7 (NP_570849; 997 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfe kirkalpnse dlvklapdfd kiveslsllk |
| 181 | dfftsgspee tafratdrgs esdkhfrkgl lgelillqqq iqeheeearr aagqystsya |
| 241 | qqkrkvsdke kidqlqeell htqlkyqril erlekenkel rklvlqkddk gihhrklkks |
| 301 | lidmysevld vlsdydasyn tqdhlprvvv vgdqsagkts vlemiaqari fprgsgemmt |
| 361 | rspvkvtlse gphhvalfkd ssrefdltke edlaalrhei elrmrknvke gctvspetis |
| 421 | lnvkgpglqr mvlvdlpgvi ntvtsgmapd tketifsisk aymqnpnaii lciqdgsvda |
| 481 | ersivtdlvs qmdphgrrti fvltkvdlae knvaspsriq qiiegklfpm kalgyfavvt |
| 541 | gkgnssesie aireyeeeff qnskllktsm lkahqvttrn lslavsdcfw kmvresveqq |
| 601 | adsfkatrfn letewknnyp rlreldrnel fekakneild evislsqvtp khweeilqqs |
| 661 | lwervsthvi eniylpaaqt mnsgtfnttv diklkqwtdk qlpnkaveva wetlqeefsr |
| 721 | fmtepkgkeh ddifdklkea vkeesikrhk wndfaedslr viqhnaledr sisdkqqwda |
| 781 | aiyfmeealq arlkdtenai enmvgpdwkk rwlywknrtq eqcvhnetkn elekmlkcne |
| 841 | ehpaylasde ittvrknles rgvevdpsli kdtwhqvyrr hflktalnhc nlcrrgfyyy |
| 901 | qrhfvdsele cndvvlfwri qrmlaitant lrqqltntev rrleknvkev ledfaedgek |
| 961 | kiklltgkrv qlaedlkkvr eiqekldafi ealhqek |
| SEQ ID No. 5: | |
| Nucleotide sequence encoding OPA1 spliceform 6 | |
| (NM_130835; CDS 56-2995) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttggtcacaa attggttagt gaagtcatag gagcttctga |
| 541 | cctacttctc ttgttaggtt ctccggaaga aacggcgttt agagcaacag atcgtggatc |
| 601 | tgaaagtgac aagcatttta gaaagggtct gcttggtgag ctcattctct tacaacaaca |
| 661 | aattcaagag catgaagagg aagcgcgcag agccgctggc caatatagca cgagctatgc |
| 721 | ccaacagaag cgcaaggtgt cagacaaaga gaaaattgac caacttcagg aagaacttct |
| 781 | gcacactcag ttgaagtatc agagaatctt ggaacgatta gaaaaggaga acaaagaatt |
| 841 | gagaaaatta gtattgcaga aagatgacaa aggcattcat catagaaagc ttaagaaatc |
| 901 | tttgattgac atgtattctg aagttcttga tgttctctct gattatgatg ccagttataa |
| 961 | tacgcaagat catctgccac gggttgttgt ggttggagat cagagtgctg gaaagactag |
| 1021 | tgtgttggaa atgattgccc aagctcgaat attcccaaga ggatctgggg agatgatgac |
| 1081 | acgttctcca gttaaggtga ctctgagtga aggtcctcac catgtggccc tatttaaaga |
| 1141 | tagttctcgg gagtttgatc ttaccaaaga agaagatctt gcagcattaa gacatgaaat |
| 1201 | agaacttcga atgaggaaaa atgtgaaaga aggctgtacc gttagccctg agaccatatc |
| 1261 | cttaaatgta aaaggccctg gactacagag gatggtgctt gttgacttac caggtgtgat |
| 1321 | taatactgtg acatcaggca tggctcctga cacaaaggaa actattttca gtatcagcaa |
| 1381 | agcttacatg cagaatccta atgccatcat actgtgtatt caagatggat ctgtggatgc |
| 1441 | tgaacgcagt attgttacag acttggtcag tcaaatggac cctcatggaa ggagaaccat |
| 1501 | attcgttttg accaaagtag acctggcaga gaaaaatgta gccagtccaa gcaggattca |
| 1561 | gcagataatt gaaggaaagc tcttcccaat gaaagcttta ggttattttg ctgttgtaac |
| 1621 | aggaaaaggg aacagctctg aaagcattga agctataaga gaatatgaag aagagttttt |
| 1681 | tcagaattca aagctcctaa agacaagcat gctaaaggca caccaagtga ctacaagaaa |
| 1741 | tttaagcctt gcagtatcag actgcttttg gaaaatggta cgagagtctg ttgaacaaca |
| 1801 | ggctgatagt ttcaaagcaa cacgttttaa ccttgaaact gaatggaaga ataactatcc |
| 1861 | tcgcctgcgg gaacttgacc ggaatgaact atttgaaaaa gctaaaaatg aaatccttga |
| 1921 | tgaagttatc agtctgagcc aggttacacc aaaacattgg gaggaaatcc ttcaacaatc |
| 1981 | tttgtgggaa agagtatcaa ctcatgtgat tgaaaacatc taccttccag ctgcgcagac |
| 2041 | catgaattca ggaactttta acaccacagt ggatatcaag cttaaacagt ggactgataa |
| 2101 | acaacttcct aataaagcag tagaggttgc ttgggagacc ctacaagaag aattttcccg |
| 2161 | ctttatgaca gaaccgaaag ggaaagagca tgatgacata tttgataaac ttaaagaggc |
| 2221 | cgttaaggaa gaaagtatta aacgacacaa gtggaatgac tttgcggagg acagcttgag |
| 2281 | ggttattcaa cacaatgctt tggaagaccg atccatatct gataaacagc aatgggatgc |
| 2341 | agctatttat tttatggaag aggctctgca ggctcgtctc aaggatactg aaaatgcaat |
| 2401 | tgaaaacatg gtgggtccag actggaaaaa gaggtggtta tactggaaga atcggaccca |
| 2461 | agaacagtgt gttcacaatg aaaccaagaa tgaattggag aagatgttga aatgtaatga |
| 2521 | ggagcaccca gcttatcttg caagtgatga aataaccaca gtccggaaga accttgaatc |
| 2581 | ccgaggagta gaagtagatc caagcttgat taaggatact tggcatcaag tttatagaag |
| 2641 | acatttttta aaaacagctc taaaccattg taacctttgt cgaagaggtt tttattacta |
| 2701 | ccaaaggcat tttgtagatt ctgagttgga atgcaatgat gtggtcttgt tttggcgtat |
| 2761 | acagcgcatg cttgctatca ccgcaaatac tttaaggcaa caacttacaa atactgaagt |
| 2821 | taggcgatta gagaaaaatg ttaaagaggt attggaagat tttgctgaag atggtgagaa |
| 2881 | gaagattaaa ttgcttactg gtaaacgcgt tcaactggcg gaagacctca agaaagttag |
| 2941 | agaaattcaa gaaaaacttg atgctttcat tgaagctctt catcaggaga aataa |
| SEQ ID No. 6: | |
| Amino acid sequence of OPA1 spliceform 6 (NP_570848; 979 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfg hklvseviga sdlllllgsp eetafratdr |
| 181 | gsesdkhfrk gllgelillq qqiqeheeea rraagqysts yaqqkrkvsd kekidqlqee |
| 241 | llhtqlkyqr ilerlekenk elrklvlqkd dkgihhrklk kslidmysev ldvlsdydas |
| 301 | yntqdhlprv vvvgdqsagk tsvlemiaqa rifprgsgem mtrspvkvtl segphhvalf |
| 361 | kdssrefdlt keedlaalrh eielrmrknv kegctvspet islnvkgpgl qrmvlvdlpg |
| 421 | vintvtsgma pdtketifsi skaymqnpna iilciqdgsv daersivtdl vsqmdphgrr |
| 481 | tifvltkvdl aeknvaspsr iqqiiegklf pmkalgyfav vtgkgnsses ieaireyeee |
| 541 | ffqnskllkt smlkahqvtt rnlslavsdc fwkmvresve qqadsfkatr fnletewknn |
| 601 | yprlreldrn elfekaknei ldevislsqv tpkhweeilq qslwervsth vieniylpaa |
| 661 | qtmnsgtfnt tvdiklkqwt dkqlpnkave vawetlqeef srfmtepkgk ehddifdklk |
| 721 | eavkeesikr hkwndfaeds lrviqhnale drsisdkqqw daaiyfmeea lqarlkdten |
| 781 | aienmvgpdw kkrwlywknr tqeqcvhnet knelekmlkc neehpaylas deittvrknl |
| 841 | esrgvevdps likdtwhqvy rrhflktaln hcnlcrrgfy yyqrhfvdse lecndvvlfw |
| 901 | riqrmlaita ntlrqqltnt evrrleknvk evledfaedg ekkiklltgk rvqlaedlkk |
| 961 | vreiqeklda fiealhqek |
| SEQ ID No. 7: | |
| Nucleotide sequence encoding OPA1 spliceform 5 | |
| (NM_130834; CDS 56-2992) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttgagaaaat tagaaaagcc cttcctaatt cagaagacct |
| 541 | tgtaaagtta gcaccagact ttgacaagat tgttgaaagc cttagcttat tgaaggactt |
| 601 | ttttacctca ggtcacaaat tggttagtga agtcatagga gcttctgacc tacttctctt |
| 661 | gttaggttct ccggaagaaa cggcgtttag agcaacagat cgtggatctg aaagtgacaa |
| 721 | gcattttaga aaggtgtcag acaaagagaa aattgaccaa cttcaggaag aacttctgca |
| 781 | cactcagttg aagtatcaga gaatcttgga acgattagaa aaggagaaca aagaattgag |
| 841 | aaaattagta ttgcagaaag atgacaaagg cattcatcat agaaagctta agaaatcttt |
| 901 | gattgacatg tattctgaag ttcttgatgt tctctctgat tatgatgcca gttataatac |
| 961 | gcaagatcat ctgccacggg ttgttgtggt tggagatcag agtgctggaa agactagtgt |
| 1021 | gttggaaatg attgcccaag ctcgaatatt cccaagagga tctggggaga tgatgacacg |
| 1081 | ttctccagtt aaggtgactc tgagtgaagg tcctcaccat gtggccctat ttaaagatag |
| 1141 | ttctcgggag tttgatctta ccaaagaaga agatcttgca gcattaagac atgaaataga |
| 1201 | acttcgaatg aggaaaaatg tgaaagaagg ctgtaccgtt agccctgaga ccatatcctt |
| 1261 | aaatgtaaaa ggccctggac tacagaggat ggtgcttgtt gacttaccag gtgtgattaa |
| 1321 | tactgtgaca tcaggcatgg ctcctgacac aaaggaaact attttcagta tcagcaaagc |
| 1381 | ttacatgcag aatcctaatg ccatcatact gtgtattcaa gatggatctg tggatgctga |
| 1441 | acgcagtatt gttacagact tggtcagtca aatggaccct catggaagga gaaccatatt |
| 1501 | cgttttgacc aaagtagacc tggcagagaa aaatgtagcc agtccaagca ggattcagca |
| 1561 | gataattgaa ggaaagctct tcccaatgaa agctttaggt tattttgctg ttgtaacagg |
| 1621 | aaaagggaac agctctgaaa gcattgaagc tataagagaa tatgaagaag agttttttca |
| 1681 | gaattcaaag ctcctaaaga caagcatgct aaaggcacac caagtgacta caagaaattt |
| 1741 | aagccttgca gtatcagact gcttttggaa aatggtacga gagtctgttg aacaacaggc |
| 1801 | tgatagtttc aaagcaacac gttttaacct tgaaactgaa tggaagaata actatcctcg |
| 1861 | cctgcgggaa cttgaccgga atgaactatt tgaaaaagct aaaaatgaaa tccttgatga |
| 1921 | agttatcagt ctgagccagg ttacaccaaa acattgggag gaaatccttc aacaatcttt |
| 1981 | gtgggaaaga gtatcaactc atgtgattga aaacatctac cttccagctg cgcagaccat |
| 2041 | gaattcagga acttttaaca ccacagtgga tatcaagctt aaacagtgga ctgataaaca |
| 2101 | acttcctaat aaagcagtag aggttgcttg ggagacccta caagaagaat tttcccgctt |
| 2161 | tatgacagaa ccgaaaggga aagagcatga tgacatattt gataaactta aagaggccgt |
| 2221 | taaggaagaa agtattaaac gacacaagtg gaatgacttt gcggaggaca gcttgagggt |
| 2281 | tattcaacac aatgctttgg aagaccgatc catatctgat aaacagcaat gggatgcagc |
| 2341 | tatttatttt atggaagagg ctctgcaggc tcgtctcaag gatactgaaa atgcaattga |
| 2401 | aaacatggtg ggtccagact ggaaaaagag gtggttatac tggaagaatc ggacccaaga |
| 2461 | acagtgtgtt cacaatgaaa ccaagaatga attggagaag atgttgaaat gtaatgagga |
| 2521 | gcacccagct tatcttgcaa gtgatgaaat aaccacagtc cggaagaacc ttgaatcccg |
| 2581 | aggagtagaa gtagatccaa gcttgattaa ggatacttgg catcaagttt atagaagaca |
| 2641 | ttttttaaaa acagctctaa accattgtaa cctttgtcga agaggttttt attactacca |
| 2701 | aaggcatttt gtagattctg agttggaatg caatgatgtg gtcttgtttt ggcgtataca |
| 2761 | gcgcatgctt gctatcaccg caaatacttt aaggcaacaa cttacaaata ctgaagttag |
| 2821 | gcgattagag aaaaatgtta aagaggtatt ggaagatttt gctgaagatg gtgagaagaa |
| 2881 | gattaaattg cttactggta aacgcgttca actggcggaa gacctcaaga aagttagaga |
| 2941 | aattcaagaa aaacttgatg ctttcattga agctcttcat caggagaaat aa |
| SEQ ID No. 8: | |
| Amino acid sequence of OPA1 spliceform 5 (NP_570847; 978 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfe kirkalpnse dlvklapdfd kiveslsllk |
| 181 | dfftsghklv sevigasdll lllgspeeta fratdrgses dkhfrkvsdk ekidqlqeel |
| 241 | lhtqlkyqri lerlekenke lrklvlqkdd kgihhrklkk slidmysevl dvlsdydasy |
| 301 | ntqdhlprvv vvgdqsagkt svlemiaqar ifprgsgemm trspvkvtls egphhvalfk |
| 361 | dssrefdltk eedlaalrhe ielrmrknvk egctvspeti slnvkgpglq rmvlvdlpgv |
| 421 | intvtsgmap dtketifsis kaymqnpnai ilciqdgsvd aersivtdlv sqmdphgrrt |
| 481 | ifvltkvdla eknvaspsri qqiiegklfp mkalgyfavv tgkgnssesi eaireyeeef |
| 541 | fqnskllkts mlkahqvttr nlslavsdcf wkmvresveq qadsfkatrf nletewknny |
| 601 | prlreldrne lfekakneil devislsqvt pkhweeilqq slwervsthv ieniylpaaq |
| 661 | tmnsgtfntt vdiklkqwtd kqlpnkavev awetlqeefs rfmtepkgke hddifdklke |
| 721 | avkeesikrh kwndfaedsl rviqhnaled rsisdkqqwd aaiyfmeeal qarlkdtena |
| 781 | ienmvgpdwk krwlywknrt qeqcvhnetk nelekmlkcn eehpaylasd eittvrknle |
| 841 | srgvevdpsl ikdtwhqvyr rhflktalnh cnlcrrgfyy yqrhfvdsel ecndvvlfwr |
| 901 | iqrmlaitan tlrqqltnte vrrleknvke vledfaedqe kkiklltgkr vqlaedlkkv |
| 961 | reiqekldaf iealhqek |
| SEQ ID No. 9: | |
| Nucleotide sequence encoding OPA1 spliceform 4 | |
| (NM_130833; CDS 56-2941) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttggttctcc ggaagaaacg gcgtttagag caacagatcg |
| 541 | tggatctgaa agtgacaagc attttagaaa gggtctgctt ggtgagctca ttctcttaca |
| 601 | acaacaaatt caagagcatg aagaggaagc gcgcagagcc gctggccaat atagcacgag |
| 661 | ctatgcccaa cagaagcgca aggtgtcaga caaagagaaa attgaccaac ttcaggaaga |
| 721 | acttctgcac actcagttga agtatcagag aatcttggaa cgattagaaa aggagaacaa |
| 781 | agaattgaga aaattagtat tgcagaaaga tgacaaaggc attcatcata gaaagcttaa |
| 841 | gaaatctttg attgacatgt attctgaagt tcttgatgtt ctctctgatt atgatgccag |
| 901 | ttataatacg caagatcatc tgccacgggt tgttgtggtt ggagatcaga gtgctggaaa |
| 961 | gactagtgtg ttggaaatga ttgcccaagc tcgaatattc ccaagaggat ctggggagat |
| 1021 | gatgacacgt tctccagtta aggtgactct gagtgaaggt cctcaccatg tggccctatt |
| 1081 | taaagatagt tctcgggagt ttgatcttac caaagaagaa gatcttgcag cattaagaca |
| 1141 | tgaaatagaa cttcgaatga ggaaaaatgt gaaagaaggc tgtaccgtta gccctgagac |
| 1201 | catatcctta aatgtaaaag gccctggact acagaggatg gtgcttgttg acttaccagg |
| 1261 | tgtgattaat actgtgacat caggcatggc tcctgacaca aaggaaacta ttttcagtat |
| 1321 | cagcaaagct tacatgcaga atcctaatgc catcatactg tgtattcaag atggatctgt |
| 1381 | ggatgctgaa cgcagtattg ttacagactt ggtcagtcaa atggaccctc atggaaggag |
| 1441 | aaccatattc gttttgacca aagtagacct ggcagagaaa aatgtagcca gtccaagcag |
| 1501 | gattcagcag ataattgaag gaaagctctt cccaatgaaa gctttaggtt attttgctgt |
| 1561 | tgtaacagga aaagggaaca gctctgaaag cattgaagct ataagagaat atgaagaaga |
| 1621 | gttttttcag aattcaaagc tcctaaagac aagcatgcta aaggcacacc aagtgactac |
| 1681 | aagaaattta agccttgcag tatcagactg cttttggaaa atggtacgag agtctgttga |
| 1741 | acaacaggct gatagtttca aagcaacacg ttttaacctt gaaactgaat ggaagaataa |
| 1801 | ctatcctcgc ctgcgggaac ttgaccggaa tgaactattt gaaaaagcta aaaatgaaat |
| 1861 | ccttgatgaa gttatcagtc tgagccaggt tacaccaaaa cattgggagg aaatccttca |
| 1921 | acaatctttg tgggaaagag tatcaactca tgtgattgaa aacatctacc ttccagctgc |
| 1981 | gcagaccatg aattcaggaa cttttaacac cacagtggat atcaagctta aacagtggac |
| 2041 | tgataaacaa cttcctaata aagcagtaga ggttgcttgg gagaccctac aagaagaatt |
| 2101 | ttcccgcttt atgacagaac cgaaagggaa agagcatgat gacatatttg ataaacttaa |
| 2161 | agaggccgtt aaggaagaaa gtattaaacg acacaagtgg aatgactttg cggaggacag |
| 2221 | cttgagggtt attcaacaca atgctttgga agaccgatcc atatctgata aacagcaatg |
| 2281 | ggatgcagct atttatttta tggaagaggc tctgcaggct cgtctcaagg atactgaaaa |
| 2341 | tgcaattgaa aacatggtgg gtccagactg gaaaaagagg tggttatact ggaagaatcg |
| 2401 | gacccaagaa cagtgtgttc acaatgaaac caagaatgaa ttggagaaga tgttgaaatg |
| 2461 | taatgaggag cacccagctt atcttgcaag tgatgaaata accacagtcc ggaagaacct |
| 2521 | tgaatcccga ggagtagaag tagatccaag cttgattaag gatacttggc atcaagttta |
| 2581 | tagaagacat tttttaaaaa cagctctaaa ccattgtaac ctttgtcgaa gaggttttta |
| 2641 | ttactaccaa aggcattttg tagattctga gttggaatgc aatgatgtgg tcttgttttg |
| 2701 | gcgtatacag cgcatgcttg ctatcaccgc aaatacttta aggcaacaac ttacaaatac |
| 2761 | tgaagttagg cgattagaga aaaatgttaa agaggtattg gaagattttg ctgaagatgg |
| 2821 | tgagaagaag attaaattgc ttactggtaa acgcgttcaa ctggcggaag acctcaagaa |
| 2881 | agttagagaa attcaagaaa aacttgatgc tttcattgaa gctcttcatc aggagaaata |
| 2941 | a |
| SEQ ID No. 10: | |
| Amino acid sequence of OPA1 spliceform 4 (NP_570846; 961 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfg speetafrat drgsesdkhf rkgllgelil |
| 181 | lqqqiqehee earraagqys tsyaqqkrkv sdkekidqlq eellhtqlky qrilerleke |
| 241 | nkelrklvlq kddkgihhrk lkkslidmys evldvlsdyd asyntqdhlp rvvvvgdqsa |
| 301 | gktsvlemia qarifprgsg emmtrspvkv tlsegphhva lfkdssrefd ltkeedlaal |
| 361 | rheielrmrk nvkegctvsp etislnvkgp glqrmvlvdl pgvintvtsg mapdtketif |
| 421 | siskaymqnp naiilciqdg svdaersivt dlvsqmdphg rrtifvltkv dlaeknvasp |
| 481 | sriqqiiegk lfpmkalgyf avvtgkgnss esieaireye eeffqnskll ktsmlkahqv |
| 541 | ttrnlslavs dcfwkmvres veqqadsfka trfnletewk nnyprlreld rnelfekakn |
| 601 | eildevisls qvtpkhweei lqqslwervs thvieniylp aaqtmnsgtf nttvdiklkq |
| 661 | wtdkqlpnka vevawetlqe efsrfmtepk gkehddifdk lkeavkeesi krhkwndfae |
| 721 | dslrviqhna ledrsisdkq qwdaaiyfme ealqarlkdt enaienmvgp dwkkrwlywk |
| 781 | nrtqeqcvhn etknelekml kcneehpayl asdeittvrk nlesrgvevd pslikdtwhq |
| 841 | vyrrhflkta lnhcnlcrrg fyyyqrhfvd selecndvvl fwriqrmlai tantlrqqlt |
| 901 | ntevrrlekn vkevledfae dgekkikllt gkrvqlaedl kkvreiqekl dafiealhqe |
| 961 | k |

| SEQ ID No. 11: | |
|---|---|
| Nucleotide sequence encoding OPA1 spliceform 3 | |
| (NM_130832; CDS 56-2884) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttggtcacaa attggttagt gaagtcatag gagcttctga |
| 541 | cctacttctc ttgttaggtt ctccggaaga aacggcgttt agagcaacag atcgtggatc |
| 601 | tgaaagtgac aagcatttta gaaaggtgtc agacaaagag aaaattgacc aacttcagga |
| 661 | agaacttctg cacactcagt tgaagtatca gagaatcttg gaacgattag aaaaggagaa |
| 721 | caaagaattg agaaaattag tattgcagaa agatgacaaa ggcattcatc atagaaagct |
| 781 | taagaaatct ttgattgaca tgtattctga agttcttgat gttctctctg attatgatgc |
| 841 | cagttataat acgcaagatc atctgccacg ggttgttgtg gttggagatc agagtgctgg |
| 901 | aaagactagt gtgttggaaa tgattgccca agctcgaata ttcccaagag gatctgggga |
| 961 | gatgatgaca cgttctccag ttaaggtgac tctgagtgaa ggtcctcacc atgtggccct |
| 1021 | atttaaagat agttctcggg agtttgatct taccaaagaa gaagatcttg cagcattaag |
| 1081 | acatgaaata gaacttcgaa tgaggaaaaa tgtgaaagaa ggctgtaccg ttagccctga |
| 1141 | gaccatatcc ttaaatgtaa aaggccctgg actacagagg atggtgcttg ttgacttacc |
| 1201 | aggtgtgatt aatactgtga catcaggcat ggctcctgac acaaaggaaa ctattttcag |
| 1261 | tatcagcaaa gcttacatgc agaatcctaa tgccatcata ctgtgtattc aagatggatc |
| 1321 | tgtggatgct gaacgcagta ttgttacaga cttggtcagt caaatggacc ctcatggaag |
| 1381 | gagaaccata ttcgttttga ccaaagtaga cctggcagag aaaaatgtag ccagtccaag |
| 1441 | caggattcag cagataattg aaggaaagct cttcccaatg aaagctttag gttattttgc |
| 1501 | tgttgtaaca ggaaaaggga acagctctga aagcattgaa gctataagag aatatgaaga |
| 1561 | agagtttttt cagaattcaa agctcctaaa gacaagcatg ctaaaggcac accaagtgac |
| 1621 | tacaagaaat ttaagccttg cagtatcaga ctgcttttgg aaaatggtac gagagtctgt |
| 1681 | tgaacaacag gctgatagtt tcaaagcaac acgttttaac cttgaaactg aatggaagaa |
| 1741 | taactatcct cgcctgcggg aacttgaccg gaatgaacta tttgaaaaag ctaaaaatga |
| 1801 | aatccttgat gaagttatca gtctgagcca ggttacacca aaacattggg aggaaatcct |
| 1861 | tcaacaatct ttgtgggaaa gagtatcaac tcatgtgatt gaaaacatct accttccagc |
| 1921 | tgcgcagacc atgaattcag gaacttttaa caccacagtg gatatcaagc ttaaacagtg |
| 1981 | gactgataaa caacttccta ataaagcagt agaggttgct tgggagaccc tacaagaaga |
| 2041 | attttcccgc tttatgacag aaccgaaagg gaaagagcat gatgacatat ttgataaact |
| 2101 | taaagaggcc gttaaggaag aaagtattaa acgacacaag tggaatgact ttgcggagga |
| 2161 | cagcttgagg gttattcaac acaatgcttt ggaagaccga tccatatctg ataaacagca |
| 2221 | atgggatgca gctatttatt ttatggaaga ggctctgcag gctcgtctca aggatactga |
| 2281 | aaatgcaatt gaaaacatgg tgggtccaga ctggaaaaag aggtggttat actggaagaa |
| 2341 | tcggacccaa gaacagtgtg ttcacaatga aaccaagaat gaattggaga agatgttgaa |
| 2401 | atgtaatgag gagcacccag cttatcttgc aagtgatgaa ataaccacag tccggaagaa |
| 2461 | ccttgaatcc cgaggagtag aagtagatcc aagcttgatt aaggatactt ggcatcaagt |
| 2521 | ttatagaaga cattttttaa aaacagctct aaaccattgt aacctttgtc gaagaggttt |
| 2581 | ttattactac caaaggcatt ttgtagattc tgagttggaa tgcaatgatg tggtcttgtt |
| 2641 | ttggcgtata cagcgcatgc ttgctatcac cgcaaatact ttaaggcaac aacttacaaa |
| 2701 | tactgaagtt aggcgattag agaaaaatgt taaagaggta ttggaagatt ttgctgaaga |
| 2761 | tggtgagaag aagattaaat tgcttactgg taaacgcgtt caactggcgg aagacctcaa |
| 2821 | gaaagttaga gaaattcaag aaaaacttga tgctttcatt gaagctcttc atcaggagaa |
| 2881 | ataa |

| SEQ ID No. 12: | |
|---|---|
| Amino acid sequence of OPA1 spliceform 3 (NP_570845; 942 aa) | |
| 1 | mwrlrraava cevcqslvkh ssgikgslpl qklhlvsrsi yhshhptlkl qrpqlrtsfq |
| 61 | qfssltnlpl rklkfspiky gyqprrnfwp arlatrllkl rylilgsavg ggytakktfd |
| 121 | qwkdmipdls eykwivpdiv weideyidfg hklvseviga sdlllllgsp eetafratdr |
| 181 | gsesdkhfrk vsdkekidql qeellhtqlk yqrilerlek enkelrklvl qkddkgihhr |
| 241 | klkkslidmy sevldvlsdy dasyntqdhl prvvvvgdqs agktsvlemi aqarifprgs |
| 301 | gemmtrspvk vtlsegphhv alfkdssref dltkeedlaa lrheielrmr knvkegctvs |
| 361 | petislnvkg pglqrmvlvd lpgvintvts gmapdtketi fsiskaymqn pnaiilciqd |
| 421 | gsvdaersiv tdlvsqmdph grrtifvltk vdlaeknvas psriqqiieg klfpmkalgy |
| 481 | favvtgkgns sesieairey eeeffqnskl lktsmlkahq vttrnlslav sdcfwkmvre |
| 541 | sveqqadsfk atrfnletew knnyprlrel drnelfekak neildevisl sqvtpkhwee |
| 601 | ilqqslwerv sthvieniyl paaqtmnsgt fnttvdiklk qwtdkqlpnk avevawetlq |
| 661 | eefsrfmtep kgkehddifd klkeavkees ikrhkwndfa edslrviqhn aledrsisdk |
| 721 | qqwdaaiyfm eealqarlkd tenaienmvg pdwkkrwlyw knrtqeqcvh netknelekm |
| 781 | lkcneehpay lasdeittvr knlesrgvev dpslikdtwh qvyrrhflkt alnhcnlcrr |
| 841 | gfyyyqrhfv dselecndvv lfwriqrmla itantlrqql tntevrrlek nvkevledfa |
| 901 | edgekkikll tgkrvqlaed lkkvreiqek ldafiealhq ek |
| SEQ ID No. 13: | |
| Nucleotide sequence encoding OPA1 spliceform 2 | |
| (NM_130831; CDS 56-2830) | |
| 56 | atgtg |
| 61 | gcgactacgt cgggccgctg tggcctgtga ggtctgccag tctttagtga aacacagctc |
| 121 | tggaataaaa ggaagtttac cactacaaaa actacatctg gtttcacgaa gcatttatca |
| 181 | ttcacatcat cctaccttaa agcttcaacg accccaatta aggacatcct ttcagcagtt |
| 241 | ctcttctctg acaaaccttc ctttacgtaa actgaaattc tctccaatta aatatggcta |
| 301 | ccagcctcgc aggaattttt ggccagcaag attagctacg agactcttaa aacttcgcta |
| 361 | tctcatacta ggatcggctg ttgggggtgg ctacacagcc aaaaagactt ttgatcagtg |
| 421 | gaaagatatg ataccggacc ttagtgaata taaatggatt gtgcctgaca ttgtgtggga |
| 481 | aattgatgag tatatcgatt ttggttctcc ggaagaaacg gcgtttagag caacagatcg |
| 541 | tggatctgaa agtgacaagc attttagaaa ggtgtcagac aaagagaaaa ttgaccaact |
| 601 | tcaggaagaa cttctgcaca ctcagttgaa gtatcagaga atcttggaac gattagaaaa |
| 661 | ggagaacaaa gaattgagaa aattagtatt gcagaaagat gacaaaggca ttcatcatag |
| 721 | aaagcttaag aaatctttga ttgacatgta ttctgaagtt cttgatgttc tctctgatta |
| 781 | tgatgccagt tataatacgc aagatcatct gccacgggtt gttgtggttg gagatcagag |
| 841 | tgctggaaag actagtgtgt tggaaatgat tgcccaagct cgaatattcc caagaggatc |
| 901 | tggggagatg atgacacgtt ctccagttaa ggtgactctg agtgaaggtc ctcaccatgt |
| 961 | ggccctattt aaagatagtt ctcgggagtt tgatcttacc aaagaagaag atcttgcagc |
| 1021 | attaagacat gaaatagaac ttcgaatgag gaaaaatgtg aaagaaggct gtaccgttag |
| 1081 | ccctgagacc atatccttaa atgtaaaagg ccctggacta cagaggatgg tgcttgttga |
| 1141 | cttaccaggt gtgattaata ctgtgacatc aggcatggct cctgacacaa aggaaactat |
| 1201 | tttcagtatc agcaaagctt acatgcagaa tcctaatgcc atcatactgt gtattcaaga |
| 1261 | tggatctgtg gatgctgaac gcagtattgt tacagacttg gtcagtcaaa tggaccctca |
| 1321 | tggaaggaga accatattcg ttttgaccaa agtagacctg gcagagaaaa atgtagccag |
| 1381 | tccaagcagg attcagcaga taattgaagg aaagctcttc ccaatgaaag ctttaggtta |
| 1441 | ttttgctgtt gtaacaggaa aagggaacag ctctgaaagc attgaagcta taagagaata |
| 1501 | tgaagaagag ttttttcaga attcaaagct cctaaagaca agcatgctaa aggcacacca |
| 1561 | agtgactaca agaaatttaa gccttgcagt atcagactgc ttttggaaaa tggtacgaga |
| 1621 | gtctgttgaa caacaggctg atagtttcaa agcaacacgt tttaaccttg aaactgaatg |
| 1681 | gaagaataac tatcctcgcc tgcgggaact tgaccggaat gaactatttg aaaaagctaa |
| 1741 | aaatgaaatc cttgatgaag ttatcagtct gagccaggtt acaccaaaac attgggagga |
| 1801 | aatccttcaa caatctttgt gggaaagagt atcaactcat gtgattgaaa acatctacct |
| 1861 | tccagctgcg cagaccatga attcaggaac ttttaacacc acagtggata tcaagcttaa |
| 1921 | acagtggact gataaacaac ttcctaataa agcagtagag gttgcttggg agaccctaca |
| 1981 | agaagaattt tcccgcttta tgacagaacc gaaagggaaa gagcatgatg acatatttga |
| 2041 | taaacttaaa gaggccgtta aggaagaaag tattaaacga cacaagtgga atgactttgc |
| 2101 | ggaggacagc ttgagggtta ttcaacacaa tgctttggaa gaccgatcca tatctgataa |
| 2161 | acagcaatgg gatgcagcta tttattttat ggaagaggct ctgcaggctc gtctcaagga |
| 2221 | tactgaaaat gcaattgaaa acatggtggg tccagactgg aaaaagaggt ggttatactg |
| 2281 | gaagaatcgg acccaagaac agtgtgttca caatgaaacc aagaatgaat tggagaagat |
| 2341 | gttgaaatgt aatgaggagc acccagctta tcttgcaagt gatgaaataa ccacagtccg |
| 2401 | gaagaacctt gaatcccgag gagtagaagt agatccaagc ttgattaagg atacttggca |
| 2461 | tcaagtttat agaagacatt ttttaaaaac agctctaaac cattgtaacc tttgtcgaag |
| 2521 | aggtttttat tactaccaaa ggcattttgt agattctgag ttggaatgca atgatgtggt |
| 2581 | cttgttttgg cgtatacagc gcatgcttgc tatcaccgca aatactttaa ggcaacaact |
| 2641 | tacaaatact gaagttaggc gattagagaa aaatgttaaa gaggtattgg aagattttgc |
| 2701 | tgaagatggt gagaagaaga ttaaattgct tactggtaaa cgcgttcaac tggcggaaga |
| 2761 | cctcaagaaa gttagagaaa ttcaagaaaa acttgatgct ttcattgaag ctcttcatca |
| 2821 | ggagaaataa |
| SEQ ID No. 14: | |
| Amino acid sequence of OPA1 spliceform 2 (NP_570844; 924 aa) | |
| 1 | mwrlrraava cevcqslvkh ssgikgslpl qklhlvsrsi yhshhptlkl qrpqlrtsfg |
| 61 | qfssltnlpl rklkfspiky gyqprrnfwp arlatrllkl rylilgsavg ggytakktfd |
| 121 | qwkdmipdls eykwivpdiv weideyidfg speetafrat drgsesdkhf rkvsdkekid |
| 181 | qlqeellhtg lkygrilerl ekenkelrkl vlqkddkgih hrklkkslid mysevldvls |
| 241 | dydasyntqd hlprvvvvgd gsagktsvle miagarifpr gsgemmtrsp vkvtlsegph |
| 301 | hvalfkdssr efdltkeedl aalrheielr mrknvkegct vspetislnv kgpglqrmvl |
| 361 | vdlpgvintv tsgmapdtke tifsiskaym qnpnaiilci qdgsvdaers ivtdlvsqmd |
| 421 | phgrrtifvl tkvdlaeknv aspsriqqii egklfpmkal gyfavvtgkg nssesieair |
| 481 | eyeeeffqns kllktsmlka hqvttrnlsl avsdcfwkmv resveqqads fkatrfnlet |
| 541 | ewknnyprlr eldrnelfek akneildevi slsqvtpkhw eeilqqslwe rvsthvieni |
| 601 | ylpaaqtmns gtfnttvdik lkqwtdkqlp nkavevawet lqeefsrfmt epkgkehddi |
| 661 | fdklkeavke esikrhkwnd faedslrviq hnaledrsis dkqqwdaaiy fmeealqarl |
| 721 | kdtenaienm vgpdwkkrwl ywknrtqeqc vhnetknele kmlkcneehp aylasdeitt |
| 781 | vrknlesrgv evdpslikdt whqvyrrhfl ktalnhcnlc rrgfyyyqrh fvdselecnd |
| 841 | vvlfwriqrm laitantlrq qltntevrrl eknvkevled faedgekkik lltgkrvqla |
| 901 | edlkkvreiq ekldafieal hqek |
| SEQ ID No. 15: | |
| Nucleotide sequence encoding OPA1 spliceform 1 | |
| (BC075805; CDS 155-3037) | |
| 155 | atgtgg cgactacgtc gggccgctgt |
| 181 | ggcctgtgag gtctgccagt ctttagtgaa acacagctct ggaataaaag gaagtttacc |
| 241 | actacaaaaa ctacatctgg tttcacgaag catttatcat tcacatcatc ctaccttaaa |
| 301 | gcttcaacga ccccaattaa ggacatcctt tcagcagttc tcttctctga caaaccttcc |
| 361 | tttacgtaaa ctgaaattct ctccaattaa atatggctac cagcctcgca ggaatttttg |
| 421 | gccagcaaga ttagctacga gactcttaaa acttcgctat ctcatactag gatcggctgt |
| 481 | tgggggtggc tacacagcca aaaagacttt tgatcagtgg aaagatatga taccggacct |
| 541 | tagtgaatat aaatggattg tgcctgacat tgtgtgggaa attgatgagt atatcgattt |
| 601 | tgagaaaatt agaaaagccc ttcctaattc agaagacctt gtaaagttag caccagactt |
| 661 | tgacaagatt gttgaaagcc ttagcttatt gaaggacttt tttacctcag gttctccgga |
| 721 | agaaacggcg tttagagcaa cagatcgtgg atctgaaagt gacaagcatt ttagaaaggt |
| 781 | gtcagacaaa gagaaaattg accaacttca ggaagaactt ctgcacactc agttgaagta |
| 841 | tcagagaatc ttggaacgat tagaaaagga gaacaaagaa ttgagaaaat tagtattgca |
| 901 | gaaagatgac aaaggcattc atcatagaaa gcttaagaaa tctttgattg acatgtattc |
| 961 | tgaagttctt gatgttctct ctgattatga tgccagttat aatacgcaag atcatctgcc |
| 1021 | acgggttgtt gtggttggag atcagagtgc tggaaagact agtgtgttgg aaatgattgc |
| 1081 | ccaagctcga atattcccaa gaggatctgg ggagatgatg acacgttctc cagttaaggt |
| 1141 | gactctgagt gaaggtcctc accatgtggc cctatttaaa gatagttctc gggagtttga |
| 1201 | tcttaccaaa gaagaagatc ttgcagcatt aagacatgaa atagaacttc gaatgaggaa |
| 1261 | aaatgtgaaa gaaggctgta ccgttagccc tgagaccata tccttaaatg taaaaggccc |
| 1321 | tggactacag aggatggtgc ttgttgactt accaggtgtg attaatactg tgacatcagg |
| 1381 | catggctcct gacacaaagg aaactatttt cagtatcagc aaagcttaca tgcagaatcc |
| 1441 | taatgccatc atactgtgta ttcaagatgg atctgtggat gctgaacgca gtattgttac |
| 1501 | agacttggtc agtcaaatgg accctcatgg aaggagaacc atattcgttt tgaccaaagt |
| 1561 | agacctggca gagaaaaatg tagccagtcc aagcaggatt cagcagataa ttgaaggaaa |
| 1621 | gctcttccca atgaaagctt taggttattt tgctgttgta acaggaaaag ggaacagctc |
| 1681 | tgaaagcatt gaagctataa gagaatatga agaagagttt tttcagaatt caaagctcct |
| 1741 | aaagacaagc atgctaaagg cacaccaagt gactacaaga aatttaagcc ttgcagtatc |
| 1801 | agactgcttt tggaaaatgg tacgagagtc tgttgaacaa caggctgata gtttcaaagc |
| 1861 | aacacgtttt aaccttgaaa ctgaatggaa gaataactat cctcgcctgc gggaacttga |
| 1921 | ccggaatgaa ctatttgaaa aagctaaaaa tgaaatcctt gatgaagtta tcagtctgag |
| 1981 | ccaggttaca ccaaaacatt gggaggaaat ccttcaacaa tctttgtggg aaagagtatc |
| 2041 | aactcatgtg attgaaaaca tctaccttcc agctgcgcag accatgaatt caggaacttt |
| 2101 | taacaccaca gtggatatca agcttaaaca gtggactgat aaacaacttc ctaataaagc |
| 2161 | agtagaggtt gcttgggaga ccctacaaga agaattttcc cgctttatga cagaaccgaa |
| 2221 | agggaaagag catgatgaca tatttgataa acttaaagag gccgttaagg aagaaagtat |
| 2281 | taaacgacac aagtggaatg actttgcgga ggacagcttg agggttattc aacacaatgc |
| 2341 | tttggaagac cgatccatat ctgataaaca gcaatgggat gcagctattt attttatgga |
| 2401 | agaggctctg caggctcgtc tcaaggatac tgaaaatgca attgaaaaca tggtgggtcc |
| 2461 | agactggaaa aagaggtggt tatactggaa gaatcggacc caagaacagt gtgttcacaa |
| 2521 | tgaaaccaag aatgaattgg agaagatgtt gaaatgtaat gaggagcacc cagcttatct |
| 2581 | tgcaagtgat gaaataacca cagtccggaa gaaccttgaa tcccgaggag tagaagtaga |
| 2641 | tccaagcttg attaaggata cttggcatca agtttataga agacattttt taaaaacagc |
| 2701 | tctaaaccat tgtaaccttt gtcgaagagg tttttattac taccaaaggc attttgtaga |
| 2761 | ttctgagttg gaatgcaatg atgtggtctt gttttggcgt atacagcgca tgcttgctat |
| 2821 | caccgcaaat actttaaggc aacaacttac aaatactgaa gttaggcgat tagagaaaaa |
| 2881 | tgttaaagag gtattggaag attttgctga agatggtgag aagaagatta aattgcttac |
| 2941 | tggtaaacgc gttcaactgg cggaagacct caagaaagtt agagaaattc aagaaaaact |
| 3001 | tgatgctttc attgaagctc ttcatcagga gaaataa |
| SEQ ID No. 16: | |
| Amino acid sequence of OPA1 spliceform 1 (AAH75805; 960 aa) | |
| 1 | mwrlrraava cevcqslvkh ssgikgslpl qklhlvsrsi yhshhptlkl qrpqlrtsfg |
| 61 | qfssltnlpl rklkfspiky qyqprrnfwp arlatrllkl rylilgsavg ggytakktfd |
| 121 | qwkdmipdls eykwivpdiv weideyidfe kirkalpnse dlvklapdfd kiveslsllk |
| 181 | dfftsgspee tafratdrgs esdkhfrkvs dkekidqlqe ellhtqlkyq rilerleken |
| 241 | kelrklvlqk ddkgihhrkl kkslidmyse vldvlsdyda syntqdhlpr vvvvgdqsag |
| 301 | ktsvlemiaq arifprgsge mmtrspvkvt lsegphhval fkdssrefdl tkeedlaalr |
| 361 | heielrmrkn vkegctvspe tislnvkgpg lgrmvlvdlp gvintvtsgm apdtketifs |
| 421 | iskaymqnpn aiilciqdgs vdaersivtd lvsqmdphgr rtifvltkvd laeknvasps |
| 481 | riqqiiegkl fpmkalgyfa vvtgkgnsse sieaireyee effqnskllk tsmlkahqvt |
| 541 | trnlslavsd cfwkmvresv eqqadsfkat rfnletewkn nyprlreldr nelfekakne |
| 601 | ildevislsq vtpkhweeil qqslwervst hvieniylpa aqtmnsgtfn ttvdiklkqw |
| 661 | tdkqlpnkav evawetlqee fsrfmtepkg kehddifdkl keavkeesik rhkwndfaed |
| 721 | slrviqhnal edrsisdkqq wdaaiyfmee alqarlkdte naienmvgpd wkkrwlywkn |
| 781 | rtqeqcvhne tknelekmlk cneehpayla sdeittvrkn lesrgvevdp slikdtwhqv |
| 841 | yrrhflktal nhcnlcrrgf yyyqrhfvds elecndvvlf wriqrmlait antlrqqltn |
| 901 | tevrrleknv kevledfaed gekkiklltg krvqlaedlk kvreiqekld afiealhqek |
| SEQ ID No. 37: | |
| Nucleotide sequence encoding Afg3I1 from mouse | |
| (NM_054070; CDS 8-2377) | |
| 8 | atg ttactgcggc tggtgggggc ggcgggcagt cgagccctgg cctggccttt |
| 61 | ctccaagctg tggcgatgtg gcggatgcgc agggagcggc gggacggtct ggagcagcgt |
| 121 | gagggcctgt ggcattgctc tgcagggtca tctggggaga tgctcgcagc agctggctct |
| 181 | gcagggaaaa ctgacttcat tttccccgag gctgtattca aaacctccca gagggtttga |
| 241 | gaagtttttt aagaataaga agaacagaaa aagtgcaagc ccaggaaatt cagtacctcc |
| 301 | aaaaaaagaa ccaaaaaatg ctggccctgg aggagatgga ggcaacagag gagggaaagg |
| 361 | agatgatttt ccctggtgga aacggatgca aaagggagaa tttccttggg acgacaagga |
| 421 | cttccggagc ctggctgttt tgggggctgg tgtggctgcg ggatttttat atttttattt |
| 481 | ccgagatccc ggaaaagaga tcacctggaa acacttcgtg cagtattacc tggccagagg |
| 541 | tctggtggac cggctagagg ttgtgaacaa gcagtttgtg cgtgttattc ctgttcctgg |
| 601 | gacgacatct gagaggttcg tgtggtttaa cattggcagt gttgacacct ttgaacggaa |
| 661 | cctcgagtct gctcagtggg agctgggcat tgagcccacc aaccaggctg cggtggtcta |
| 721 | cactactgag agtgatggct cttttcttag aagtcttgtg cccactctgg tcctggttag |
| 781 | catcctccta tatgctatga ggaggggtcc aatggggact ggtcgcggtg ggcgaggagg |
| 841 | aggcctcttc agtgttggtg agacaacagc caagatctta aagaacaaca tcgatgtgcg |
| 901 | gtttgcagat gtggctggct gtgaagaagc caaactggaa attatggagt ttgtgaattt |
| 961 | cctgaagaac ccaaagcaat atcaggactt aggagccaaa attccaaagg gagcgatgct |
| 1021 | cactggtcca cctggtactg gcaaaacact tcttgcaaaa gcaactgctg gggaggccaa |
| 1081 | cgtgcccttc atcaccgtga atgggtcgga attcctggaa atgtttgttg gtgttgggcc |
| 1141 | agcacgggtc cgtgacatgt ttgcaatggc ccgaaaacac gctccttgta ttttattcat |
| 1201 | tgatgagatt gatgcaattg gcagaaagcg aggccgtggc cacctgggag gccagagtga |
| 1261 | gcaggaaaac actttaaacc agatgcttgt ggagatggac gggttcaact cttccactaa |
| 1321 | tgtggtagtg ttagccggca ccaatcgccc tgatatcctt gacccagccc tgacacggcc |
| 1381 | tggccggttt gaccgtcaga tctacatcgg tccccctgat atcaaaggca ggtcctctat |
| 1441 | tttcaaggtc cacttgcgtc cactcaagct ggacggaagc ctcagcaagg acgctctttc |
| 1501 | gaggaagctg gcagctctta ctccaggctt cactggtgct gatatttcca atgtttgcaa |
| 1561 | tgaagcagca ctgattgctg cccgccacct cagcccttct gtccaggagc ggcactttga |
| 1621 | gcaagccatc gagagggtca ttggaggcct tgagaagaag acccaggtcc tacaacccag |
| 1681 | tgaaaagaca actgtagcct accacgaggc tgggcatgca gtggtgggct ggttcttgga |
| 1741 | gcatgcagac cctctgctga aggtgtccat catacctcga ggcaaggggc ttggctacgc |
| 1801 | ccagtacctt ccccgcgagc agttcctcta cacacgagag cagctcttcg accgcatgtg |
| 1861 | tatgatgctg gggggtaggg tagctgagca gctgttcttt ggtcagatca ccaccggagc |
| 1921 | tcaggacgac ctgaggaagg tcacccagag tgcctatgcc cagattgtgc agtttgggat |
| 1981 | gagtgagaag ctgggccagg tgtcctttga cttccccaga caaggcgaaa ccatggtgga |
| 2041 | gaagccatac agtgaggcta ctgcccagct cattgatgaa gaggtccggt gcctcgtcag |
| 2101 | gtctgcctat aatcggaccc tggagctgct cacacagtgc cgggagcagg tggagaaggt |
| 2161 | tggcaggcgt ctcctggaga aagaagtgct ggagaaagcc gacatgatag agctcttggg |
| 2221 | ccctcggccc tttgcagaga agtccaccta tgaggaattt gtagagggca ccggcagcct |
| 2281 | agaggaggac acatcccttc ctgaggggct gaaagactgg aataaggggc gggaggaagg |
| 2341 | aggcactgag cggggcttgc aggagagccc tgtgtag |
| SEQ ID No. 38: | |
| Amino acid sequence of Afg3I1 from mouse (NP_473411; 789 aa) | |
| 1 | mllrlvgaag sralawpfsk lwrcggcags ggtvwssvra cgialgghlg rcsqqlalqg |
| 61 | kltsfsprly skpprgfekf fknkknrksa spgnsvppkk epknagpggd ggnrggkgdd |
| 121 | fpwwkrmqkg efpwddkdfr slavlgagva agflyfyfrd pgkeitwkhf vqyylarglv |
| 181 | drlevvnkqf vrvipvpgtt serfvwfnig svdtfernle saqwelgiep tnqaavvytt |
| 241 | esdgsflrsl vptlvlvsil lyamrrgpmg tgrggrgggl fsvgettaki lknnidvrfa |
| 301 | dvagceeakl eimefvnflk npkqyqdlga kipkgamltg ppgtgktlla katageanvp |
| 361 | fitvngsefl emfvgvgpar vrdmfamark hapcilfide idaigrkrgr ghlggqseqe |
| 421 | ntlnqmlvem dgfnsstnvv vlagtnrpdi ldpaltrpgr fdrgiyigpp dikgrssifk |
| 481 | vhlrplkldg slskdalsrk laaltpgftg adisnvcnea aliaarhlsp svqerhfeqa |
| 541 | iervigglek ktqvlqpsek ttvayheagh avvgwfleha dpllkvsiip rgkglgyaqy |
| 601 | lpreqflytr eqlfdrmcmm lggrvaeqlf fgqittgaqd dlrkvtqsay aqivqfgmse |
| 661 | klgqvsfdfp rggetmvekp yseataqlid eevrclvrsa ynrtlelltq creqvekvgr |
| 721 | rllekevlek admiellgpr pfaekstyee fvegtgslee dtslpeglkd wnkgreeggt |
| 781 | erglqespv |
| SEQ ID No. 39: | |
| Nucleotide sequence encoding Afg3I2 from human | |
| (NM_006796; CDS114-2507) | |
| 114 | atggcgc |
| 121 | accgctgttt gcggctgtgg ggccggggcg gctgctggcc ccgcggccta cagcagctcc |
| 181 | tcgtgcctgg cggcgtgggc ccgggcgagc agccctgcct ccggacgctt taccgatttg |
| 241 | ttacaactca agcaagggcc agcagaaatt ctcttttgac agatataatt gctgcttatc |
| 301 | aaagattctg ttctcgaccc ccaaaaggat ttggaaaata ctttcctaat ggaaaaaatg |
| 361 | gaaaaaaagc tagtgaacct aaagaagtta tgggagagaa aaaagaatca aagccagctg |
| 421 | ctaccacacg ctcttctgga ggaggaggtg gtggcggtgg aaaacgaggt ggcaagaaag |
| 481 | atgattctca ctggtggtcc aggtttcaga agggtgacat tccatgggac gacaaggatt |
| 541 | tcaggatgtt cttcctctgg actgctctgt tctggggtgg agtcatgttt tacttgctgc |
| 601 | tcaagagatc cgggagagaa atcacttgga aggactttgt caataactat ctttcaaaag |
| 661 | gagtagtaga cagattggaa gtcgtcaaca agcgttttgt tcgagtgacc tttacaccag |
| 721 | gaaaaactcc tgttgatggg caatacgttt ggtttaatat tggcagtgtg gacacctttg |
| 781 | aacggaatct ggaaacttta cagcaggaat tgggcataga aggagaaaat cgggtgcctg |
| 841 | ttgtctacat tgctgaaagt gatggctctt ttctgctgag catgctgcct acggtgctca |
| 901 | tcatcgcctt cttgctctac accatcagaa gagggcctgc tggcattggc cggacaggcc |
| 961 | gagggatggg cggactcttc agtgtcggag aaaccactgc caaggtctta aaggatgaaa |
| 1021 | ttgatgtgaa gttcaaagat gtggctggct gtgaggaggc caagctagag atcatggaat |
| 1081 | ttgtgaattt cttgaaaaac ccaaagcagt atcaagacct aggagcaaaa atcccaaagg |
| 1141 | gtgccattct cactggtcct ccaggcactg ggaagacgct gctagctaag gccacagccg |
| 1201 | gagaagccaa tgtccccttc atcaccgtta gtggatctga gtttttggag atgttcgttg |
| 1261 | gtgtgggccc tgctagagtc cgagacttat ttgcccttgc tcggaagaat gccccttgca |
| 1321 | tcctcttcat cgatgaaatc gatgcggtgg gaaggaagag aggaagaggc aactttggag |
| 1381 | ggcagagtga gcaggagaac acactcaacc agctgctggt ggagatggat ggttttaata |
| 1441 | caacaacaaa tgtcgtcatt ttggccggca ccaatcgacc agatatcctg gaccccgcgc |
| 1501 | tacttaggcc ggggcgtttc gacaggcaga tctttattgg accaccagac ataaaaggaa |
| 1561 | gagcttctat tttcaaagtt catctccgac cgctaaaact ggacagtacc ctggagaagg |
| 1621 | ataaattggc aagaaaactg gcatctttaa ctccagggtt ttcaggtgct gatgttgcta |
| 1681 | atgtctgtaa tgaagctgcg ttgattgctg caaggcatct gtcagattcc ataaatcaga |
| 1741 | aacactttga acaggcaatt gagcgagtga ttggtggctt agagaagaaa acgcaggttc |
| 1801 | tgcagcctga ggagaagaag actgtggcat accacgaagc aggccatgcg gttgccggct |
| 1861 | ggtatctgga gcacgcagac ccgcttttaa aggtatccat catcccacgt ggcaaaggac |
| 1921 | taggttatgc tcagtattta ccaaaagaac aatacctcta taccaaagag cagctcttgg |
| 1981 | ataggatgtg tatgacttta ggtggtcgag cctctgaaga aatcttcttt ggaagaatta |
| 2041 | caactggtgc tcaagatgac ttgagaaaag taactcagag tgcatatgcc caaattgttc |
| 2101 | agtttggcat gaatgaaaag gttgggcaaa tctcctttga cctcccacgt cagggggaca |
| 2161 | tggtattgga gaaaccttac agtgaagcca ctgcaagatt gatagatgat gaagtacgaa |
| 2221 | tacttattaa tgatgcttat aaaagaacag tagctcttct cacagaaaag aaagctgacg |
| 2281 | tggagaaggt tgctcttctg ttgttagaaa aagaagtatt agataagaat gatatggttg |
| 2341 | aacttttggg ccccagacca tttgcggaaa aatctaccta tgaagaattt gtggaaggca |
| 2401 | ctggcagctt ggatgaggac acctcacttc cagaaggcct taaggactgg aacaaggagc |
| 2461 | gggaaaagga gaaagaggag cccccgggtg agaaagttgc caactag |
| SEQ ID No. 40: | |
| Amino acid sequence of Afg3I2 from human (NP_006787; 797 aa) | |
| 1 | mahrclrlwg rggcwprglg gllvpggvgp geqpclrtly rfvttqaras rnslltdiia |
| 61 | ayqrfcsrpp kgfgkyfpng kngkkasepk evmgekkesk paattrssgg ggggggkrgg |
| 121 | kkddshwwsr fqkgdipwdd kdfrmfflwt alfwggvmfy lllkrsgrei twkdfvnnyl |
| 181 | skgvvdrlev vnkrfvrvtf tpgktpvdgq yvwfnigsvd tfernletlq qelgiegenr |
| 241 | vpvvyiaesd gsfllsmlpt vliiafllyt irrgpagigr tgrgmgglfs vgettakvlk |
| 301 | deidvkfkdv agceeaklei mefvnflknp kqyqdlgaki pkgailtgpp gtgktllaka |
| 361 | tageanvpfi tvsgseflem fvgvgparvr dlfalarkna pcilfideid avgrkrgrgn |
| 421 | fggqseqent lnqllvemdg fntttnvvil agtnrpdild pallrpgrfd rqifigppdi |
| 481 | kgrasifkvh lrplkldstl ekdklarkla sltpgfsgad vanvcneaal iaarhlsdsi |
| 541 | nqkhfeqaie rvigglekkt qvlqpeekkt vayheaghav agwylehadp llkvsiiprg |
| 601 | kglgyaqylp kegylytkeq lldrmcmtlg graseeiffg rittgaqddl rkvtqsayaq |
| 661 | ivqfgmnekv gqisfdlprq gdmvlekpys eatarlidde vrilindayk rtvalltekk |
| 721 | advekvalll lekevldknd mvellgprpf aekstyeefv egtgsldedt slpeglkdwn |
| 781 | kerekekeep pgekvan |
| SEQ ID No. 41: | |
| Nucleotide sequence encoding Afg3I2 from mouse | |
| (NM_027130; CDS 136-2544) | |
| 136 | atggc ccaccgctgc ctgctgctgt ggagccgggg cggctgccgt |
| 181 | cgcggccttc ctcccctgct cgtgcccaga ggttgcctgg gtccggaccg gcggccctgc |
| 241 | ctccgtacgc tctatcaata tgctactgtc cagacagcaa gcagcaggcg ttctctgctg |
| 301 | agggatgtaa ttgctgctta tcaaagattc tgttctcgac ctcccaaagg atttgaaaag |
| 361 | tactttccta atgggaaaaa cggaaaaaag gccagtgagc ctaaggaggc tgttggagaa |
| 421 | aaaaaagaac cacagccctc gggcccccag ccttctggag gtgcaggtgg tgggggaggg |
| 481 | aagcgccgtg gcaagaaaga agattctcac tggtggtcca ggttccagaa gggtgacttc |
| 541 | ccatgggatg acaaggattt caggatgtac tttctctgga ctgctctttt ttggggtgga |
| 601 | gtcatgattt acttcgtgtt caagagctct gggagagaaa tcacgtggaa agactttgtc |
| 661 | aataactatc tttctaaggg cgtggtggac agactagaag ttgtcaacaa gcgttttgtt |
| 721 | cgtgtgacct ttacaccagg aaaaactccg gttgatgggc aatacgtctg gtttaatatt |
| 781 | ggcagtgttg acacatttga gcggaatctg gagactttgc agcaagaatt gggcatagaa |
| 841 | ggggagaacc gggtccctgt ggtttatatt gctgagagcg atggctcctt cctgctgagc |
| 901 | atgttgccca ccgtactcat tatcgctttt ctactctaca ccataagaag agggcccgct |
| 961 | ggcattggtc ggaccggccg gggaatgggt ggactcttca gcgttgggga aaccacagcc |
| 1021 | aaggtcttaa aggatgagat agatgtgaag tttaaagatg tggctggctg tgaggaggcc |
| 1081 | aagctagaaa taatggaatt cgtgaatttc ttgaaaaacc caaagcaata tcaagaccta |
| 1141 | ggagcaaaaa tcccaaaggg tgccattctc accggtcccc caggtactgg gaagacgctg |
| 1201 | ctagctaagg ccacagctgg agaagccaat gtccccttta tcactgtgag cggatctgag |
| 1261 | tttctggaga tgtttgttgg cgttggtcca gccagagtcc gagacttatt tgcccttgct |
| 1321 | cggaagaatg cgccttgcat tctcttcatt gatgagattg atgctgtggg aaggaagcgc |
| 1381 | ggcagaggca acttcggtgg gcagagcgag caggagaaca cactcaacca gctgcttgtg |
| 1441 | gagatggacg gcttcaacac aaccaccaat gtggtcatcc tggcaggcac aaatcgacca |
| 1501 | gacatcctgg atccagctct gttgagacca ggccgctttg acaggcagat ttttattgga |
| 1561 | cccccagaca taaaaggacg agcctcaatc ttcaaagttc accttcgacc attgaagctg |
| 1621 | gacagtgcct tggaaaaaga taaattggcc agaaaactgg cgtccttaac tccagggttt |
| 1681 | tcaggcgctg atgttgccaa tgtctgcaat gaagctgctt tgattgctgc aagacacctt |
| 1741 | tcagatgcca ttaatgagaa gcacttcgaa caagcgattg agcgagtgat tggaggcttg |
| 1801 | gagaaaaaaa cccaagttct gcagcctgag gagaagaaga cggtggctta ccacgaagca |
| 1861 | ggccatgcgg tcgctggctg gtatctggag catgcagacc cactcttaaa ggtctccatc |
| 1921 | atcccgcgtg gcaaggggct gggctatgct cagtacttgc ccaaggagca gtatctgtac |
| 1981 | acaaaggagc agctgctgga caggatgtgc atgactctgg gcggccgtgt ctccgaggag |
| 2041 | atcttctttg ggagaattac aaccggtgcc caggacgact tgaggaaggt tacccagagt |
| 2101 | gcctatgccc agatcgttca gtttggcatg aacgagaaag tggggcagat ctcctttgac |
| 2161 | ctcccacgac agggggacat ggtgttagag aagccttaca gtgaggccac tgcgaggatg |
| 2221 | atagacgatg aagtgaggat actcatcagc gatgcctaca gaaggacggt ggctcttctc |
| 2281 | acagagaaga aggctgacgt ggagaaggtc gctctcttac tgttagaaaa ggaagtccta |
| 2341 | gacaagaatg acatggtcca gcttctcggt cccagaccat ttacagaaaa gtccacatat |
| 2401 | gaagaatttg tggaaggcac tggcagctta gacgaggaca cttctcttcc tgaaggcctt |
| 2461 | caggattgga acaaggagcg ggagaaggag gagaagaagg agaaggagaa ggaggagccg |
| 2521 | ctgaatgaga aggttgtcag ctag |
| SEQ ID No. 42: | |
| Amino acid sequence of Afg3I2 from mouse (NP_081406; 802 aa) | |
| 1 | mahrclllws rggcrrglpp llvprgclgp drrpclrtly qyatvqtass rrsllrdvia |
| 61 | ayqrfcsrpp kgfekyfpng kngkkasepk eavgekkepq psgpqpsgga gggggkrrgk |
| 121 | kedshwwsrf qkgdfpwddk dfrmyflwta lfwggvmiyf vfkssgreit wkdfvnnyls |
| 181 | kgvvdrlevv nkrfvrvtft pgktpvdgqy vwfnigsvdt fernletlqq elgiegenrv |
| 241 | pvvyiaesdg sfllsmlptv liiafllyti rrgpagigrt grgmgglfsv gettakvlkd |
| 301 | eidvkfkdva gceeakleim efvnflknpk qyqdlgakip kgailtgppg tgktllakat |
| 361 | ageanvpfit vsgseflemf vgvgparvrd lfalarknap cilfideida vgrkrgrgnf |
| 421 | ggqseqentl nqllvemdgf ntttnvvila gtnrpdildp allrpgrfdr qifigppdik |
| 481 | grasifkvhl rplkldsale kdklarklas ltpgfsgadv anvcneaali aarhlsdain |
| 541 | ekhfeqaier vigglekktq vlqpeekktv ayheaghava gwylehadpl lkvsiiprgk |
| 601 | glgyaqylpk eqylytkeql ldrmcmtlgg rvseeiffgr ittgagddlr kvtqsayagi |
| 661 | vqfgmnekvg qisfdlprqg dmvlekpyse atarmiddev rilisdayrr tvalltekka |
| 721 | dvekvallll ekevldkndm vqllgprpft ekstyeefve gtgsldedts lpeglqdwnk |
| 781 | erekeekkek ekeeplnekv vs |

1. (canceled)

2. A method for the treatment, prevention and/or amelioration of
(i) a disorder or disease correlated with mitochondrial dysfunction or a mitochondrial disorder or disease; or
(ii) a disorder or disease characterized by an altered OPA1 processing,
wherein said method comprises the administration to a patient in need of medical intervention a pharmaceutically active amount of a compound capable of modulating the activity of an oligomeric complex comprising Afg 3l1 and/or Afg 3l2 or (a) variant(s) thereof.

3. Method of screening for a compound capable of modulating the activity of an oligomeric complex comprising Afg 3l1 and/or Afg 3l2 or (a) variant(s) thereof comprising the steps of
(a) contacting OPA1 with said oligomeric complex comprising Afg 3l1 and/or Afg 3l2 or (a) variant(s) thereof in the presence of said compound to be screened for under conditions allowing OPA1 processing to occur; and
(b) evaluation whether OPA1 processing is altered compared to a control, where OPA1 and said oligomeric complex comprising Afg 3l1 and/or Afg 3l2 or (a) variant(s) thereof are contacted in the absence of said compound to be screened for under conditions allowing OPA 1 processing to occur.

4. The method of claim 2, wherein said oligomeric complex comprising Afg 3l1 and/or Afg 3l2 or (a) variant(s) thereof comprises a polypeptide selected from the group consisting of:
(a) a polypeptide comprising an amino acid sequence as depicted in SEQ ID NO 38, 40 or 42;
(b) a polypeptide comprising an amino acid sequence encoded by a nucleic acid molecule as depicted in SEQ ID NO 37, 39 or 41;
(c) a polypeptide comprising an amino acid sequence encoded by a nucleic acid molecule encoding an amino acid sequence as depicted in SEQ ID NO 38, 40 or 42;
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (b) to (c);
(f) a polypeptide comprising an amino acid sequence encoded by a nucleic acid molecule hybridizing under stringent conditions to the complement stand of a nucleic acid molecule as defined in any one of (b) to (c); and
(g) fragment of a polypeptide of any one of (b) to (f).

5. The method of claim 2, wherein said oligomeric complex comprising Afg 3l1 and/or Afg 3l2 or (a) variant(s) thereof is a homo-oligomeric complex or a hetero-oligomeric complex.

6. The method of claim 5, wherein said hetero-oligomeric complex comprises paraplegin or a variant thereof.

7. The method of claim 2, wherein said compound capable of modulating the activity of an oligomeric complex comprising Afg 3l1 and/or Afg 3l2 or (a) variant(s) thereof is a compound obtained by a method comprising the steps of
(a) contacting OPA1 with said oligomeric complex comprising Afg 3l1 and/or Afg 3l2 or (a) variant(s) thereof in the presence of said compound to be screened for under conditions allowing OPA1 processing to occur; and
(b) evaluation whether OPA1 processing is altered compared to a control, where OPA1 and said oligomeric complex comprising Afg 3l1 and/or Afg 3l2 or (a) variant(s) thereof are contacted in the absence of said compound to be screened for under conditions allowing OPA1 processing to occur.

8. The method of claim 2, wherein said compound capable of modulating the activity of an oligomeric complex comprising Afg 3l1 and/or Afg 3l2 or (a) variant(s) thereof is or comprises an agonist or antagonist of the activity of an oligomeric complex comprising Afg 3l1 and/or Afg 3l2 or (a) variant(s) thereof.

9. The method of claim 2, wherein said compound capable of modulating the activity of an oligomeric complex comprising Afg 3l1 and/or Afg 3l2 or (a) variant(s) thereof is or comprises an agonist or antagonist of the activity of an oligomeric complex comprising Afg 3l1 and/or Afg 3l2 or (a) variant(s) thereof, wherein said antagonist is a molecule selected from the group consisting of:
(a) a binding molecule that binds to/interacts with the oligomeric complex comprising Afg 3l1 and/or Afg 3l2 or (a) variant(s) thereof as defined in claim or binds to/interacts with a nucleic acid molecule encoding ((a) subunit(s) of) the oligomeric complex comprising Afg 3l1 and/or Afg 3l2 or (a) variant(s) thereof as defined in claim 2;
(b) a nucleic acid molecule capable of introducing an insertion of a heterologous sequence or a mutation into a nucleic acid molecule encoding ((a) subunit(s) of) the oligomeric complex comprising Afg 3l1 and/or Afg 3l2 or (a) variant(s) thereof as defined in claim 2 via in vivo mutagenesis;
(c) a nucleic acid molecule capable of reducing the expression of mRNA encoding ((a) subunit(s) of) the oligomeric complex comprising Afg 3l1 and/or Afg 3l2 or (a) variant(s) thereof as defined in claim 2 by cosuppression; and
(d) a low molecular weight compound or a small molecule.

10. The method of claim 9, wherein said binding molecule is selected form the group consisting of antibodies, affybodies, trinectins, anticalins, aptamers, PNA, DNA or RNA.

11. The method of claim 2, wherein said compound capable of modulating the activity of an oligomeric complex comprising Afg 3l1 and/or Afg 3l2 or (a) variant(s) thereof is or comprises an agonist or antagonist of the activity of an oligomeric complex comprising Afg 3l1 and/or Afg 3l2 or (a) variant(s) thereof, wherein said antagonist is a molecule selected from the group consisting of:
(A) a binding molecule that binds to/interacts with the oligomeric complex comprising Afg 3l1 and/or Afg 3l2 or (a) variant(s) thereof as defined in claim 2 or binds to/interacts with a nucleic acid molecule encoding ((a) subunit(s) of) the oligomeric complex comprising Afg 3l1 and/or Afg 3l2 or (a) variant(s) thereof as defined in claim 2;
(B) a nucleic acid molecule capable of introducing an insertion of a heterologous sequence or a mutation into a nucleic acid molecule encoding ((a) subunit(s) of) the oligomeric complex comprising Afg 3l1 and/or Afg 3l2 or (a) variant(s) thereof as defined in claim 2 via in vivo mutagenesis;
(C) a nucleic acid molecule capable of reducing the expression of mRNA encoding ((a) subunit(s) of) the oligomeric complex comprising Afg 3l1 and/or Afg 3l2 or (a) variant(s) thereof as defined in claim 2 by cosuppression; and
(D) a low molecular weight compound or a small molecule,
wherein said binding molecule is selected from the group consisting of:
(i) an antibody that binds to the polypeptide or the nucleic acid molecule selected from the group consisting of:
(a) a polypeptide comprising an amino acid sequence as depicted in SEQ ID NO 38, 40 or 42;
(b) a polypeptide comprising an amino acid sequence encoded by a nucleic acid molecule as depicted in SEQ ID NO 37, 39 or 41;
(c) a polypeptide comprising an amino acid sequence encoded by a nucleic acid molecule encoding an amino acid sequence as depicted in SEQ ID NO 38, 40 or 42;
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (b) to (c);
(f) a polypeptide comprising an amino acid sequence encoded by a nucleic acid molecule hybridizing under stringent conditions to the complement stand of a nucleic acid molecule as defined in any one of (b) to (c); and
(g) fragment of a polypeptide of any one of (b) to (f).
or to ((a) subunit(s) of) the oligomeric complex comprising Afg 3l1 and/or Afg 3l2 or (a) variant(s) thereof as defined in claim 2;
(ii) an antisense nucleotide sequence that hybridizes to the nucleic acid molecule as defined in (i);
(iii) a siRNA that interacts with the nucleic acid molecule as defined in (i);
(iv) an aptamer that binds to the polypeptide or the nucleic acid molecule as defined in (i) or to ((a) subunit(s) of) the oligomeric complex comprising Afg 3l1 and/or Afg 3l2 or (a) variant(s) thereof as defined in claim 2; and
(v) ribozyme that interacts with the nucleic acid molecule as defined in (i).

12. The method of claim 2, wherein said compound capable of modulating the activity of an oligomeric complex comprising Afg 3l1 and/or Afg 3l2 or (a) variant(s) thereof is or comprises an agonist or antagonist of the activity of an oligomeric complex comprising Afg 3l1 and/or Afg 3l2 or (a) variant(s) thereof, wherein said agonist is a molecule selected from the group consisting of:
(A) a polypeptide as defined in (a)-(g) or a nucleotide sequence comprising a nucleic acid molecule as defined in (a)-(g);
(a) a polypeptide comprising an amino acid sequence as depicted in SEQ ID NO 38, 40 or 42;
(b) a polypeptide comprising an amino acid sequence encoded by a nucleic acid molecule as depicted in SEQ ID NO 37, 39 or 41;
(c) a polypeptide comprising an amino acid sequence encoded by a nucleic acid molecule encoding an amino acid sequence as depicted in SEQ ID NO 38, 40 or 42;
(d) a polypeptide comprising an amino acid sequence having at least 50% sequence identity to the polypeptide of any one of (a) to (c);
(e) a polypeptide comprising an amino acid sequence encoded by a nucleic acid molecule having at least 50% sequence identity to the nucleic acid molecule as defined in any one of (b) to (c);
(f) a polypeptide comprising an amino acid sequence encoded by a nucleic acid molecule hybridizing under stringent conditions to the complement stand of a nucleic acid molecule as defined in any one of (b) to (c); and
(g) fragment of a polypeptide of any one of (b) to (f),
(B) a binding molecule as defined in any one of claims 9, 10 and 11 (a) and (d) being an agonistic binding molecule; and
(C) a low molecular weight compound or a small molecule.

13. The method of claim 2, wherein said activity of an oligomeric complex comprising Afg 3l1 and/or Afg 3l2 or (a) variant(s) thereof is a protease activity.

14. The method of claim 2, wherein said activity of an oligomeric complex comprising Afg 3l1 and/or Afg 3l2 or (a) variant(s) thereof is an m-AAA protease activity.

15. The method of claim 2, wherein said activity of an oligomeric complex comprising Afg 3l1 and/or Afg 3l2 or (a) variant(s) thereof is proteolytic cleavage of OPA1.

16. The method of claim 15, wherein said proteolytic cleavage of OPA1 leads to OPA1 processing.

17. The method of claim 2, wherein said OPA1 processing is characterized by (a decrease of) a certain amount of at least one large isoform of OPA1, (an increase of) a certain amount of at least one small isoform of OPA1 and/or (a decrease of) a certain ratio of at least one large versus at least one small isoform of OPA1 (compared to a control/standard).

18. The method of claim 2, wherein said altered OPA1 processing is characterized by an altered (decrease of a) certain amount of at least one large isoform of OPA1, an altered (increase of a) certain amount of at least one small isoform of OPA1 and/or an altered (decrease of a) certain ratio of at least one large versus at least one small isoform of OPA1 compared to a control/standard.

19. The method of claim 2, wherein said disorder or disease is selected from the group consisting of premature ageing, cardiomyopathy, a respiratory chain disorder, mtDNA depletion syndrome, myoclonus epilepsy, ragged-red fibers syndrome (MERRF), myopathy encephalopathy lactic acidosis, stroke-like episodes (MELAS) and optic atrophy.

20. The method of claim 17, whereby a large isoform of OPA1 has an apparent molecular weight of more than about 91 kD and whereby a small isoform of OPA1 has an apparent molecular weight of less than about 91 kD, said molecular weights being determined by SDS-PAGE analysis; and/or
whereby a large isoform of OPA1 has an apparent molecular weight of more than about 95 kD and whereby a small isoform of OPA1 has an apparent molecular weight of less than about 95 kD, said molecular weights being determined by mass spectrometry.

21. The method of claim 17, wherein said at least one large isoform of OPA1 comprises two isoforms (OPA1-L1 and OPA1-L2) and/or wherein said at least one small isoform of OPA1 comprises three isoforms (OPA1-S3, OPA1-S4 and OPA1-S5).

22. The method of claim 21, wherein said at least one large isoform of OPA1 comprises an isoform having an apparent molecular weight of about 97 kD (OPA1-L1) or an isoform having an apparent molecular weight of about 92 kD (OPA1-L2), said molecular weights being determined by SDS-PAGE analysis.

23. The method of claim 21, wherein said at least one small isoform of OPA1 comprises an isoform having an apparent molecular weight of about 88 kD (OPA1-S3), an isoform having an apparent molecular weight of about 84 kD (OPA1-S4) or an isoform having an apparent molecular weight of about 81 kD (OPA1-S5), said molecular weights being determined by SDS-PAGE analysis.

24. The method of claim 21, wherein said at least one large isoform of OPA1 comprises an isoform having an apparent molecular weight of about 104 kD (OPA1-L1) or an isoform having an apparent molecular weight of about 99 kD (OPA1-L2), said molecular weights being determined by mass spectrometry.

25. The method of claim 21, wherein said at least one small isoform of OPA1 comprises an isoform having an apparent molecular weight of about 92 kD (OPA1-S3), an isoform having an apparent molecular weight of about 89 kD (OPA1-S4) or an isoform having an apparent molecular weight of about 87 kD (OPA1-S5), said molecular weights being determined by mass spectrometry.

26. The method of claim 21, wherein
said OPA1-L1 has an apparent molecular weight of about 97 kD,
said OPA1-L2 has an apparent molecular weight of about 92 kD,
said OPA1-S3 has an apparent molecular weight of about 88 kD,
said OPA1-S4 has an apparent molecular weight of about 84 kD, and/or
said OPA1-S5 has an apparent molecular weight of about 81 kD,
said molecular weights being determined by SDS-PAGE analysis; or
wherein
said OPA1-L1 has an apparent molecular weight of about 104 kD,
said OPA1-L2 has an apparent molecular weight of about 99 kD,
said OPA1-S3 has an apparent molecular weight of about 92 kD,
said OPA1-S4 has an apparent molecular weight of about 89 kD, and/or
said OPA1-S5 has an apparent molecular weight of about 87 kD,
said molecular weights being determined by mass spectrometry.

27. The method of claim 20, wherein said SDS-PAGE is a 10% SDS-PAGE.

28. The method of claim 20, wherein said mass spectrometry is MALDI-MS or LC-MS/MS.

29. The method of claim 20, wherein said at least one large isoform of OPA1 is OPA1-L1 and/or OPA1-L2.

30. The method of claim 18, wherein said at least one small isoform of OPA1 is OPA1-S3, OPA1-S4 and/or OPA1-S5.

31. The method of claim 21, wherein
(a) OPA1-L1 and OPA1-L2 are characterized by comprising amino acid stretches or amino acid peptides comprising one or more of the following sequences:

| | |
|---|---|
| YLILGSAVGGGYTAK; | (SEQ ID No: 17) |
| TFDQWK; | (SEQ ID No: 18) |
| DMIPDLSEYK; | (SEQ ID No: 19) |
| WIVPDIVWEIDEYIDFEK; | (SEQ ID No: 20) |
| LAPDFDK; | (SEQ ID No: 21) |
| IVESLSLLK; | (SEQ ID No: 22) |
| ALPNSEDLVK; | (SEQ ID No: 23) |
| DFFTSGSPEETAFR; | (SEQ ID No: 24) |
| TRLLKLRYLILGS; | (SEQ ID No: 25) |
| and | |
| FWPARLATRLLKLRYLILGS | (SEQ ID NO: 35) |

or derivatives thereof;
(b) OPA1-S3 is characterized by comprising amino acid stretches or amino acid peptides comprising one or more of the following sequences:

| | |
|---|---|
| IVESLSLLK; | (SEQ ID No: 22) |
| DFFTSGSPEETAFR; | (SEQ ID No: 24) |
| GLLGELILLQQQIQEHEEEAR; | (SEQ ID No: 26) |
| AAGQYSTSYAQQK; | (SEQ ID No: 27) |
| and | |
| IDQLQEELLHTQLK | (SEQ ID No: 28) |

or derivatives thereof;
(c) OPA1-S4 is characterized by comprising amino acid stretches or amino acid peptides comprising one or more of the following sequences:

| | |
|---|---|
| GLLGELILLQQQIQEHEEEAR; | (SEQ ID No: 26) |
| AAGQYSTSYAQQK; | (SEQ ID No: 27) |
| and | |
| IDQLQEELLHTQLK | (SEQ ID No: 28) |

or derivatives thereof; and/or
(d) OPA1-S5 is characterized by comprising amino acid stretches or amino acid peptides comprising the following sequence:

| | |
|---|---|
| IDQLQEELLHTQLK | (SEQ ID No: 28) |

or derivatives thereof.

32. The method of claim 21, wherein
(a) OPA1-L2 is characterized by not comprising amino acid stretches or amino acid peptides comprising one or more of the following sequences:

| | |
|---|---|
| GLLGELILLQQQIQEHEEEAR; | (SEQ ID No: 26) |
| and | |
| AAGQYSTSYAQQK; | (SEQ ID No: 27) |

or derivatives thereof; and/or
(b) OPA1-S3 is characterized by not comprising amino acid stretches or amino acid peptides comprising one or more of the following sequences:

| | |
|---|---|
| YLILGSAVGGGYTAK; | (SEQ ID No: 17) |
| TFDQWK; | (SEQ ID No: 18) |
| DMIPDLSEYK; | (SEQ ID No: 19) |
| WIVPDIVWEIDEYIDFEK; | (SEQ ID No: 20) |
| LAPDFDK; | (SEQ ID No: 21) |
| ALPNSEDLVK; | (SEQ ID No: 23) |
| FWPARLATRLLKLRYLILGS; | (SEQ ID NO: 35) |
| and | |
| TRLLKLRYLILGS | (SEQ ID No: 25) |

or derivatives thereof;
(c) OPA1-S4 is characterized by not comprising amino acid stretches or amino acid peptides comprising one or more of the following sequences:

| | |
|---|---|
| YLILGSAVGGGYTAK; | (SEQ ID No: 17) |
| TFDQWK; | (SEQ ID No: 18) |
| DMIPDLSEYK; | (SEQ ID No: 19) |
| WIVPDIVWEIDEYIDFEK; | (SEQ ID No: 20) |
| LAPDFDK; | (SEQ ID No: 21) |
| IVESLSLLK; | (SEQ ID No: 22) |
| ALPNSEDLVK; | (SEQ ID No: 23) |
| DFFTSGSPEETAFR; | (SEQ ID No: 24) |
| FWPARLATRLLKLRYLILGS; | (SEQ ID NO: 35) |
| and | |
| TRLLKLRYLILGS | (SEQ ID No: 25) |

or derivatives thereof; and/or
(d) OPA1-S5 is characterized by not comprising amino acid stretches or amino acid peptides comprising one or more of the following sequences:

| | |
|---|---|
| YLILGSAVGGGYTAK; | (SEQ ID No: 17) |
| TFDQWK; | (SEQ ID No: 18) |
| DMIPDLSEYK; | (SEQ ID No: 19) |
| WIVPDIVWEIDEYIDFEK; | (SEQ ID No: 20) |
| LAPDFDK; | (SEQ ID No: 21) |
| IVESLSLLK; | (SEQ ID No: 22) |
| ALPNSEDLVK; | (SEQ ID No: 23) |
| DFFTSGSPEETAFR; | (SEQ ID No: 24) |
| TRLLKLRYLILGS; | (SEQ ID No: 25) |
| GLLGELILLQQQIQEHEEEAR; | (SEQ ID No: 26) |
| and | |
| AAGQYSTSYAQQK; | (SEQ ID No: 27) |

or derivatives thereof.

33. A compound capable of modulating the activity of an oligomeric complex comprising Afg 3l1 and/or Afg 3l2 or (a) variant(s) thereof for the treatment, prevention and/or amelioration of
(i) a disorder or disease correlated with mitochondrial dysfunction or a mitochondrial disorder or disease; or
(ii) a disorder or disease characterized by an altered OPA1 processing,
wherein said oligomeric complex is defined as in claim 2, said compound is defined as in claim 2, said disorder or disease is defined as in claim 2 and/or said OPA1 processing is defined as in claim 2.


SEQUENCE LISTING (42 sequences)

SEQ ID NO: 1 - DNA, 3048 bp, Homo sapiens
  CDS (1)..(3048)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctaattcagaagaccttgtaaagttagcaccagactttgacaagattgttgaaagccttagcttattgaaggacttttttacctcaggtcacaaattggttagtgaagtcataggagcttctgacctacttctcttgttaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 2 - protein, 1015 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPNSEDLVKLAPDFDKIVESLSLLKDFFTSGHKLVSEVIGASDLLLLLGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 3 - DNA, 2994 bp, Homo sapiens
  CDS (1)..(2994)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctaattcagaagaccttgtaaagttagcaccagactttgacaagattgttgaaagccttagcttattgaaggacttttttacctcaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 4 - protein, 997 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPNSEDLVKLAPDFDKIVESLSLLKDFFTSGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 5 - DNA, 2940 bp, Homo sapiens
  CDS (1)..(2940)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggtcacaaattggttagtgaagtcataggagcttctgacctacttctcttgttaggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 6 - protein, 979 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGHKLVSEVIGASDLLLLLGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 7 - DNA, 2937 bp, Homo sapiens
  CDS (1)..(2937)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctaattcagaagaccttgtaaagttagcaccagactttgacaagattgttgaaagccttagcttattgaaggacttttttacctcaggtcacaaattggttagtgaagtcataggagcttctgacctacttctcttgttaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 8 - protein, 978 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPNSEDLVKLAPDFDKIVESLSLLKDFFTSGHKLVSEVIGASDLLLLLGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 9 - DNA, 2886 bp, Homo sapiens
  CDS (1)..(2886)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggttctccggaagaaacggcgtttagagcaacagatcgtggatctgaaagtgacaagcattttagaaagggtctgcttggtgagctcattctcttacaacaacaaattcaagagcatgaagaggaagcgcgcagagccgctggccaatatagcacgagctatgcccaacagaagcgcaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 10 - protein, 961 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGSPEETAFRATDRGSESDKHFRKGLLGELILLQQQIQEHEEEARRAAGQYSTSYAQQKRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 11 - DNA, 2829 bp, Homo sapiens
  CDS (1)..(2829)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggtcacaaattggttagtgaagtcataggagcttctgacctacttctcttgttaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 12 - protein, 942 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGHKLVSEVIGASDLLLLLGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 13 - DNA, 2775 bp, Homo sapiens
  CDS (1)..(2775)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 14 - protein, 924 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 15 - DNA, 2883 bp, Homo sapiens
  CDS (1)..(2883)
atgtggcgactacgtcgggccgctgtggcctgtgaggtctgccagtctttagtgaaacacagctctggaataaaaggaagtttaccactacaaaaactacatctggtttcacgaagcatttatcattcacatcatcctaccttaaagcttcaacgaccccaattaaggacatcctttcagcagttctcttctctgacaaaccttcctttacgtaaactgaaattctctccaattaaatatggctaccagcctcgcaggaatttttggccagcaagattagctacgagactcttaaaacttcgctatctcatactaggatcggctgttgggggtggctacacagccaaaaagacttttgatcagtggaaagatatgataccggaccttagtgaatataaatggattgtgcctgacattgtgtgggaaattgatgagtatatcgattttgagaaaattagaaaagcccttcctaattcagaagaccttgtaaagttagcaccagactttgacaagattgttgaaagccttagcttattgaaggacttttttacctcaggttctccggaagaaacggcgtttagagcaacagatcgtggatctgaaagtgacaagcattttagaaaggtgtcagacaaagagaaaattgaccaacttcaggaagaacttctgcacactcagttgaagtatcagagaatcttggaacgattagaaaaggagaacaaagaattgagaaaattagtattgcagaaagatgacaaaggcattcatcatagaaagcttaagaaatctttgattgacatgtattctgaagttcttgatgttctctctgattatgatgccagttataatacgcaagatcatctgccacgggttgttgtggttggagatcagagtgctggaaagactagtgtgttggaaatgattgcccaagctcgaatattcccaagaggatctggggagatgatgacacgttctccagttaaggtgactctgagtgaaggtcctcaccatgtggccctatttaaagatagttctcgggagtttgatcttaccaaagaagaagatcttgcagcattaagacatgaaatagaacttcgaatgaggaaaaatgtgaaagaaggctgtaccgttagccctgagaccatatccttaaatgtaaaaggccctggactacagaggatggtgcttgttgacttaccaggtgtgattaatactgtgacatcaggcatggctcctgacacaaaggaaactattttcagtatcagcaaagcttacatgcagaatcctaatgccatcatactgtgtattcaagatggatctgtggatgctgaacgcagtattgttacagacttggtcagtcaaatggaccctcatggaaggagaaccatattcgttttgaccaaagtagacctggcagagaaaaatgtagccagtccaagcaggattcagcagataattgaaggaaagctcttcccaatgaaagctttaggttattttgctgttgtaacaggaaaagggaacagctctgaaagcattgaagctataagagaatatgaagaagagttttttcagaattcaaagctcctaaagacaagcatgctaaaggcacaccaagtgactacaagaaatttaagccttgcagtatcagactgcttttggaaaatggtacgagagtctgttgaacaacaggctgatagtttcaaagcaacacgttttaaccttgaaactgaatggaagaataactatcctcgcctgcgggaacttgaccggaatgaactatttgaaaaagctaaaaatgaaatccttgatgaagttatcagtctgagccaggttacaccaaaacattgggaggaaatccttcaacaatctttgtgggaaagagtatcaactcatgtgattgaaaacatctaccttccagctgcgcagaccatgaattcaggaacttttaacaccacagtggatatcaagcttaaacagtggactgataaacaacttcctaataaagcagtagaggttgcttgggagaccctacaagaagaattttcccgctttatgacagaaccgaaagggaaagagcatgatgacatatttgataaacttaaagaggccgttaaggaagaaagtattaaacgacacaagtggaatgactttgcggaggacagcttgagggttattcaacacaatgctttggaagaccgatccatatctgataaacagcaatgggatgcagctatttattttatggaagaggctctgcaggctcgtctcaaggatactgaaaatgcaattgaaaacatggtgggtccagactggaaaaagaggtggttatactggaagaatcggacccaagaacagtgtgttcacaatgaaaccaagaatgaattggagaagatgttgaaatgtaatgaggagcacccagcttatcttgcaagtgatgaaataaccacagtccggaagaaccttgaatcccgaggagtagaagtagatccaagcttgattaaggatacttggcatcaagtttatagaagacattttttaaaaacagctctaaaccattgtaacctttgtcgaagaggtttttattactaccaaaggcattttgtagattctgagttggaatgcaatgatgtggtcttgttttggcgtatacagcgcatgcttgctatcaccgcaaatactttaaggcaacaacttacaaatactgaagttaggcgattagagaaaaatgttaaagaggtattggaagattttgctgaagatggtgagaagaagattaaattgcttactggtaaacgcgttcaactggcggaagacctcaagaaagttagagaaattcaagaaaaacttgatgctttcattgaagctcttcatcaggagaaataa

SEQ ID NO: 16 - protein, 960 aa, Homo sapiens
MWRLRRAAVACEVCQSLVKHSSGIKGSLPLQKLHLVSRSIYHSHHPTLKLQRPQLRTSFQQFSSLTNLPLRKLKFSPIKYGYQPRRNFWPARLATRLLKLRYLILGSAVGGGYTAKKTFDQWKDMIPDLSEYKWIVPDIVWEIDEYIDFEKIRKALPNSEDLVKLAPDFDKIVESLSLLKDFFTSGSPEETAFRATDRGSESDKHFRKVSDKEKIDQLQEELLHTQLKYQRILERLEKENKELRKLVLQKDDKGIHHRKLKKSLIDMYSEVLDVLSDYDASYNTQDHLPRVVVVGDQSAGKTSVLEMIAQARIFPRGSGEMMTRSPVKVTLSEGPHHVALFKDSSREFDLTKEEDLAALRHEIELRMRKNVKEGCTVSPETISLNVKGPGLQRMVLVDLPGVINTVTSGMAPDTKETIFSISKAYMQNPNAIILCIQDGSVDAERSIVTDLVSQMDPHGRRTIFVLTKVDLAEKNVASPSRIQQIIEGKLFPMKALGYFAVVTGKGNSSESIEAIREYEEEFFQNSKLLKTSMLKAHQVTTRNLSLAVSDCFWKMVRESVEQQADSFKATRFNLETEWKNNYPRLRELDRNELFEKAKNEILDEVISLSQVTPKHWEEILQQSLWERVSTHVIENIYLPAAQTMNSGTFNTTVDIKLKQWTDKQLPNKAVEVAWETLQEEFSRFMTEPKGKEHDDIFDKLKEAVKEESIKRHKWNDFAEDSLRVIQHNALEDRSISDKQQWDAAIYFMEEALQARLKDTENAIENMVGPDWKKRWLYWKNRTQEQCVHNETKNELEKMLKCNEEHPAYLASDEITTVRKNLESRGVEVDPSLIKDTWHQVYRRHFLKTALNHCNLCRRGFYYYQRHFVDSELECNDVVLFWRIQRMLAITANTLRQQLTNTEVRRLEKNVKEVLEDFAEDGEKKIKLLTGKRVQLAEDLKKVREIQEKLDAFIEALHQEK

SEQ ID NO: 17 - protein, 15 aa, artificial sequence
  /note=“Description of artificial sequence OPA1”
YLILGSAVGGGYTAK

SEQ ID NO: 18 - protein, 6 aa, artificial sequence
  /note=“Description of artificial sequence fragment of OPA1”
TFDQWK

SEQ ID NO: 19 - protein, 10 aa, artificial sequence
  /note=“Description of artificial sequence fragment of OPA1”
DMIPDLSEYK

SEQ ID NO: 20 - protein, 18 aa, artificial sequence
  /note=“Description of artificial sequence fragment of OPA1”
WIVPDIVWEIDEYIDFEK

SEQ ID NO: 21 - protein, 7 aa, artificial sequence
  /note=“Description of artificial sequence fragment of OPA1”
LAPDFDK

SEQ ID NO: 22 - protein, 9 aa, artificial sequence
  /note=“Description of artificial sequence fragment of OPA1”
IVESLSLLK

SEQ ID NO: 23 - protein, 10 aa, artificial sequence
  /note=“Description of artificial sequence fragment of OPA1”
ALPNSEDLVK

SEQ ID NO: 24 - protein, 14 aa, artificial sequence
  /note=“Description of artificial sequence fragment of OPA1”
DFFTSGSPEETAFR

SEQ ID NO: 25 - protein, 13 aa, artificial sequence
  /note=“Description of artificial sequence fragment of OPA1”
TRLLKLRYLILGS

SEQ ID NO: 26 - protein, 21 aa, artificial sequence
  /note=“Description of artificial sequence fragment of OPA1”
GLLGELILLQQQIQEHEEEAR

SEQ ID NO: 27 - protein, 13 aa, artificial sequence
  /note=“Description of artificial sequence fragment of OPA1”
AAGQYSTSYAQQK

SEQ ID NO: 28 - protein, 14 aa, artificial sequence
  /note=“Description of artificial sequence fragment of OPA1”
IDQLQEELLHTQLK

SEQ ID NO: 29 - protein, 24 aa, artificial sequence
  /note=“Description of artificial sequence fragment of OPA1”
CDLKKVREIQEKLDAFIEALHQEK

SEQ ID NO: 30 - protein, 23 aa, artificial sequence
  /note=“Description of artificial sequence fragment of OPA1”
DLKKVREIQEKLDAFIEALHQEK

SEQ ID NO: 31 - protein, 37 aa, artificial sequence
  /note=“Description of artificial sequence fragment of OPA1”
EYKWIVPDIVWEIDEYIDFGHKLVSEVIGASDLLLLL

SEQ ID NO: 32 - protein, 42 aa, artificial sequence
  /note=“Description of artificial sequence fragment of OPA1”
EYKWIVPDIVWEIDEYIDFGSPEETAFRATDRGSESDKHFRK

SEQ ID NO: 33 - protein, 59 aa, artificial sequence
  /note=“Description of artificial sequence fragment of OPA1”
EKIRKALPNSEDLVKLAPDFDKIVESLSLLKDFFTSGSPEETAFRATDRGSESDKHFRK

SEQ ID NO: 34 - protein, 60 aa, artificial sequence
  /note=“Description of artificial sequence fragment of OPA1”
GSPEETAFRATDRGSESDKHFRKVSDKEKIDQLQEELLHTQLKYQRILERLEKENKELRK

SEQ ID NO: 35 - protein, 20 aa, artificial sequence
  /note=“Description of artificial sequence fragment of OPA1”
FWPARLATRLLKLRYLILGS

SEQ ID NO: 36 - protein, 16 aa, artificial sequence
  /note=“Description of artificial sequence fragment of OPA1”
CEKQRQRRLQAAGRWF

SEQ ID NO: 37 - DNA, 2370 bp, mus musculus
  CDS (1)..(2370)
atgttactgcggctggtgggggcggcgggcagtcgagccctggcctggcctttctccaagctgtggcgatgtggcggatgcgcagggagcggcgggacggtctggagcagcgtgagggcctgtggcattgctctgcagggtcatctggggagatgctcgcagcagctggctctgcagggaaaactgacttcattttccccgaggctgtattcaaaacctcccagagggtttgagaagttttttaagaataagaagaacagaaaaagtgcaagcccaggaaattcagtacctccaaaaaaagaaccaaaaaatgctggccctggaggagatggaggcaacagaggagggaaaggagatgattttccctggtggaaacggatgcaaaagggagaatttccttgggacgacaaggacttccggagcctggctgttttgggggctggtgtggctgcgggatttttatatttttatttccgagatcccggaaaagagatcacctggaaacacttcgtgcagtattacctggccagaggtctggtggaccggctagaggttgtgaacaagcagtttgtgcgtgttattcctgttcctgggacgacatctgagaggttcgtgtggtttaacattggcagtgttgacacctttgaacggaacctcgagtctgctcagtgggagctgggcattgagcccaccaaccaggctgcggtggtctacactactgagagtgatggctcttttcttagaagtcttgtgcccactctggtcctggttagcatcctcctatatgctatgaggaggggtccaatggggactggtcgcggtgggcgaggaggaggcctcttcagtgttggtgagacaacagccaagatcttaaagaacaacatcgatgtgcggtttgcagatgtggctggctgtgaagaagccaaactggaaattatggagtttgtgaatttcctgaagaacccaaagcaatatcaggacttaggagccaaaattccaaagggagcgatgctcactggtccacctggtactggcaaaacacttcttgcaaaagcaactgctggggaggccaacgtgcccttcatcaccgtgaatgggtcggaattcctggaaatgtttgttggtgttgggccagcacgggtccgtgacatgtttgcaatggcccgaaaacacgctccttgtattttattcattgatgagattgatgcaattggcagaaagcgaggccgtggccacctgggaggccagagtgagcaggaaaacactttaaaccagatgcttgtggagatggacgggttcaactcttccactaatgtggtagtgttagccggcaccaatcgccctgatatccttgacccagccctgacacggcctggccggtttgaccgtcagatctacatcggtccccctgatatcaaaggcaggtcctctattttcaaggtccacttgcgtccactcaagctggacggaagcctcagcaaggacgctctttcgaggaagctggcagctcttactccaggcttcactggtgctgatatttccaatgtttgcaatgaagcagcactgattgctgcccgccacctcagcccttctgtccaggagcggcactttgagcaagccatcgagagggtcattggaggccttgagaagaagacccaggtcctacaacccagtgaaaagacaactgtagcctaccacgaggctgggcatgcagtggtgggctggttcttggagcatgcagaccctctgctgaaggtgtccatcatacctcgaggcaaggggcttggctacgcccagtaccttccccgcgagcagttcctctacacacgagagcagctcttcgaccgcatgtgtatgatgctggggggtagggtagctgagcagctgttctttggtcagatcaccaccggagctcaggacgacctgaggaaggtcacccagagtgcctatgcccagattgtgcagtttgggatgagtgagaagctgggccaggtgtcctttgacttccccagacaaggcgaaaccatggtggagaagccatacagtgaggctactgcccagctcattgatgaagaggtccggtgcctcgtcaggtctgcctataatcggaccctggagctgctcacacagtgccgggagcaggtggagaaggttggcaggcgtctcctggagaaagaagtgctggagaaagccgacatgatagagctcttgggccctcggccctttgcagagaagtccacctatgaggaatttgtagagggcaccggcagcctagaggaggacacatcccttcctgaggggctgaaagactggaataaggggcgggaggaaggaggcactgagcggggcttgcaggagagccctgtgtag

SEQ ID NO: 38 - protein, 789 aa, mus musculus
MLLRLVGAAGSRALAWPFSKLWRCGGCAGSGGTVWSSVRACGIALQGHLGRCSQQLALQGKLTSFSPRLYSKPPRGFEKFFKNKKNRKSASPGNSVPPKKEPKNAGPGGDGGNRGGKGDDFPWWKRMQKGEFPWDDKDFRSLAVLGAGVAAGFLYFYFRDPGKEITWKHFVQYYLARGLVDRLEVVNKQFVRVIPVPGTTSERFVWFNIGSVDTFERNLESAQWELGIEPTNQAAVVYTTESDGSFLRSLVPTLVLVSILLYAMRRGPMGTGRGGRGGGLFSVGETTAKILKNNIDVRFADVAGCEEAKLEIMEFVNFLKNPKQYQDLGAKIPKGAMLTGPPGTGKTLLAKATAGEANVPFITVNGSEFLEMFVGVGPARVRDMFAMARKHAPCILFIDEIDAIGRKRGRGHLGGQSEQENTLNQMLVEMDGFNSSTNVVVLAGTNRPDILDPALTRPGRFDRQIYIGPPDIKGRSSIFKVHLRPLKLDGSLSKDALSRKLAALTPGFTGADISNVCNEAALIAARHLSPSVQERHFEQAIERVIGGLEKKTQVLQPSEKTTVAYHEAGHAVVGWFLEHADPLLKVSIIPRGKGLGYAQYLPREQFLYTREQLFDRMCMMLGGRVAEQLFFGQITTGAQDDLRKVTQSAYAQIVQFGMSEKLGQVSFDFPRQGETMVEKPYSEATAQLIDEEVRCLVRSAYNRTLELLTQCREQVEKVGRRLLEKEVLEKADMIELLGPRPFAEKSTYEEFVEGTGSLEEDTSLPEGLKDWNKGREEGGTERGLQESPV

SEQ ID NO: 39 - DNA, 2394 bp, homo sapiens
  CDS (1)..(2394)
atggcgcaccgctgtttgcggctgtggggccggggcggctgctggccccgcggcctacagcagctcctcgtgcctggcggcgtgggcccgggcgagcagccctgcctccggacgctttaccgatttgttacaactcaagcaagggccagcagaaattctcttttgacagatataattgctgcttatcaaagattctgttctcgacccccaaaaggatttggaaaatactttcctaatggaaaaaatggaaaaaaagctagtgaacctaaagaagttatgggagagaaaaaagaatcaaagccagctgctaccacacgctcttctggaggaggaggtggtggcggtggaaaacgaggtggcaagaaagatgattctcactggtggtccaggtttcagaagggtgacattccatgggacgacaaggatttcaggatgttcttcctctggactgctctgttctggggtggagtcatgttttacttgctgctcaagagatccgggagagaaatcacttggaaggactttgtcaataactatctttcaaaaggagtagtagacagattggaagtcgtcaacaagcgttttgttcgagtgacctttacaccaggaaaaactcctgttgatgggcaatacgtttggtttaatattggcagtgtggacacctttgaacggaatctggaaactttacagcaggaattgggcatagaaggagaaaatcgggtgcctgttgtctacattgctgaaagtgatggctcttttctgctgagcatgctgcctacggtgctcatcatcgccttcttgctctacaccatcagaagagggcctgctggcattggccggacaggccgagggatgggcggactcttcagtgtcggagaaaccactgccaaggtcttaaaggatgaaattgatgtgaagttcaaagatgtggctggctgtgaggaggccaagctagagatcatggaatttgtgaatttcttgaaaaacccaaagcagtatcaagacctaggagcaaaaatcccaaagggtgccattctcactggtcctccaggcactgggaagacgctgctagctaaggccacagccggagaagccaatgtccccttcatcaccgttagtggatctgagtttttggagatgttcgttggtgtgggccctgctagagtccgagacttatttgcccttgctcggaagaatgccccttgcatcctcttcatcgatgaaatcgatgcggtgggaaggaagagaggaagaggcaactttggagggcagagtgagcaggagaacacactcaaccagctgctggtggagatggatggttttaatacaacaacaaatgtcgtcattttggccggcaccaatcgaccagatatcctggaccccgcgctacttaggccggggcgtttcgacaggcagatctttattggaccaccagacataaaaggaagagcttctattttcaaagttcatctccgaccgctaaaactggacagtaccctggagaaggataaattggcaagaaaactggcatctttaactccagggttttcaggtgctgatgttgctaatgtctgtaatgaagctgcgttgattgctgcaaggcatctgtcagattccataaatcagaaacactttgaacaggcaattgagcgagtgattggtggcttagagaagaaaacgcaggttctgcagcctgaggagaagaagactgtggcataccacgaagcaggccatgcggttgccggctggtatctggagcacgcagacccgcttttaaaggtatccatcatcccacgtggcaaaggactaggttatgctcagtatttaccaaaagaacaatacctctataccaaagagcagctcttggataggatgtgtatgactttaggtggtcgagcctctgaagaaatcttctttggaagaattacaactggtgctcaagatgacttgagaaaagtaactcagagtgcatatgcccaaattgttcagtttggcatgaatgaaaaggttgggcaaatctcctttgacctcccacgtcagggggacatggtattggagaaaccttacagtgaagccactgcaagattgatagatgatgaagtacgaatacttattaatgatgcttataaaagaacagtagctcttctcacagaaaagaaagctgacgtggagaaggttgctcttctgttgttagaaaaagaagtattagataagaatgatatggttgaacttttgggccccagaccatttgcggaaaaatctacctatgaagaatttgtggaaggcactggcagcttggatgaggacacctcacttccagaaggccttaaggactggaacaaggagcgggaaaaggagaaagaggagcccccgggtgagaaagttgccaactag

SEQ ID NO: 40 - protein, 797 aa, homo sapiens
MAHRCLRLWGRGGCWPRGLQQLLVPGGVGPGEQPCLRTLYRFVTTQARASRNSLLTDIIAAYQRFCSRPPKGFGKYFPNGKNGKKASEPKEVMGEKKESKPAATTRSSGGGGGGGGKRGGKKDDSHWWSRFQKGDIPWDDKDFRMFFLWTALFWGGVMFYLLLKRSGREITWKDFVNNYLSKGVVDRLEVVNKRFVRVTFTPGKTPVDGQYVWFNIGSVDTFERNLETLQQELGIEGENRVPVVYIAESDGSFLLSMLPTVLIIAFLLYTIRRGPAGIGRTGRGMGGLFSVGETTAKVLKDEIDVKFKDVAGCEEAKLEIMEFVNFLKNPKQYQDLGAKIPKGAILTGPPGTGKTLLAKATAGEANVPFITVSGSEFLEMFVGVGPARVRDLFALARKNAPCILFIDEIDAVGRKRGRGNFGGQSEQENTLNQLLVEMDGFNTTTNVVILAGTNRPDILDPALLRPGRFDRQIFIGPPDIKGRASIFKVHLRPLKLDSTLEKDKLARKLASLTPGFSGADVANVCNEAALIAARHLSDSINQKHFEQAIERVIGGLEKKTQVLQPEEKKTVAYHEAGHAVAGWYLEHADPLLKVSIIPRGKGLGYAQYLPKEQYLYTKEQLLDRMCMTLGGRASEEIFFGRITTGAQDDLRKVTQSAYAQIVQFGMNEKVGQISFDLPRQGDMVLEKPYSEATARLIDDEVRILINDAYKRTVALLTEKKADVEKVALLLLEKEVLDKNDMVELLGPRPFAEKSTYEEFVEGTGSLDEDTSLPEGLKDWNKEREKEKEEPPGEKVAN

SEQ ID NO: 41 - DNA, 2409 bp, mus musculus
  CDS (1)..(2409)
atggcccaccgctgcctgctgctgtggagccggggcggctgccgtcgcggccttcctcccctgctcgtgcccagaggttgcctgggtccggaccggcggccctgcctccgtacgctctatcaatatgctactgtccagacagcaagcagcaggcgttctctgctgagggatgtaattgctgcttatcaaagattctgttctcgacctcccaaaggatttgaaaagtactttcctaatgggaaaaacggaaaaaaggccagtgagcctaaggaggctgttggagaaaaaaaagaaccacagccctcgggcccccagccttctggaggtgcaggtggtgggggagggaagcgccgtggcaagaaagaagattctcactggtggtccaggttccagaagggtgacttcccatgggatgacaaggatttcaggatgtactttctctggactgctcttttttggggtggagtcatgatttacttcgtgttcaagagctctgggagagaaatcacgtggaaagactttgtcaataactatctttctaagggcgtggtggacagactagaagttgtcaacaagcgttttgttcgtgtgacctttacaccaggaaaaactccggttgatgggcaatacgtctggtttaatattggcagtgttgacacatttgagcggaatctggagactttgcagcaagaattgggcatagaaggggagaaccgggtccctgtggtttatattgctgagagcgatggctccttcctgctgagcatgttgcccaccgtactcattatcgcttttctactctacaccataagaagagggcccgctggcattggtcggaccggccggggaatgggtggactcttcagcgttggggaaaccacagccaaggtcttaaaggatgagatagatgtgaagtttaaagatgtggctggctgtgaggaggccaagctagaaataatggaattcgtgaatttcttgaaaaacccaaagcaatatcaagacctaggagcaaaaatcccaaagggtgccattctcaccggtcccccaggtactgggaagacgctgctagctaaggccacagctggagaagccaatgtcccctttatcactgtgagcggatctgagtttctggagatgtttgttggcgttggtccagccagagtccgagacttatttgcccttgctcggaagaatgcgccttgcattctcttcattgatgagattgatgctgtgggaaggaagcgcggcagaggcaacttcggtgggcagagcgagcaggagaacacactcaaccagctgcttgtggagatggacggcttcaacacaaccaccaatgtggtcatcctggcaggcacaaatcgaccagacatcctggatccagctctgttgagaccaggccgctttgacaggcagatttttattggacccccagacataaaaggacgagcctcaatcttcaaagttcaccttcgaccattgaagctggacagtgccttggaaaaagataaattggccagaaaactggcgtccttaactccagggttttcaggcgctgatgttgccaatgtctgcaatgaagctgctttgattgctgcaagacacctttcagatgccattaatgagaagcacttcgaacaagcgattgagcgagtgattggaggcttggagaaaaaaacccaagttctgcagcctgaggagaagaagacggtggcttaccacgaagcaggccatgcggtcgctggctggtatctggagcatgcagacccactcttaaaggtctccatcatcccgcgtggcaaggggctgggctatgctcagtacttgcccaaggagcagtatctgtacacaaaggagcagctgctggacaggatgtgcatgactctgggcggccgtgtctccgaggagatcttctttgggagaattacaaccggtgcccaggacgacttgaggaaggttacccagagtgcctatgcccagatcgttcagtttggcatgaacgagaaagtggggcagatctcctttgacctcccacgacagggggacatggtgttagagaagccttacagtgaggccactgcgaggatgatagacgatgaagtgaggatactcatcagcgatgcctacagaaggacggtggctcttctcacagagaagaaggctgacgtggagaaggtcgctctcttactgttagaaaaggaagtcctagacaagaatgacatggtccagcttctcggtcccagaccatttacagaaaagtccacatatgaagaatttgtggaaggcactggcagcttagacgaggacacttctcttcctgaaggccttcaggattggaacaaggagcgggagaaggaggagaagaaggagaaggagaaggaggagccgctgaatgagaaggttgtcagctag

SEQ ID NO: 42 - protein, 802 aa, mus musculus
MAHRCLLLWSRGGCRRGLPPLLVPRGCLGPDRRPCLRTLYQYATVQTASSRRSLLRDVIAAYQRFCSRPPKGFEKYFPNGKNGKKASEPKEAVGEKKEPQPSGPQPSGGAGGGGGKRRGKKEDSHWWSRFQKGDFPWDDKDFRMYFLWTALFWGGVMIYFVFKSSGREITWKDFVNNYLSKGVVDRLEVVNKRFVRVTFTPGKTPVDGQYVWFNIGSVDTFERNLETLQQELGIEGENRVPVVYIAESDGSFLLSMLPTVLIIAFLLYTIRRGPAGIGRTGRGMGGLFSVGETTAKVLKDEIDVKFKDVAGCEEAKLEIMEFVNFLKNPKQYQDLGAKIPKGAILTGPPGTGKTLLAKATAGEANVPFITVSGSEFLEMFVGVGPARVRDLFALARKNAPCILFIDEIDAVGRKRGRGNFGGQSEQENTLNQLLVEMDGFNTTTNVVILAGTNRPDILDPALLRPGRFDRQIFIGPPDIKGRASIFKVHLRPLKLDSALEKDKLARKLASLTPGFSGADVANVCNEAALIAARHLSDAINEKHFEQAIERVIGGLEKKTQVLQPEEKKTVAYHEAGHAVAGWYLEHADPLLKVSIIPRGKGLGYAQYLPKEQYLYTKEQLLDRMCMTLGGRVSEEIFFGRITTGAQDDLRKVTQSAYAQIVQFGMNEKVGQISFDLPRQGDMVLEKPYSEATARMIDDEVRILISDAYRRTVALLTEKKADVEKVALLLLEKEVLDKNDMVQLLGPRPFTEKSTYEEFVEGTGSLDEDTSLPEGLQDWNKEREKEEKKEKEKEEPLNEKVVS